(19) United States
(12) Patent Application Publication
(10) Pub. No.: US 2011/0033887 A1
(43) Pub. Date: Feb. 10, 2011

(54) Three-Dimensional Microfabricated Bioreactors with Embedded Capillary Network

(76) Inventors: Nicholas X. Fang, Champaign, IL (US); Chunguang Xia, Urbana, IL (US); Andrew Cox, Batavia, IL (US)

Correspondence Address: GREENLEE SULLIVAN P.C., 4875 PEARL EAST CIRCLE, SUITE 200, BOULDER, CO, 80301, US

(21) Appl. No.: 12/679,497

(22) PCT Filed: Sep. 24, 2008
(86) PCT No.: PCT/US08/77503

§ 371 Date: Oct. 25, 2010

Related U.S. Application Data
(60) Provisional application No. 60/974,699, filed on Sep. 24, 2007.

(51) Int. Cl. C12M 3/00; G03F 7/20; C12P 1/00; G01D 18/00

(52) U.S. Cl. 435/41; 430/320; 435/303.1; 73/1.01

(57) ABSTRACT

In an aspect, the present invention uses projection micro stereolithography to generate three-dimensional microvessel networks that are capable of supporting and fostering growth of a cell population. For example, provided is a method of making a microvascularized bioreactor via layer-by-layer polymerization of a photocurable liquid composition with repeated patterns of illumination, wherein each layer corresponds to a layer of the desired microvessel network. The plurality of layers are assembled to make a microvascular network. Support structures having different etch rates than the structures that make up the network provides access to manufacturing arbitrary geometries that cannot be made by conventional methods. A cell population is introduced to the external wall of the network to obtain a microvascularized bioreactor. Provided are various methods and related bioreactors, wherein the network wall has a permeability to a biological material that varies within and along the network.

### CROSS-REFERENCE TO RELATED APPLICATIONS

This application claims the benefit of priority to U.S. Provisional Patent Application 60/974,699 filed Sep. 24, 2007 which is hereby incorporated by reference in its entirety to the extent not inconsistent with the disclosure herein.

### BACKGROUND OF THE INVENTION

There is a great deal of interest in systems that provide the capacity for expansion of a cell population for a number of reasons. First, there is increasing demand for cell secreted products, including mammalian cell secreted products, that can be of benefit for human use. The products include monoclonal antibodies, vaccines, hormones, growth factors, enzymes and other recombinant DNA products. For any of these products to be of commercial use, the product must be economically produced. Accordingly, systems capable of culturing relatively dense three-dimensional cell cultures to provide higher concentration of the product are desirable. Similarly, there is increasing interest in biological systems that generate a secreted product useful in nonbiological applications, such as systems capable of making a biofuel. Second, in the field of tissue engineering, there is a need for tissue implants having three-dimensional shape for insertion into patients having a tissue defect. A beneficial method of implantation involves harvesting a relatively small number of cells from the patient and expanding those cells in a system that provides for growth in a three-dimensional volume. After sufficient in vitro expansion, the three-dimensional in vitro system may be implanted into the patient. Immune response is minimized since the patient's own cells are used. A common requirement for all these applications are a system having a three-dimensional microvascularized network of vessels capable of sustaining and fostering relatively high volume density cell growth.

Reconstructive surgery is one area of particular use for three-dimensional microvascularized culture systems. Reconstructive surgery is performed to recover function and appearance of the damaged tissues, especially following major cancer resections and trauma. It is estimated that more than one million reconstructive surgery procedures are performed every year. And the reconstructive surgery has changed from “climbing ladder” to “riding elevator” (Dunn et al. Plast Rectonstr Surg 2001, 107:283), in which case flaps are preferably used in the reconstructive procedures. The free flap is the most successful procedure. A free flap is a block of tissue with an inherent microcirculatory network. This free flap is usually is removed from a different region of the patient that is relatively close to the defective site. It is based on the concept of angiosome (Taylor et al. Br J Plast Surg 1987, 113-141). However the nature of sacrificing one part of body for another limits the application of free flap in practice. To minimize attendant damage to the patient, alternative tissue sources for reconstructive surgery are desired.

Apart from the free flaps, culturing tissue in vitro using patient's cells is the most attractive way to supply tissues for reconstructive surgery, since there is no foreign body reaction. However due to the lack of microcirculatory system at the early stage of the culture, no matter in vitro or in vivo, there is only very limited success. It takes on the order of days for revascularization to occur (even with growth factors); whereas the time scale for cell death from hypoxia is on the order of hours. Therefore, without capillary perfusion the metabolism during cell growth cycle eventually exhausts the supply of nutrient and oxygen from the external environment. Accordingly, the embedded cells suffer from the lack of nutrients, creating a bottleneck for the growth of thick (e.g. >1 mm scale) 3D tissues. Studies (Sutherland, R. M. et al., Cancer Res. 46, 5320-5329,1986; Martin, et al., Ann. Biomed. Eng. 27, 656-662, 1999) confirm that the cells in the tissue are poorly cultured when they are further than about 400 μm from the external nutrient source. As a matter of fact, in real tissue, almost all the cells are within 100 μm of a capillary vessel. Several authors tried to enhance the mass transport in tissue culture with different approaches. For example, Neumann et al. (Microvasc Res 2003; 66:59-67), insert and extract nylon strands and tubing to generate straight artificial blood vessels that are cultured to deliver the culture medium. However, no real vascular system is composed of straight capillaries and it is impossible to connected thousands of capillaries when transplanting in vitro tissue to host body. Griffith, et al (Ann NY Acad Sci 1997; 831:382-97) introducing a three dimensional printing process to create three dimensional channels, but the resolution of that technology is only 200 μm, which is far from capillary dimension (<10 μm). Another group (Borenstein, et al. Biomedical Microdevices 2002,4:3,167-175) used silicon microfabrication technology and molding to create two dimensional micro channels for enhanced mass transport. Nevertheless, the three dimensional nutrient transport in thick tissue culture remains a big hurdle in tissue engineering. The current state of micro vascular networks in tissue engineering is addressed in Ruben et al. Biomaterials 26 (2005), 1857-1875.

A novel three dimensional microfabrication technology, the Projection Micro-Stereolithography (PμSL) (Sun et al. Sensors and Actuators A, 121 (2005), 113-120), is introduced and coupled with mass transport simulation to the design and fabrication of vascularized micro bioreactors. The microfabricated bioreactor dramatically enhances the three dimensional mass transport by advection and diffusion through micro fabricated capillaries. This microfabrication method brings several unique advantages to the advanced microbioreactor research and development: first, the capability of PμSL to build truly 3D sophisticated microstructures with very fine spatial resolution at micron scale; second, a significantly shortened design cycle enabled by high fabrication speed (1000 layers in a couple of hours; finally, the choice of biocompatible and biodegradable polymers offers flexibility for fabricating implantable vascularised scaffold for different tissue culture (Ratner et al. Annu. Rev. Biomed. Eng. 2004. 6:41-75; Hou et al., Mater. Chem., 2004, 14: 1915-1923).

One of the obstacles of culturing functioning tissues using bioreactors is to obtain a substantial biomass (Berthiaume, et al, Tissue Engineering, Encyclopedia of Physical Science and Technology, 3d Ed., Vol 16; Martin et al. Trends in biotechnology. 22, 80-86, 2004). Most cell cultures produce flat, one-cell-thick specimens that are not appropriate for insertion into three-dimensional tissue spaces and also offer limited insight into how cells work together. With increased cell density, the metabolism during cell growth cycle will eventually exhaust the supply of nutrient and oxygen from the environment and the embedded cells suffer from the lack of nutrient, creating a bottleneck for the growth of 3D tissues. Studies confirm that the cells in the tissue are poorly cultured when they are further than ˜400 μm from the nutrient supply source. In order to enhance the mass transport, several designs of bioreactor have been proposed, such as spinner-flask bioreactors by stirring the medium; rotating wall vessel bioreactor invented by NASA to suspend the cells in a medium; hollow fiber bioreactor delivering nutrient through permeable fibers or in the opposite way by culturing cells inside the fiber, and direct perfusion bioreactor using porous scaffold to transport nutrients to the cell in the pores. However, the state-of-the-art bioreactors share some common drawbacks: most of the bioreactors are designed for culturing a few types of cells or cell groups at low cell density, and after the cells are cultured they have to be harvested and collected, losing the integrity of the tissue. In addition they are not compatible to fast throughput tissue assays to study the impact of local environment on a small volume of cells. Although there are some studies on the micro-bioreactor, for example, the micro-encapsulation immobilizing cells in a micro compartment, the nature of this method limits compartment geometries to very simple cases.

The majority of nutrient exchange takes place in capillary-sized vessels, having a diameter less than about 10 um and a wall thickness on the order of 1 um. In the microcirculation, for example, diffusion of materials from the blood vessel to the surrounding tissue (and vice versa) takes place in the capillaries and post-capillary venules. There is a need in the art to provide efficient systems and methods capable of reproducing an in vivo microvascular networks, including networks whose permeability to a biological material varies along the network tree. Such networks are useful in a number of applications including tissue implants, providing cell expansion, bioreactors, and a number of systems for the production of biological materials and biofuels.

### SUMMARY OF THE INVENTION

The present invention relates to methods and systems for generating three-dimensional networks of microvessels. In particular, the microvessel networks generated by the present invention are uniquely capable of sustaining cells in a similar manner to the microvasculature in the body facilitating nutrient and waste exchange, thereby supporting the surrounding tissue. The three-dimensional aspect of microvascular networks made by the systems presented herein provide the capacity to feed and drain small-diameter vessels in a network by a feeding and collecting vessel, respectively. These methods permit the systems presented herein to feed and support potentially large tissue volume regions within a bioreactor, thereby facilitating growth and expansion of cells. The three-dimensional microvascular network may be imbedded within a bioreactor. Such bioreactor systems are particularly useful in generating tissue implants or producing useful materials such as bioactive agents, biofuels, drugs and any number of a wide range of useful cellular-generated materials.

Furthermore, microfabrication techniques presented herein can directly fabricate three dimensional geometric structures and microstructures (e.g., having at least one dimension that is less than 1 mm) that is not attainable with conventional microfabrication techniques, including overhang and movable microstructures. The advantages of the microstructure network generated by processes provided herein include the parallel process nature of the network generation which yields high speed network generation, high resolution (e.g., better than 2 μm) and it is readily scalable from the micrometer scale to the macro scale (e.g, greater than 1 mm). For example, the fabrication area can be on the order of 40 mm by 40 mm or greater, with fabrication speeds of about 180 layers/hour (corresponding to about 6 mm³/h). In addition, any network geometry can be made, including geometries that are inherently fragile, by providing sacrifice elements that temporarily support the fragile microstructure and are subsequently removed after processing.

In an embodiment, any of the methods and systems disclosed herein relate to a microvascular network having a permeability or diffusivity to a biological material that varies with location within or along the network. The ability to selectively adjust permeability over the vascular tree geometry presents a number of advantages. First, by minimizing initial diffusion of biological material, nutrients or required metabolites out of or into the feed vessel, the concentration gradient between the nutrient in the lumen of the capillary and surrounding tissue is maximized. Increased concentration gradient facilitates increased diffusion of the material from the lumen to the tissue, for example, thereby increasing the effective volume that can be fed by an individual vessel.

An aspect of the invention provides the ability to generate a microvascular network with a selectively-controllable permeability by projection micro-stereolithography (PμSL). For example, the vascular permeability may be varied along the vascular tree, thereby ensuring that most nutrient diffusion from the vascular lumen to the surrounding tissue occurs in a region corresponding to the in vivo capillary network. In an embodiment, the permeability is tuned to a specific material of interest, thereby maximizing the diffusivity of the material through the vascular wall in a localized region. For example, if certain cells or materials (e.g., drug-containing materials) are spatially distributed, the permeability of the vascular tree may be correspondingly spatially distributed to facilitate maximum exchange of material. In an embodiment, tuning is accomplished by optimizing the degree of cross-linking within the network wall with the size of the biological material of interest (e.g., less cross-linking for larger molecules).

In an embodiment, the present invention provides methods for making a microstructure such as networks of interconnected microvessels or microvascularized bioreactors. A photocurable liquid composition having a top surface is illuminated with a light source that generates a pattern of electromagnetic radiation encompassing or having a wavelength that is capable of curing at least a portion of the composition. In particular, the top surface of the composition is illuminated in a pattern to generate a corresponding pattern of polymerized composition having a layer thickness. This polymerized layer is immersed in the liquid composition by a vertical displacement, where the magnitude of the displacement corresponds to the layer thickness. As known in the art, layer thickness may be manipulated by selection of one or more of illumination intensity and/or time, liquid composition, or use of material that absorbs light (e.g., photoabsorbers). To ensure maximum resolution in the generated pattern, in an embodiment there is a delay between after immersion of the polymerized layer and subsequent exposure of another light pattern illumination of the liquid composition. This delay time is called a “surface dwell time” and ensures that the liquid composition top surface becomes substantially level. The value of the surface dwell time impacts the time required to generate the vascular network and depends on a number of physical parameters including liquid viscosity, layer thickness, and the speed of the vertical displacement of the polymerized layer. The exposing step is repeated any number of times as desired depending on the final dimensions (e.g., length or width) of the vascular network, with each exposing step capable of independent light pattern exposure to generate an adjacent polymerized pattern layer. By contacting consecutive and adjacent polymerized pattern layers, a microvascular network having an interior surface and an exterior surface is obtained. After optional processing of the polymerized network (e.g., washing), the exterior surface is contacted with a cell population, to obtain a microvascularized bioreactor.

In an embodiment, the microvascular network has a tunable permeability to a biological material that varies with the location in the network. The biological material may be supplied by the user, such as by the flow of culture media through the lumen of the vascular network. The biological material may be produced by at least a portion of the cell population, such as by cells that have been bioengineered. In an embodiment, the system is tuned to a plurality of biological materials.

In an aspect, the desired microvascular network is digitally stored in a computer. The network is then divided into a plurality of adjacent layers, each having a layer thickness and each pattern of illumination corresponds to a layer stored in the computer and is provided to the photocurable liquid composition.

In an embodiment, permeability of the network is spatially varied by gray scale illumination. Gray scale illumination is by any means known in the art, such as by a gray scale mask capable of achieving a plurality of illumination intensities, such as a continuous or non-continuous illumination intensity selected over an intensity range ranging from zero to a maximum level.

In an aspect, gray scale illumination is provided in at least one illumination step. The gray scale may be achieved by providing by a gray scale mask (e.g., a digital mask) having a plurality of pixels, each pixel capable of providing a plurality of grayscale shades each having a unique intensity. Such a digital gray scale mask may be connected to a computer that has stored a plurality of layers to be polymerized. In an aspect, the number of grayscale shades is between 8 and 512. There are two types of gray scale systems and each depend on whether the gray scale minimum illumination intensity is sufficient to generate polymerization resulting in change of state from liquid to solid. For example, a minimum illumination intensity sufficient to cause polymerization generates a polymerized layer having uniform height (e.g., layer thickness), but with variable cross-linking, thereby providing variable permeability in said polymerized layer. For example, a cross-section of a vessel wall, in this aspect, has a uniform and continuous wall thickness. The permeability, however, of that cross-section varies due to the spatial variation in cross-linking.

Alternatively, the gray scale method may provide a gray scale minimum illumination intensity insufficient to generate polymerization. In this aspect, the gray scale illumination provides a means for generating solid features within a layer, each feature capable of having a distinct and different height. This capability facilitates making a continuously changing surface profile that is smooth and/or discontinuous surface profile having straight-edged features (e.g., 90° angle of feature edge relative to underlying substrate). In addition, this type of gray scale processing provides a means for defining regions of the fabricated feature having relatively low cross-linking density that can be optionally dissolved in subsequent steps. An example of an application where such a property is useful is in the design and fabrication of well-defined time-release of biological agents that are impregnated or contained within such features.

Another embodiment provides microstereography methods capable of significantly reducing surface dwell times by electrowetting. The electrowetting uses a two-immiscible fluid interface system that is operably connected to the liquid top surface, with one fluid that is conductive and the other fluid that is non-conductive. A voltage is applied to the conductive fluid to flatten the interface, thereby decreasing the surface dwell time. Without this electrowetting step, the dwell time is significantly longer as the liquid top layer relaxes back to a substantially flat state under the influence of gravity only after being disturbed by immersion of the polymerized layer. In an aspect, the two-fluid system is a conductive, salted polyethylene glycol diacrylate (SPEGDA) fluid on the bottom adjacent to the top surface of the photocurable polymer, and nonconductive octane fluid on top. The immiscible top liquid may also optionally include, but is not limited to: bromodecane, toluene, chloroform, dibutyl-ether, dodecanethiol, etc. In an embodiment, the electrowetting reduces surface dwell time by at least 20%, or between about 20% and about 70%, compared to a system without electrowetting, with an attendant significant reduction in the time required to generate a vascularized bioreactor.

In another embodiment, the invention provides a capability to accurately and rapidly reproduce three-dimensional in vivo microvascular networks. For example, an in vivo vascular network, including a microvascular network, is captured in silico (e.g., digitally stored), and that information used in any of the methods for generating a vascularized bioreactor. The captured network image is divided into a plurality of adjacent layers, with each layer corresponding to an illumination pattern that polymerizes a distinct layer on the surface of liquid photopolymer. Sequentially patterning the layers provides a microvascular network made from the photocurable liquid and having a geometry corresponding to the in vivo microvascular network.

The microvascular networks generated by the methods and systems disclosed herein have any number of controllable parameters. For example, the bioreactor fed by the manufactured network can have a wide range of tissue volumes, such as a range that is selected from between 0.1 μL and 1000 mL, or between about 0.1 μL and 80 mL. Depending on both the size of the bioreactor, and the desired resolution of features in the vascular network that feeds the bioreactor, the total number of layers is selected from a range that is between 100 and 3000. The system provides the capacity to select layer thickness, such as a thickness that is selected from a range that is between 0.1 μm and 50 μm. The wall thickness of the smallest microvessels in the network is preferably thin to maximize diffusion and permeability of the wall, such as a thickness that is between 1 μm and 20 μm. A practical limit as to the smallest wall thickness is related to the fragility of the network, with very thin walls being prone to fracture and breakage. Another parameter of interest is the capillary density or the number of vessels in a unit area. For resource-intense applications, where maximum diffusion is desired, the capillary density is selected to be high, such as from a range that is between about 50 per mm² to 150 per mm².

The methods and systems of the present invention are optionally described referring to functional parameters, such as being capable of expanding a seeded cell population or providing 10⁷ to 10⁸ cells/mL of cells that are capable of making a biological material or precursor thereof. For example, the bioreactors of the present invention are optionally capable of expanding a cell population by at least 4-fold, or a range selected from between 2 and 20-fold. This is particularly useful in situations where the bioreactor is to be implanted into a patient suffering a tissue defect, and either source cells that make up the tissue is in short supply or the patient's own cells are to be used and it is desired to minimize the number of cells obtained directly from the patient.

In another embodiment, tuning a physical parameter within the network is accomplished by gray scale processing. In an aspect, the physical parameter is selected from a parameter that directly affects the ability of a material to diffuse across the vessel wall, such as wall thickness, diffusivity or permeability. Alternatively, the parameter may be one that affects a mechanical property, such as Young's modulus, density, compressibility, bending modulus, as well as swelling sensitivity in different pH levels.

In an aspect, any of the methods or devices provided herein vary or select permeability by generating pores in the vessel walls of the microvascular network with a size that is greater than or equal to 20 nm and less than or equal to 1 μm.

In an embodiment, any of the methods provided herein use gray scale illumination to generate a polymer pattern having a spatially varying cross-linking density or spatially varying etch rate when exposed to an etchant. For example, any of the illuminating steps provided herein optionally comprise illuminating a first region with a first light exposure, and illuminating a second region with a second light exposure. In an aspect light exposure refers to illumination intensity, illumination duration, or both. The second light exposure can have an intensity or duration or both that is less than the first light exposure intensity, duration, or both. In this manner a polymer is generated in the first region that has a cross-linking density that is greater than the second region cross-linking density.

In an aspect, intensity, duration or both of illumination are selected such that an etch rate of the first region polymer and the second region polymer when exposed to an etchant is at least about 5 to about 10 times different from each other. Although the methods are compatible with any specific etch rate, in one embodiment the etch rate in the first region having a higher cross-linking density than the second region is greater than or equal to 160 μm per hour.

In this aspect, the first region polymer is optionally a microstructure that is a part of the microvascular network and the second region polymer is a sacrificial element. “Microstructure” refers to an element having at least one dimension that is less than 1 mm and that is used in the final generated polymer that forms a network part of a bioreactor or other device. In an embodiment, the sacrificial structure is made from a material that is the photocurable liquid composition that generates the polymerized pattern layer, thereby providing simultaneous generation of both sacrificial element and microstructure. Optionally, any of the methods provided herein undergo another step of contacting the sacrificial element with an etchant to at least partially or to completely remove the sacrificial element. Although the methods provided herein are compatible with any photocurable liquid composition and etchant that etches the resultant polymer, one embodiment provides a photocurable liquid composition that is 1,6 hexanediol diacrylate and an etchant that is an acidic solution such as sulfuric acid and hydrogen peroxide or water soluble photopolymer (e.g., epoxylated (30) bisphenol a dimethcrylate) and water. An optional step is a preliminary washing step with a good solvent of the monomer solution to remove unpolymerized monomer, such as contacting with acetone, for example.

The processes provided herein are not limited to making microvascular networks, but are compatible with generation of any geometry or device, such as devices requiring complex three-dimensional microstructures and patterns thereof including but not limited to bioreactors, MEMS, microfluidic devices, microstructures having movable microcomponents and overhang microstructures, such as a microfluidic device having one or more elements capable of sensing and actuation, heat exchangers and actuators. In addition, the device may be a reservoir of a biologically-active material that is desired to be released to surrounding tissue to promote one or more biological events. For example, the device can be a reservoir of growth factor or related regenerative medicine for promoting tissue growth and/or repair.

In an embodiment the inventions is a method of making a three-dimensional device by providing photocurable liquid composition having a top surface. A light source is provided that is capable of curing at least a portion of the photocurable liquid composition when the top surface is illuminated. Optionally, the illumination of the top surface is with gray scale illumination, wherein the gray scale illumination is a pattern of light intensity or duration that generates a pattern of polymer having a spatially varying cross-linking density. The gray scale illumination is by any means known in the art such as by a digital mask like an LCD. The polymer pattern is contacted with an etchant that selectively removes polymer having a lower cross-linking density to make the resultant three-dimensional device.

As described herein, multiple illumination steps provide a bottom-up processing and development of structures made of layers separately illuminated. In an aspect, the device structure is made from a plurality of polymer layers by illuminating the composition top surface with the light source, wherein the illumination is in a pattern thereby simultaneously generating a polymerized pattern layer having a layer thickness. The polymerized pattern layer is immersed into the composition depth by a vertical displacement corresponding to the layer thickness. Waiting a surface dwell time allows the surface to become substantially level and the illuminating step is repeated any number of times as desired to generated adjacent and consecutive polymerized pattern layers. In an aspect of this process, at least one element is a sacrificial element having a lower cross-linking density that supports at least a portion of said three-dimensional structure during processing. In particular, the structures of the device made by the present processes have a higher cross-linking density, and therefore, a lower etch rate than the sacrificial structure. Certain patterns and microstructures in the device, however, are inherently fragile and difficult to make owing to their small size and geometrical connections (e.g., overhang structures or orientations that are not inherently well-supported by surrounding elements) and often break, fracture or otherwise deform during handling or subsequent. Incorporation of sacrificial elements that support the fragile portions of the device's structure maintains the geometry during subsequent processing and handling steps and can be finally removed to ensure the device's desired structure is maintained. In an embodiment, the device can be manufactured and shipped to an end-user with the sacrificial elements intact, and a kit with instructions provided to the end user for removing the sacrificial element(s) prior to use of the device.

In another aspect, the gray scale illumination is selected (e.g., either intensity, duration or both) to generate a first region of polymer that is a sacrificial element and a second region of polymer that is a structure or a microstructure, wherein the sacrificial element provides physical support to the structure or the microstructure. The sacrificial element that supports a structure such as a microstructure is optionally described in terms of a functional parameter, such as an etch rate. Similarly, the structure or microstructure that is to form a part of the device is optionally described in terms of an etch rate. In an embodiment, the sacrificial element has an etch rate that is at least 5-10 times greater than etch rate of fully crosslinked microstructure. Any of the methods described herein can be used to generate a microstructure that is an overhang structure or a movable element. “Overhang structure” refers to an element made by a process provided herein that is part of a device network pattern that does not have an underlying supporting substrate. Optionally, the overhang structure is supported by polymerized layers above the overhang structure or horizontally-oriented (including structures oriented to run in a vertical direction, as summarized in FIG. 72A).

Another useful embodiment of the invention is a method of producing a biological material by providing a vascularized bioreactor having a three-dimensional network of microvessels capable of fostering a cell population. The network is optionally produced by any of the methods of the present invention to provide a network having a polymeric wall with a lumen-facing side and a cell-facing side. The wall is configured to have at least a portion that is permeable to the biological material, and where the wall permeability spatially varies in the network. A culture media capable of fostering cell growth is introduced to the network at an inlet port upstream of the network. The media exits the network at an outlet port downstream of the network and is collected. A cell population capable of producing the biological material or a precursor thereof is contacted with at least a portion of the outward-facing side of said network wall and the cells are cultured in the bioreactor by introducing a culture media to the inlet port, thereby exposing the network wall to the culture media. Diffusion of needed raw material to the cells, and removal of both desired biological material and unwanted cellular metabolism byproduct occurs across the vessel wall. The introduced cell population produces a biological material capable of diffusing from the cell population to the culture media through the network wall so that the collected media step collects at least a portion of the produced biological material. Such a system is capable of manufacturing large amounts of a biological material in a cost-effective and efficient manner. In an embodiment, the biological material is a biofuel, pharmaceutical such as an antibiotic or protein, drug, a prodrug, or any precursors thereof. The biofuel, in an aspect, is ethanol, such as ethanol produced by a yeast cell. Other biofuels, such as butanol and lactic acid can be harvested and purified using any of the bioreactors disclosed herein. In another aspect, the bioreactor is a continuous-flow system wherein culture media is continuously flowing through the network, such as without intervention of the fermentation steps.

In an embodiment the network of microvessels has a first portion that is substantially not permeable to the biological material and a second portion that is substantially permeable to the biological material. This can be useful when it is desired to maximize the concentration difference across the vessel wall to particular regions of the network to increase collection of biological material, for example. In this context, a first portion wherein the permeability of the material is less than 2% the permeability of the other portion, is said to be not permeable relative to the other portion. Any of the microvessels have a selected dimension. For example, in an aspect the length is between about 200 um and 10 mm; the inner diameter is between 10 um and 60 um; or the vessel wall thickness is between 5 um and 20 um.

In an embodiment, any of the polymeric wall permeability is spatially varied by varying the amount of cross-linking in the vessel wall.

Bioreactors produced and disclosed herein have a range of utility, including as implants for delivery of a material to the body, tissue implants, for the manufacture of pharmaceuticals or biofuels. Depending on the desired use of the bioreactor, the relevant substance in contact with the microvascular network is chosen accordingly. For example, if the bioreactor is for the manufacture of a material used in making a drug, the substance may comprise a cell bioengineered to overexpress that material. If the use is for in vivo delivery of a material, the substance can be a matrix impregnated with the material that provides time-release dosing. A bioreactor for implantation into a patient optionally comprises a cell population derived from the patient to minimize potential immune-response activity by the patient post-implantation.

In an embodiment any of the devices and networks provided herein and made by any of the disclosed methods are used to calibrate a medical device, such as a medical device that images a biological tissue to detect a disease state. In particular, a microvascular network that supports a cell population can be used to calibrate the medical device by imaging the artificial microvascular network with a medical instrument to obtain output data and calibrating the medical instrument with the output data. “Output data” refers to one or more values that are used to detect a disease state such as intensity from a signal, resolution, image appearance, morphology. In an aspect, the medical device images by a technique such as magnetic resonance imaging, ultrasound, computed tomography, fluoroscopy, radiography, thermography or positron emission tomography. The microvascular network made by a process disclosed herein can model one or more of a biological tissue or a disease state. For example, for various cancer detection models, a tumor may be supported by the network. Similarly, depending on the tissue of interest, an appropriate network of vessels is formed by the artificial network and is used to support appropriate cell and tissue type (e.g., bone, brain, breast, liver, pancreas, blood vessels, lymphatic vessels).

In an aspect, the calibration further relates to introducing a challenge to the network. “Challenge” is used broadly to refer to any physical or biological introduction that models a biological condition. For example, for cardiovascular disease states that relate to physical or geometrical change, thereby producing one or more symptoms, the challenge may relate to a change in the vessel geometry or patency. Similarly, for cardiovascular disease states related to a stenosis, restenosis, plaque build-up on the luminal-facing side of the vessel wall, an obstruction or narrowing may be introduced to a vessel within the network. Some examples of challenges include, but are not limited to an at least partially obstructed vessel, a vessel geometry that models a cardiovascular disease, wherein the cardiovascular state is a blood vessel having an aneurysm, atherosclerosis, blood vessel wall thickening, blood vessel wall hardening, or blood vessel leakage, and an at least one cell type that models a disease state. Examples of vessel geometry that models a cardiovascular state includes, but is not limited to, varying wall thickness, elasticity, porosity, patency (e.g., introducing tears or holes through the wall), variations in vessel wall geometry such as ballooning of the vessel wall in the case of aneurysm models or infiltration of the lumen by the vessel wall (to model plaque-build up such as occurs during atherosclerosis). With respect to a biological challenge, a specific cell type may be introduced to the system, such as a cancer cell line or tumor to assist in calibrating the instrument for cancer-cell or tumor detection, for example. Optionally, the challenge is introduced during real-time imaging, such as introducing a clot, clog or broken vessel.

Without wishing to be bound by any particular theory, there can be discussion herein of beliefs or understandings of underlying principles or mechanisms relating to embodiments of the invention. It is recognized that regardless of the ultimate correctness of any explanation or hypothesis, an embodiment of the invention can nonetheless be operative and useful.

### BRIEF DESCRIPTION OF THE DRAWINGS

FIG. 1 Serial Stereolithography Configuration.

FIG. 2 Projection Stereolithography Configuration (PμSL).

FIG. 3 Microstructures Fabricated with Projection Microstereolithography A. Micromatrix with 150 μm grid dimension and 20 μm line width (scale bar 200 μm). B. Freestanding polymer micro-network at UIUC with pores of diameter as small as 2 μm at spacing down to 2 μm (scale bar 20 μm).

FIG. 4 Four Views of Microbioreactors.

FIG. 5 Microtubule network.

FIG. 6 Perspective View of Bioreactor having a plurality of parallel microvessels.

FIG. 7 Substrate holder capable of vertical displacement to immerse polymerized layers.

FIG. 8 Microstereolithography Stage Motion identifying position during illumination (A); after polymerization of initial layer (B); lowering and raising of stage (C, D); position and substantially flat liquid surface ready for illumination corresponding to next adjacent layer polymerization.

FIG. 9 Bi-Layer Dielectric

FIG. 10 Conductivity Measurement Set-Up.

FIG. 11A Conduction in PEGDA-258. B Conduction in PEGDA-258A+NaCL

FIG. 12-Conduction of PEGDA-258+ Imidazole Trifluoromethanesulfonate Salt at 0.3%, 1.0% and 5.0% concentration.

FIG. 13 Hanging Droplet for Surface Tension Measurement.

FIG. 14 Curve Trace for Surface Tension Measurement.

FIG. 15 Single Droplet on Teflon for Contact Angle Measurement.

FIG. 16 Curve Trace with Tangent Lines for Contact Angle Measurement.

FIG. 17 Pendant prop Variables.

FIG. 18 Goniometer Profiles of Electrowetting Fluids: A Goniometer Profile: DI Water; B Tap water; C Octane; D SPEGDA-258; E SPEGDA-258A; F SPEGDA-575; G SPEGDA-6000.

FIG. 19 Single Droplet Electrowetting Setup.

FIG. 20A Electrowetting Contact Angle Response of SPEGDA-258A. B SPEGDA-258A 0V; C SPEGDA-258A 150V.

FIG. 21A Electrowetting Contact Angle Response of SPEGDA-575. B SPEGDA-5750V; C SPEGDA-575 150V

FIG. 22 A Electrowetting Contact Angle Response of SPEGDA-6000. B SPEGDA-60000V; C—SPEGDA-6000 150V.

FIG. 23 A Electrowetting Contact Angle Response of tap water B tap water 0V; C tap water150V.

FIG. 24 Two Fluid Electrowetting Setup: A Front View; β Isometric view.

FIG. 25 Two Fluid Electrowetting Setup at “Flattened” Voltage: A Front View; β Isometric view.

FIG. 26 Two Fluid System at: A 0V; B 150 V. Close-up view of left (C) and right (D) at 150 V.

FIG. 27 Optical Set-Up for Two Fluid Electrowetting Microstereolithography.

FIG. 28 Enhanced PμSL Multilayer Process Steps via electrowetting.

FIG. 29 A pattern used as bitmap mask.

FIG. 30 Comparison of corresponding A image generated with (A) and without (B) electrowetting.

FIG. 31 Electrowetting Sample Close Up: Mag 20×.

FIG. 32 Three bar pattern.

FIG. 33 Sputtered Housefly Wing Mounted Upside Down.

FIG. 35 Reconstructed Cross-Sectional Image Slice from Sagittal Image Scans.

FIG. 36 Amira Isosurface Model from CT Scan of Common Housefly Wing.

FIG. 37 PEG Model of Flywing.

FIG. 38 Main Flywing Tubule.

FIG. 39A Cross-Sectional Image Slice Reconstructed from Sagittal Image Data. B Enlarged Area; C Bottom layer; D Mid layer; E top layer.

FIG. 40A Flywing Main Microchannel Model; B Top view.

FIG. 41A Grayscale bitmap; B Black and white (BW) (e.g., all or none) bitmap; C Grayscale Height Profile (Side View); D BW Height Profile (Side View).

FIG. 42A Grayscale Bitmap; B Height Profile (Side View); C Crosslinking Density.

FIG. 43A Grayscale Contact Lens Surface; B Test Bars for Dₚ Measurement.

FIG. 44 Dp Measurement Setup.

FIG. 452D Oxygen Diffusion Pinwheel Dp=39 μm.

FIG. 462D Pinwheel Embedded in 3D Cylinder.

FIG. 47 Pinwheel at t=0 s.

FIG. 48 Center Close View at t=100 s.

FIG. 49 Surface Plot of Confocal Image in FIG. 54.

FIG. 50 Data Extraction Axes.

FIG. 51 Major Diagonal Isometric Plot.

FIG. 52 Radial Plot of Grayscale Intensity: Sections 4 and 8.

FIG. 53 Fluorescent Image of Injection Hole at t=10 sec.

FIG. 54 Grayscale Image of FIG. 59 with Axis Rays.

FIG. 55 Grayscale Intensity vs. Radial Position with Optimal Diffusion Solution—Axis 1: D=73 μm²/s.

FIG. 56A Axis 1; B Axis 2; C Axis 3; D Axis 4; E Axis 5; F Axis 6; G Axis 7; H Axis 8.

FIG. 57 Diffusion Coefficients Along Eight Matrix Axes at t=10 s.

FIG. 582D Pinwheel Cross Diffusion.

FIG. 59 Fluorescent Image of 2D Pinwheel Injection Hole at t=5 sec.

FIG. 60 Diffusion Coefficients Along Eight Matrix Axes at t=5 s & t=10 s.

FIG. 61: 3D micro bioreactor fabrication via layer-by-layer photo-polymerization of a biocompatible monomer, according to the slicing of the 3D computerized model.

FIG. 62 contains four electron micrographs showing examples of high resolution 3D microfabrication by PμSL. A is a perspective view of microvessel networks fed by a common vessel. B is a top view of the network shown in A. C is a close-up view of the network in D through the window, illustrating another network can comprise a plurality of substantially parallel microvessels.

FIG. 63 Experimental data of effective ethanol diffusion coefficient as a function of UV exposure time in 200 μm thick PEG (MW575) film, T=20° C. UV source: λ=390 nm, 1.1 mW/cm².

FIG. 64: A, B, D different views of Micro bioreactor; C, yeast cell culture device, the culture medium is perfused from external pipe through the polymer capillaries in the micro bioreactor. The bioreactor is submerged in DPBS. When the culture medium flows through the capillaries, it diffuses from the interior of the capillary to the exterior through the capillary wall. The glucose metabolism of yeast cells will produce ethanol. It will diffuse into the DPBS solution in the culture chamber where it can be collected or permitted to diffuse into the capillary lumen and convected from the system at a downstream outlet port by the flow of culture medium.

FIG. 65 Computational experiment illustrating the minimum D-Glucose concentration in a bioreactor increases as the inner radius of the capillary increases. Capillary center to center spacing=120 μm, the capillary wall is 10 μm thick

FIG. 66 Two experiments based on yeast model were conducted to verify the simulation summarized in FIG. 71. In A, the inner radius of capillary is 30 μm, the cracks are due to the collapse of the capillary during the sample drying process. In B, the inner radius of the capillary is 20 μm. The capillaries collapsed during drying process. The inset in each of the panels is a close-up view of the indicated region and the scale bar is 5 μm and 10 μm for A and B, respectively.

FIG. 67 is a graph showing the average increasing rate of glucose concentration of the DPBS in the reaction chamber of FIG. 72A. In the control experiment, no yeast is seeded in the bioreactor. The same culture medium is pumped through the capillary and the glucose concentration in DPBS is measured after specified time periods.

FIG. 68 Schematic drawing of fabricating “ceiling lamp” and moving part with sacrificial structure in PμSL. In step 1, the designed micro structures are fabricated using PμSL. The sacrificial structures are polymerized using lower grayscale which results in a lower degree of polymerization. In step 2, the sacrificial structure is preferentially etched away (due to faster etch rates arising from lower degree of polymerization) and releases the hang over structure and moving part. The arrow indicates the fabrication direction. (e.g., bottom up layer deposition).

FIG. 69, A: Hair tree after acetone treatment but before acid etching, part of the sacrificial structure has been removed by acetone. B: Side-view of hair tree after acid etching. C: Top-view of hair tree after acid etching. D: Side-view of hair tree after acetone treatment but without using sacrificial structure.

FIG. 70 A: experiment data of etching rate under different irradiation time, grayscale of mask is 255. B: experiment data of etching rate under different irradiation light intensity controlled by grayscales of digital image.

FIG. 71A Network of microstructures that are micro capillaries the from a microvascular network generated from 250 layers. B Dense capillary network made from microstructures that are cylindrical tubes having an inner diameter to 20 μm, an external diameter of 40 μm and a length of 800 μm. C A micro cake having dimensions of about 2.5 mm×2.5 mm×2 mm. D Microstructures that are hair cells that can be used for flow sensing.

FIG. 72 Schematic illustration of the drawbacks of conventional PμSL and other microfabrication techniques. A and B define an angle θ corresponding to a branch angle during bottom up layer by layer microfabrication (as indicated by the direction of the arrow). “Limited 3D” is defined for situations corresponding to A, where PμSL are able to generate branches for θ>90°. Limited 3D techniques cannot satisfactorily generate structures where the angle θ≦90°, whereas a “full 3D” PμSL process can (as illustrated by B). C shows a conventional limited 3D PμSL process wherein for θ>90° the branches are satisfactorily produced, but for θ≦90° the branches sag. D schematically illustrates a full 3D microstructure technique that generates satisfactory structures for both θ≦90° and θ>90° via incorporation and subsequent removal of sacrificial structures.

FIG. 73 Schematic illustration of one method for generating full 3D structures and an advantage of using a digital mask (left columns) compared to a physical mask (right column. A Graphical illustration of illumination of a digital mask (left) and a physical mask (right). B illustration of a plot of grayscale intensity of a digital mask (left) and a physical mask (right). C Graphical illustration of etch rate as a function of illumination intensity.

FIG. 74 Schematic illustration of etching kinetics for photopolymerized polymer exposed to different light intensities and/or exposure times.

FIG. 75 Vascularized bioreactor where nutrients and waste products pass through the microvessel wall.

FIG. 76 CHO cell perfusion culture for an initial seeding of about 60 CHO cells followed by fluorescent imaging after 6 days of culture. A shows a top view; B shows a side view. Control views correspond to experiments performed with stationary buffer solutions, without perfusion of culture medium after cell seeding.

FIG. 77 Images demonstrating controllable porosity of networks, where pore size and density are controlled.

FIG. 78 Illustration of a prostate cancer model using a microbioreacter generated by a process disclosed herein. A Seeded cells that are fibroblasts; B seeded cells that are epithelial cells; C Histological image of the bioreactor, with fibroblast cells cultured around the microvessels.

FIG. 79 A three-dimensional microcapillary system for tissue engineering. A provides a schematic illustration of a cell population supported by media flowing through the artificial microvessel. B shows a porous scaffold. C is an illustration of a microvascular network example having a geometry that the disclosed process can use as a model.

### DETAILED DESCRIPTION OF THE INVENTION

“Microvascularized bioreactor” refers to a system that supports biological material such as cells and tissue in a manner similar to how blood flow within the vasculature supports surrounding cells in vivo. In particular, a microvascularized bioreactor refers to a three-dimensional microvascular network in which a fluid medium flowing in the network of vessels is capable of supporting a cell population. “Microvascular” refers to a vessel having a lumen diameter on the order of less than 1 mm, or 500 μm or less, or 100 um or less, and preferably about 10 um or between 8 um and 25 um. The network can comprise a tree of microvessels (e.g., a single inlet that bifurcates along the tree into smaller vessels and then at a distance along the tree the multiple vessels rejoin into larger size vessels into a single collection vessel) whose dimensions or diameter depend on the longitudinal location along the tree, such as feed vessels on the order of mm scale and the smallest capillaries on the order of 8 to 25 μm diameter scale.

“Photocurable liquid composition” refers to a liquid capable of undergoing polymerization in response to electromagnetic radiation, such as by ultraviolet (UV), visible or infra-red illumination. “Curing” refers to the polymerization of a portion of the liquid such that a portion of the liquid is solidified while other portions remain in a liquid state. “Pattern” refers to the source of light that is applied with a magnitude of illumination that varies with surface location. The pattern may be a simple “black and white” or on/off pattern (either illuminated or not illuminated). Alternatively, the pattern may be a gray-scale pattern, where the illuminated intensity is capable of more than the simple two values state of the on/off pattern. In particular, gray scale illumination provides the capability of spatially varying polymer cross-linking density, thereby generating sacrificial elements and microstructures, as well as providing permeability control. “Sacrificial element” refers to any polymerized polymer that can be subsequently preferentially removed in one or more processing steps without destroying or adversely impacting a corresponding microstructure that may be supported by the sacrificial element.

“Layer thickness” refers to the depth of the polymerized pattern. The interaction of the illumination pattern with the liquid surface provides for polymerization that tends to occur beneath the exposed surface. The depth of this polymerization may be controlled to provide for layers that are thinner, thereby providing finer control of the generated network. The trade-off, of course, is the increased number of steps required to generate the network and corresponding increase in production time. One means for controlling the depth of the layer is by adding material that tends to absorb the illuminating radiation, thereby decreasing the effective penetration depth of the radiation and reducing layer thickness. In addition, the magnitude of illumination may be varied.

“Immersing” refers to moving the illumination-induced polymerized pattern layer at the liquid surface to beneath the surface so that liquid is ready to receive a second illumination pattern to form a second polymerized pattern layer adjacent to the previous layer that has been immersed. Typically, a polymerized layer is immersed by a vertical displacement that corresponds to the thickness of the layer, or the thickness of the next to-be-polymerized layer.

“Surface dwell time” refers to the length of time between layer immersion and the subsequent introduction of the pattern of illumination to polymerize the next-adjacent layer. “Substantially level” refers to a surface that has a maximum height variation of less than about 1 μm, or that is less than 500 nm.

“Interior surface” refers to the lumen-facing surface of the vascular wall. “Exterior surface” refers to the outer surface of the wall that faces the cells that are to be supported by media flowing in the lumen defined by the interior surface.

“Contacting” the exterior surface with a cell population refers to placing cells within the bioreactor in “diffusive communication” with the vessel lumen. Diffusive communication refers to a biological material that is capable of diffusing from one side of the vessel wall to the other. For example, a material that diffuses from the lumen of the vessel to a cell that is located outside the vessel. Similarly, the term may refer to a material that is located outside the vessel, such as a material produced by a cell or material placed outside the vessel being capable of diffusing across the vessel wall to the interior (e.g., lumen).

“Biological material” is used broadly to refer to a material that is made by a cell or is desired to be introduced to the cell. The biological material may itself be useful as a therapeutic or used in the manufacture of a therapeutic. In an aspect, the biological material is capable of diffusing across the vascular wall of the bioreactor network system. Similarly, a biological material produced by the cell may be a material suitable in range of downstream applications, including industrial processes, such as a fuel component (ethanol, for example).

Permeability relates to the molecular transport of a material through the vessel wall. If the flux per unit area of a material (M) across the wall is J when the concentration difference across the wall is ΔC_{M}, then:

J=P_{M}*ΔC_{M}, or expressed as total flux:

J_{M}=P_{M}A*ΔC_{M},

Where P_{M} (in cm/s) is the vessel wall permeability to material (M), A is the effective surface area of the vessel wall through which flux occurs. Permeability is a mass transfer coefficient and is similar to the diffusivity (cm²/s) of a material:

$$J = - {D}_{A} \frac{d {C}_{A}}{d x}$$

(Fick's Law, D_{A} is the diffusivity or diffusion coefficient of the material (A).

Accordingly, permeability provides a measure of how readily a material can diffuse across a barrier, such as from within the vessel lumen to the surrounding tissue (or vice versa), and, as discussed below, may be used to ensure most or all cells within a bioreactor are well supported by culture media flowing within the vessels. Permeability may be computationally or experimentally measured by any number of methods known in the art, such as by measuring concentration in the vessel and outside the vessel, determining the concentration gradient across the vessel wall, and monitoring flow-rates in the vessel. The interplay between these variables determine whether the material is flow limited or diffusion limited and provides information necessary to determine optimal concentration and flow-rates in the vessel, vessel geometry, spacing and density, and cell number and distance from vessels. “Optimize diffusion” is used herein to refer tot selecting the permeability of a wall to maximize diffusion of a material while ensuring a cell population that is supported by the network remains viable. In an example, optimize diffusion refers to selecting permeability across the network so that diffusion is maximized within a particular region (such as in the smallest microvessels).

A material of interest in terms of permeability includes a nutrient. Nutrient is used broadly to refer to a substance required to support, or substantially assist, cell growth and maintenance. Accordingly, the term encompasses materials such as gases (e.g., oxygen), proteins, sugars, etc. Another material of interest in terms of permeability are materials that are produced or generated by cells that are being fed by the vessels, including for example, CO₂, proteins, polypeptides, antibodies, etc. Alternatively, the material may include a reservoir of material outside the vessel that is being released in a timed-control manner, thereby providing precise and long term dosing regimens by diffusion of material to the lumen and subsequent fluid flow (e.g., advection) that carries the material out of the bioreactor for collection, processing and/or purification.

“Cell population” refers to isolated and substantially purified cells that are introduced to a vessel network, such as a microvascular network, generated by any of the methods disclosed herein. Cell population may refer to a single homogeneous cell type, or alternatively, may comprise a plurality of distinct cell types. The plurality of cell types may be dispersed with one another, or may be restricted to particular spatial regions in the bioreactor. For example, endothelial cells may be seeded to physically contact the vessel wall, smooth muscle cells placed in a layer adjacent to the endothelial cells, and any number of cells of interest (such as tissue cells, cells indicative of a disease, e.g., cancer or tumor cells) that fill at least a portion of the remaining volume.

“Bioengineered cell” is used very broadly to encompass any means of altering the expression of one or more genes in a cell so as to produce a measurable, phenotypic change, such as overexpression of a gene product, or production of a gene product that is not normally produced, such as by genetic engineering.

“Gray scale illumination” refers to application of light in a pattern, wherein the pattern intensity can have a plurality of non-zero values. Such gray scale processes are particularly useful for generating more complex networks having variable heights within an individual polymer layer and for generating polymers with cross-linking densities that spatially vary. Materials having a lower cross-linking density can be preferentially removed by removal processes such as upon exposure to an etchant, as polymers with higher cross-linking density have slower etch rates and so are more resistant to the etchant. The typical black and white illumination pattern application, in contrast, generates a layer of uniform height. In addition, gray scale processes facilitate controlled permeability variation within a layer, and accordingly, provides the capability of tuning permeability along or within the network.

“Electrowetting” refers to application of an electric potential to a conducting fluid that substantially flattens the conducting fluid's surface, thereby substantially flattening an underlying photocurable liquid surface with a resultant decrease in surface dwell time. “Substantially flatten” refers to a surface that has at least 50% or preferably 75% or greater reduction in surface deformation at a particular point or averaged over the entire surface compared to a surface at the same time that is not undergoing the electrowetting procedure.

“Tissue volume” refers to the volume space external to the vessel network. For example, in an embodiment where the bioreactor is enclosed by walls, and the total volume of the bioreactor is V, then,

V=V_{L}+V+V_{TV}

Where V_{L} is volume of the network lumen, V_{w} is the volume of the network wall, and V_{TV} is the tissue volume. The three-dimensional nature of the manufactured networks and the ability to mimic in vivo microvascular network geometry provides a capability of maximizing V_{TV} and therefore the number of cells in the bioreactor, the amount of biological material collected and/or the implant size compared to conventional bioreactors.

The invention may be further understood by the following non-limiting examples. All references cited herein are hereby incorporated by reference to the extent not inconsistent with the disclosure herewith. Although the description herein contains many specificities, these should not be construed as limiting the scope of the invention but as merely providing illustrations of some of the presently preferred embodiments of the invention. For example, thus the scope of the invention should be determined by the appended claims and their equivalents, rather than by the examples given.

### Example 1

### Introduction to Microstereolithography

Stereolithography (SL) is a powerful fabrication technology which utilizes light-induced polymerization of liquid monomers to form complex three-dimensional shapes for a variety of purposes. Stereolithography is capable of fabricating complex three-dimensional solid structures using only light as a writing tool and liquid polymer as a base material. Stereolithography is a member of a larger class of photolithography technologies used for making silicon circuits and MEMS devices in the sense that all use light as a writing instrument.

The advent of microelectromechanical systems (MEMS) was brought about by the application of IC fabrication technology to the unique manufacturing challenges that exist at the microscale. The broad success of MEMS-based approaches has resulted in a multitude of new microsystems spanning a diverse range of applications from automobiles to biomedicine to counterterrorism. Despite the success of mass fabrication of highly uniform and integrated planar devices, silicon MEMS technologies show limitations for the fabrication of microdevices with truly 3-D complex geometries and largely depend on the use of bent or hinged structures. Additionally, standard silicon based MEMS devices require large and expensive cleanrooms for successful manufacture of components and are limited in the kinds of materials they may employ. The need for a versatile, less expensive, and fully 3D microfabrication technology was clear, and stereolithography was adapted to the microscale in response.

SL was originally conceived as a rapid prototyping technology. Rapid prototyping refers to a family of technologies that are used to create disposable true-scale models of production components directly from CAD in a rapid manner. “Rapid,” in this case, must be understood as “faster than before.” Individual parts may take hours or even days to fabricate, but these times represent vast improvements over the days to months the same components would have required by traditional means.

Such technologies greatly aid engineers in visualizing complex three dimensional part geometries, in detecting errors in prototype schematics, in testing critical components, and verifying theoretical designs at relatively low costs. Since most rapid prototypes can be made in a single day, physical 3D models can then be used to aid in the iterative design and testing of new components. Because dozens of prototypes can be made for the time and cost of a single prototype made by traditional methods, optimization of critical features and overall part quality can be greatly enhanced.

Early rapid prototyping success stories included the Defense Electronics Group of Texas Instruments, which used SL to enhance their product visualization capabilities, and Chrysler Corporation, which employed it to check dimensional tolerances of newly designed components [1]. Both companies reported savings in the tens of thousands of dollars on single development projects. At present SL boasts dozens of commercial users in the aerospace, agriculture, consumer products, electronics, and medical products industries [2].

Traditional SL: In a layer-by-layer fashion a traditional stereolithography machine will fabricate a complete part that accurately represents the original CAD drawings. First, a component's three-dimensional CAD drawings are “sliced” into a stack of 2D layers each of which posses a specified thickness. This thickness defines the vertical resolution of the finished prototype. Laser paths, often times described by simple G-Code, are generated for each 2D slice by the CAD software. (The CNC laser paths define the movement of a substrate mounted on an X-Y-Z stage; the laser itself is stationary.) Then a focused laser beam is rastered across the surface of a liquid monomer inducing polymerization in a local area around the laser beam spot. When a single layer is complete, the Z-stage drops the substrate beneath the liquid surface and the second layer is written. This process proceeds iteratively until all the layers are complete. When the component is finished, post-processing is required to remove excess liquid from the part. Specific polymer compositions may also require further curing.

Photocurable Monomer Resins: A monomer resin suitable for stereolithography applications is in fact a combination of at least three components: 1) a base monomer, 2) a photoinitiator, and 3) an absorber. The auxiliary components are important to the resin's performance. When photoinitiators absorb incident photons, they will form free radicals that provide the initiation and termination blocks for the polymer chain during polymerization. Absorbers are added simply to help the solution absorb more photons, as many of the stereolithography resins are nearly transparent. Together, the auxiliary components represent only a small fraction of the total resin weight.

A wide variety of esters, glycols, acryates, etc. can be used as base monomers provided the initiator and the absorber are selected to match the monomer's chemical properties. Widely used monomers for photolithography include hexanediol diacrylate (HDDA) and polyethylene glycol diacrylate (PEGDA).

Polymerization: The raw monomers are energetically stable at room temperature and require a catalyst in order to polymerize. When an initiator molecule becomes radicalized and interacts with a nearby monomer molecule, it will combine with the monomer in such a way that the monomer's structure is reorganized. The radical initiator bonds to one end of the monomer and the other end of the molecule chain is radicalized. The newly radicalized monomer is now ready to similarly bond with other monomers. This reaction continues until a radicalized monomer interacts with another radicalized initiator, which will then form a cap on the end of the polymer chain thereby halting the polymerization reaction. Polymer chains of this type can stretch hundreds or thousands of monomer units long.

This photopolymerization process is highly efficient; it requires 50-100 times less energy than thermal curing of similar monomers. Further, on average, for every two incident photons one radical will be produced. In common acrylic monomer solutions one radical would result in a polymer chain over 1000 units long. The net effect is that due to the ease of initiating a polymer reaction, stereolithography is able to employ UV lasers operating cheaply at relatively low power [1].

Successful management of the initiator molecules is important for the quality of the final part. Although polymerization rapidly slows when irradiation ceases, radicals do remain and can react with the polymer matrix for as long as several months, causing potential issues of stress concentration and warping [1]. Interaction with oxygen will return the initiator radicals to their stable state and prevent a polymerization reaction from occurring. For this reason SL is generally performed in an inert atmosphere.

The nominal exposure in the resin is given by the Beer-Lambert law of absorption:

$$E ( z ) = {E}_{o} exp ( \frac{- z}{{D}_{P}} ) \qquad ( 1 )$$

where: z is the depth into resin; Eₒ is the critical exposure required for polymerization; Dₚ is penetration depth of the monomer.

The penetration depth is a characteristic property of the monomer resin solution; it is heavily influenced by the absorber concentration. A process model to numerically simulate the curing behavior of the resin was described by Sun et al which also explored the effects of UV doping on the vertical resolution [4].

Microstereolithography (μSL) Transition to the Microscale: In addition to the benefits of prototyping capabilities in microsystems technology, μSL offers the potential for direct manufacturing of functional microdevices. μSL is attracting increased attention in this domain since its inception and continuous efforts are devoted in this direction in order to expand this technology to microfabrication applications. μSL has been used to build complex 3D microstructures as diverse as integrated microfluidic systems, photonic crystals, SMA actuators, etc. [8]-[10].

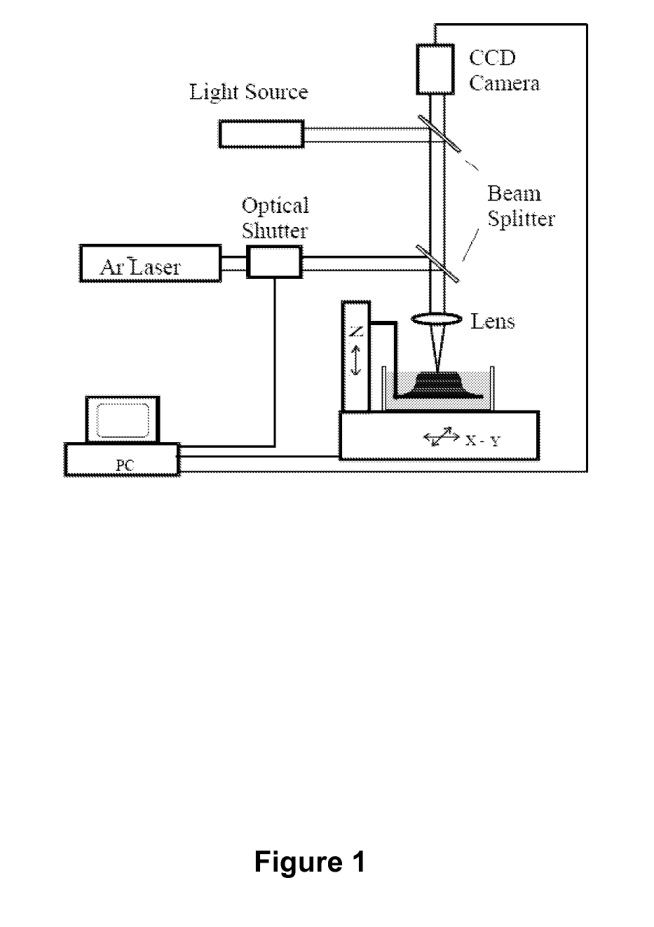

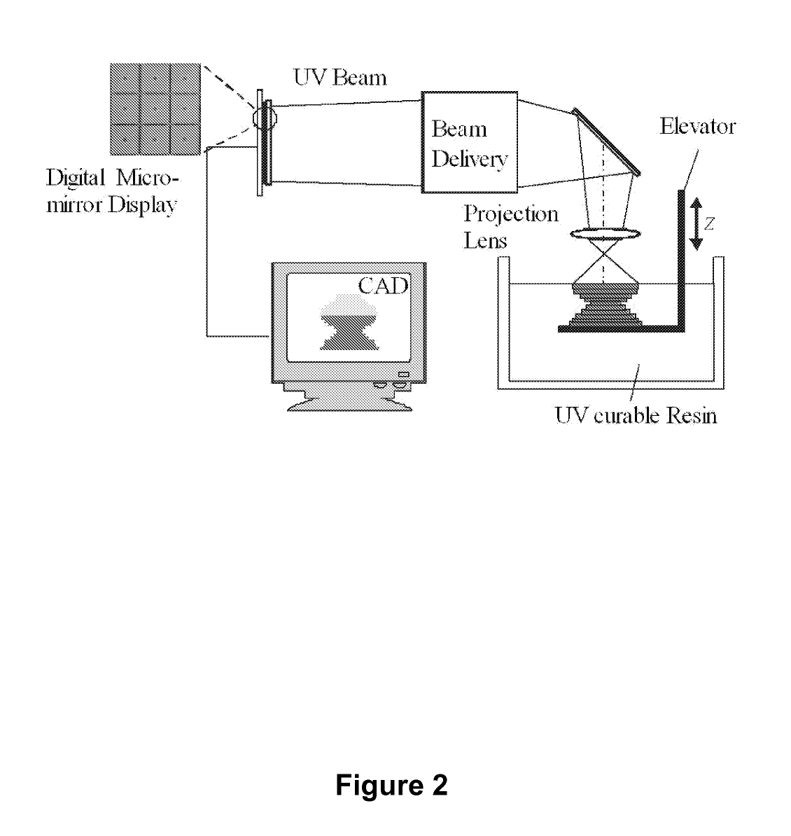

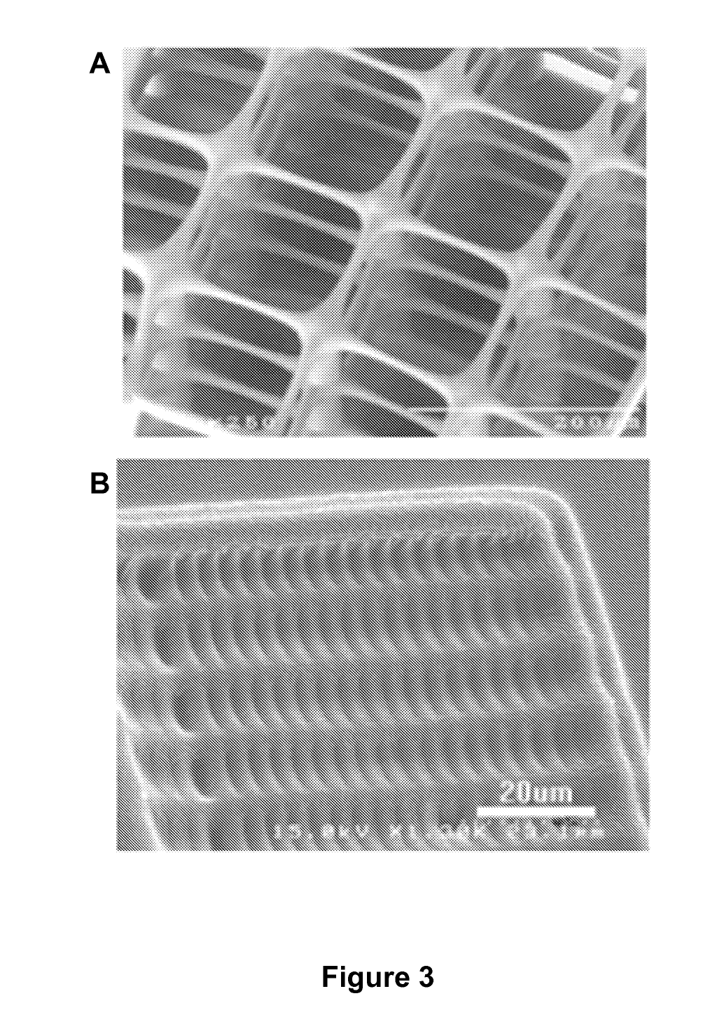

The traditional SL process described is a serial process, that is, it employs a laser with a discrete spot size to serially trace the path of the part to be created in a manner very similar to CNC milling of traditional materials. A serial μSL system is very similar to a traditional SL system except that the addition of optics allow for much smaller finished part sizes. FIG. 1 displays some of the necessary components of the standard μSL system.

Feature resolution is the critical parameter in developing high-quality microdevices. When initially developed by Ikuta et al. the standard μSL resolution (1H-Process) was 5 μm [11], which was roughly approximated by the area of the half-width of the laser beam spot used to scan the monomer surface. To further enhance the resolution of polymerized spot under the laser, Maruo and Ikuta developed both single-photon absorbed polymerization [12] and two-photon absorbed polymerization [13]-[15].

Single-photon absorbed polymerization utilizes a blue 441 nm He—Cd laser (100 mW) that focuses its beam inside the resin as opposed to on its surface. This was done for absorption purposes, but had the additional benefit of freeing the laser paths from 2D restrictions; fully 3D laser paths can be utilized with this method. The process takes place in an oxygen atmosphere so that oxygen molecules that diffuse from the atmosphere into the resin will scavenge the initiator radicals from all but the most-highly exposed areas of the monomer resin. Thus, only the most highly exposed areas in the center of the laser spot are polymerized. Furthermore, polymerization will begin only when the laser power is great enough to overcome the local concentration of oxygen molecules. The polymerization response of the resin is therefore highly nonlinear with respect to the incident laser power. Critical for the success of this process is the resin chemistry which must not strongly absorb the light at its blue frequency. This single photon method is capable of producing finished parts with lateral and vertical resolutions of 1.3 um and 2.9 um respectively [12].

In contrast two-photon absorbed polymerization relies on a completely different phenomenon to restrict the feature resolution. The two-photon approach similarly focuses the light underneath the surface of the liquid polymer. However, instead of polymerizing a UV-sensitive resin with a single UV-wavelength photon, the two-photon method requires two near-IR photons to be absorbed by the initiator at the same time in order to initiate the polymerization reaction. This is not a simple task. The photons must be generated by a high-powered Ti:Sapphire pulse laser to achieve the required photon energy and spatial density to initiate the reaction. The rate of two-photon absorption is proportional to the square of the light intensity, thus the threshold rate for polymerization is confined to a highly localized area in the center of the laser beam spot. Using this method the highest known resolutions for parts made by μSL have been created; Kawata fabricated a set of microbulls with a sub-diffraction limit resolution of 120 nm [16]. The major drawback of this two-photon approach is the expensive power requirements demanded by the UV resins that are relatively insensitive to IR light. Wang et al. have shown that it is possible to use lower-powered lasers when specially designed photoinitiators are employed [17].

The significant downside of the serial approach to μSL is the long process time required to raster across an entire surface with a small writing tool. For a constant area the production time increases with every improvement in the resolution of the beam. Projection μSL was developed to shorten these long process times.

Projection μSL: Projection SL is also a layer-by-layer process that reproduces the original CAD drawings in a 3D model. However, instead of generating laser paths from the 2D CAD slices, the slices themselves in the form of digital information (e.g., .bmp images) are used as masks. UV light from a flood UV source is reflected off a dynamic mask generator that displays the bitmap images (both liquid crystal displays and digital micromirrors have been used as containing the bitmap image) and is optically routed by means of mirrors through a projection lens which reduces the image to the desired size. The image is focused on the surface of the monomer resin exposing the entire layer simultaneously. Thus, the time required to process an individual layer is dramatically shortened and rendered independent of the image geometry. When a layer is fully exposed, the Z-stage drops the substrate beneath the liquid surface, the dynamic mask generator displays the next image, and the next layer is exposed. This process proceeds iteratively until all the layers are complete. In comparison to serial systems the resolution of projection systems is relatively poor because the flood UV source cannot be localized. Bertsch, limited by the pixel resolution of the dynamic mask generator, reported a resolution of 15 μm [18] for the fabrication of a set of helical cogs. Sun et al., by employing a much higher resolution digital micromirror, achieved resolutions of 600 nm by using a UV light source at 365 nm for high aspect ratio wire matrices and helixes fabricated in HDDA [4].

Because projection stereolithography (PSL) requires the use of lens and mirrors, the optical quality of the system is an important feature governing the overall part quality. The relationship between the lateral and vertical resolutions of a component with the optical quality of the PμSL system and the photosensitivity of the resin using the modulation transfer function (MTF) of the optical components is described. In general, as the modulation of the optical system degrades with higher spatial frequency, it is increasingly difficult to reproduce fine features with short spatial periods during one exposure than those with large spacing [19]. Proper understanding of the modulation limits of projection optics is important for correct design of microcomponents manufactured with PμSL systems. As dynamic mask generator resolution increases advances in projection stereolithography resolution are still possible, however, given the flood nature of the process, the diffraction limit for a given wavelength is not likely to be surpassed.

Throughput: Layer-by-layer fabrication and dedicated light source requirements tend to limit the throughput of microstereolithography systems. Various means are proposed to decrease layer generation time. Ikuta has developed a serial, laser-based “Mass IH” process that uses arrays of optical fibers to solidify identical microfluidic devices (˜50) simultaneously on a silicon wafer [20]. The wafer is then etched to release the separate devices. This method is elegant for producing moderate numbers of complicated structures, yet shear stress issues involved in the motion of optical fibers have not been addressed. Such stresses hinder the ability to generate fragile structures.

Alternatively, a continuous-flow approach using a projection microstereolithography setup that polymerizes simply-shaped components in a flowing stream of polyethylene glycol diacrylate (PEG) provides high throughput [21]. The PEG flows in a PDMS microchannel that is 20 μm deep (the channel composition is critical to the fabrication success due to the oxygen transfer properties of the PDMS). Throughputs of 400,000 components per hour were reported. Various solids (rectangular, triangular, and hexagonal prisms) roughly 45 μm square by 15 μm thick were successfully fabricated. Smaller feature sizes are limited only by the optical power of the system. This method provides for the fabrication of bi-material components by flowing streams of immiscible polymers together in the microchannel. This method is effective for producing large numbers of identical components that may be used as building blocks for self-assembled structures.

Mass production for parts with complex, multi-layer geometries requires a significant reduction of the single layer fabrication time. The layer processing time relationship is given in Equation 2:

Single layer fabrication time=exposure time+surface dwell time (2)

Because the exposure time for a given layer is fixed by the monomer chemistry and the thickness, it cannot be reduced by process improvements. After the exposure of each layer, the Z-stage lowers the sample beneath the liquid surface of the monomer such that it will flow over the top of the existing structure. The monomer resin takes time to completely recoat the previous layer and thoroughly settle before the next layer is exposed or dimensional accuracy is compromised. The elapsed time between layer exposures is known as the surface dwell time and can consume up to 65-90% of the single layer fabrication time.

For viscous fluids the dwell time between layer exposures can be minutes. As most of the biocompatible/degradable polymers are quite viscous, and given the fact that biomedicine is likely to continue to play a significant role in the development of microstereolithography, reducing the viscous force effects facilitates mass production of microcomponents.

With a wide array of potential applications and available submicron feature resolution, μSL is positioned to be a popular fabrication technology for years to come. Its inherent advantages are numerous [8] and include: True three dimensional structures with no sacrificial supports required; High aspect ratios (>10); Uses dynamic, computer-generated masks; Simple low-cost apparatus; Minimal equipment footprint; Low safety risks; Low power light sources; Low material waste.

Microstereolithography is one of several microfabrication processes currently being used to manufacture components on the microscale. Table 1 is a comparison summary of μSL with other popular technologies.

The reliance of SL on photopolymerization has traditionally restricted the kinds of materials that may be used to form μSL parts. However, recent work in polymer chemistry has opened doors for new materials and promises to greatly expand its range of applications. Besides the conventional acrylate or epoxy resins that are used as structural materials, soluble polymer has been introduced as a sacrificial material. Apart from the conductive polymers that can be directly patterned using μSL [8], metals are incorporated by electro-deposition [2] or laser plating [22]. Furthermore, other functional materials such as PZT ceramic are also successfully introduced in the process via direct writing or transfer molding [10].

The invention of ceramic-impregnated photocurable polymers has led to numerous innovative polymer-ceramic microstructures. These resins contain 50%-80% alumna nanoparticles by weight suspended in a traditional low-viscosity acrylate polymer. The inclusion of metal particles within the polymer matrix allows for the fabrication of electrically conductive components. After the polymer is hardened, the green components require a post-process firing to debind and sinter the alumna particles into a ceramic part [10], [23]. Provin, in similar fashion, has fabricated polymer-ceramic microcatheter terminals for medical use, microgimbals for use in robotics, and microchannels for bio-analysis [24]. Although these results demonstrate the significant potential from these resins, further material challenges must be overcome to improve the strength of these materials.

Biomedical Applications: The key research area currently driving the development of μSL is biomedicine. The recent explosion in biotechnology has increased the demand for microfabrication with bio-friendly materials. Microsterolithography's ability to fabricate small repeatable structures with biocompatible polymers makes it an ideal choice for many biomedical devices. Current biomedical research areas include tissue scaffolds, drug delivery, and modeling of biological systems.

Tissue Scaffolds: Substantial effort is being made to fabricate tissue engineering scaffolds which will support live tissue growth for organ transplants, reconstructive applications, and research into cell behavior [25]. Microstereolithography is one of the technologies on the forefront of this effort.

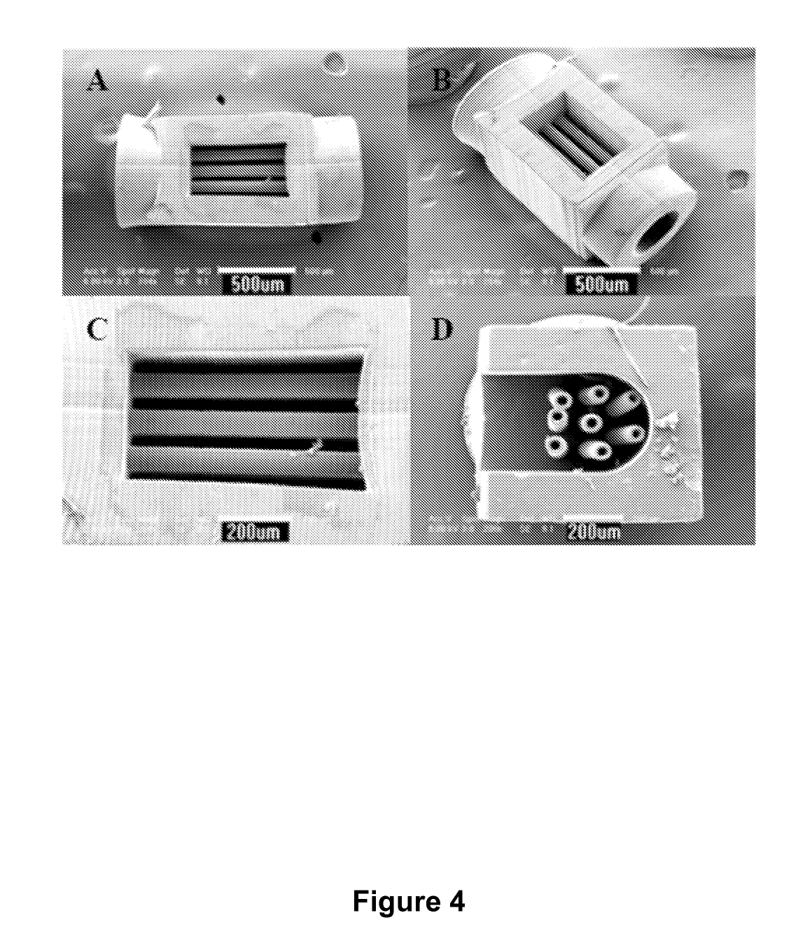

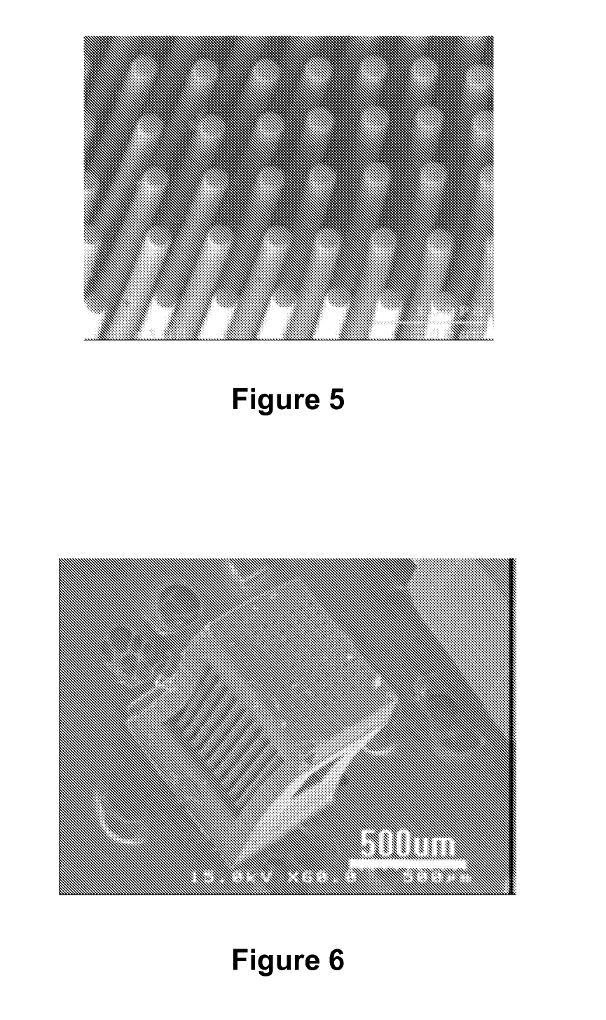

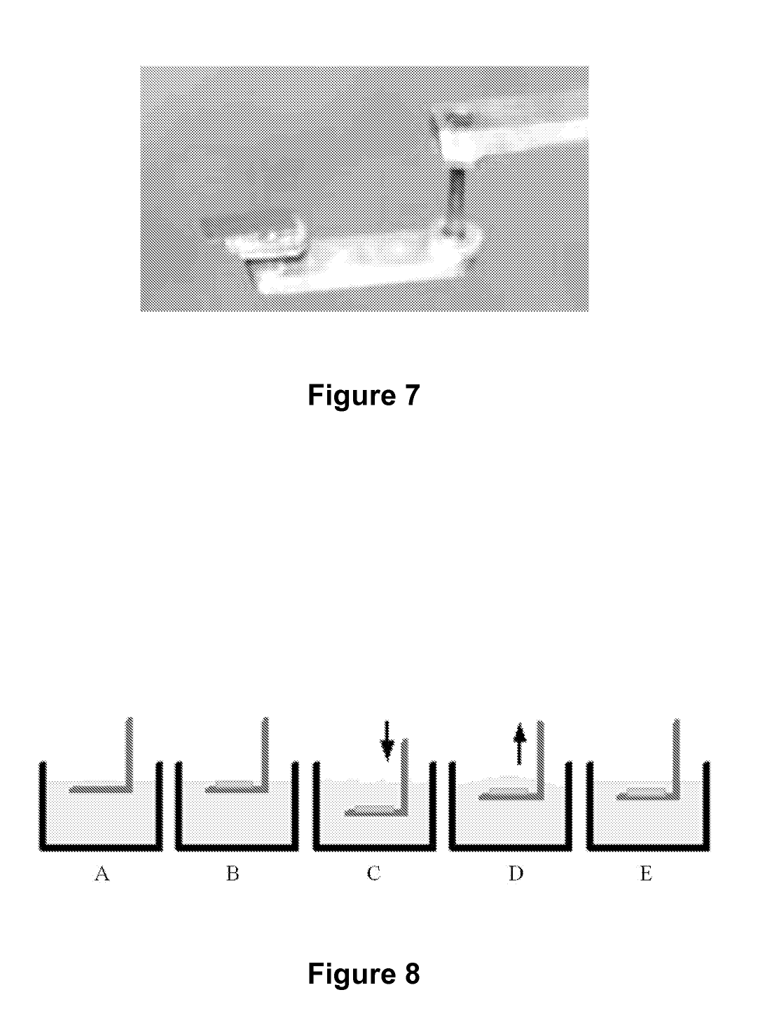

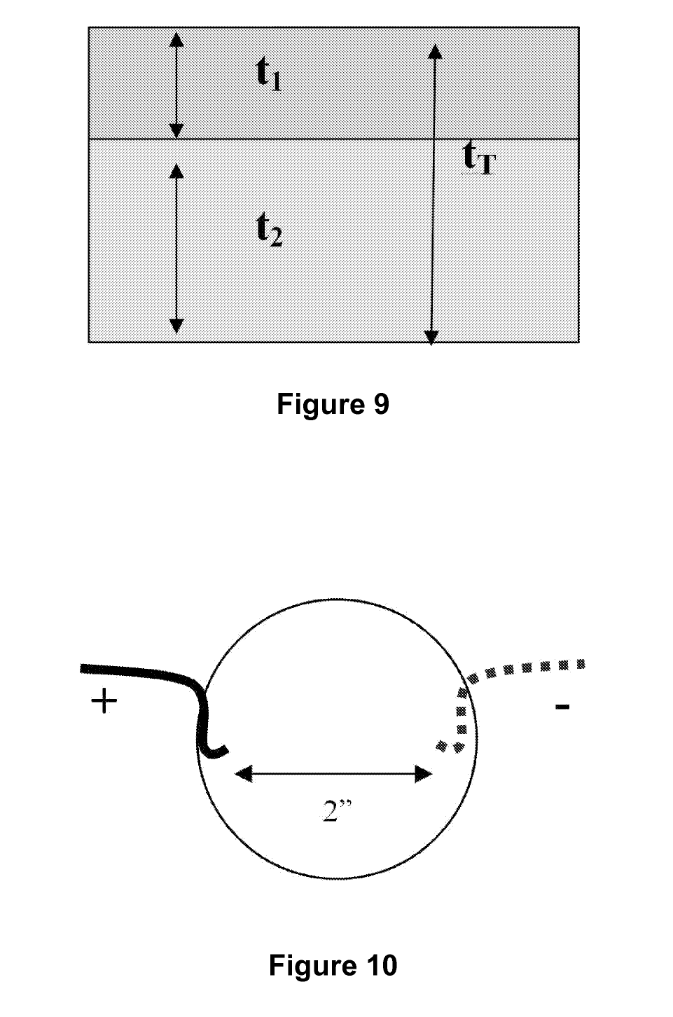

Xia et al utilized PμSL to fabricate a microbioreactor made from polyethylene glycol that provides active transport of nutrients and oxygen within a cell culture matrix [26]. These bioreactors provide a means to artificially control the local environment of the culture media. A network of such bioreactors, as shown in FIG. 4, connected by microtubules shown in FIG. 5 is used to grow cell cultures that are significantly thicker than is currently possible using diffusion-only based approaches, leading to advances in tissue engineering.

Lu et al have fabricated polymer tissue scaffolds with unit cells of several hundred square microns [27]. The working polymer was enhanced with controlled-release bio-factors before polymerization to promote cell growth. By altering the composition of these bio-factors for each new layer of scaffold, locally unique microenvironments were created. The finished scaffolds were then used as test chambers to study the differentiation of osteogenic stem cells.

Drug Delivery & Detection: Critical to the success of time-released in vivo drug delivery is the minimization of the body's immune reaction to the presence of a foreign body. Microstereolithography's ability to impregnate compounds within its polymer structure makes it an excellent choice for embedded drug solutions. Due to the remarkable surface to volume ratio, a hollow polymer micromatrix could be advantageously employed as capsules for fast DNA or protein sensitive drug release. For the direct integration of functional polymers with molecular recognition into the micromatrix, Conrad et al fabricated functional, three-dimensional, molecularly imprinted microstructures with recognition for small segments of DNA and its derivatives [28]. Preliminary result on the external flow through microfabricated 3D matrix already demonstrated a significant improved binding rate under fluorescent imaging. Given that a wide range of target analytes are responsive to this molecular imprinting technique, the microfabricated polymer devices could serve as smart carriers for a wide variety of drugs, pesticides, peptides, and proteins. Kwon and Matsuda tested a series of cone-shaped, μSL-fabricated, PEG-based polymer structures in rats for durations of 1-4 weeks [29]. They observed surface erosion of the structures made from low weight PEG (MW200). They also observed surface and bulk erosion of the structures made from high weight PEG (MW1000). The drug-loaded heavy PEG also demonstrated the least amount of inflammatory response from the host's immune system. These kinds of micro-needle structures show promise for use in localized in vivo applications.

Modeling of Biological Systems: Ikuta and Maruo have developed a series of biochemical integrated circuits using μSL. These microreactors have a wide range of applications including medical sample testing, protein synthesis, and biochemical computing. Their operation requires the presence of micro-electrostatic actuators which were also patterned by μSL and subsequently electroplated. These biochemical IC's may be used in large arrays to simulate biological systems [11], [30]-[32]. Stereolithography has also been used on the mesoscale to aid cardiologists and oncologists with the implantation of medical devices. Binder et al have reported that polymer models of patients' cardiac networks have been fabricated. They will likely be used to aid doctors in planning for surgeries to correct damaged valves and congenital heart conditions [33]. Poulsen et al have reported the use of SL to create a model of a patients' brain tumor and surrounding tissue from CT scans [34]. The model was used as an aid in planning the surgery, leading to a successful tumor treatment. Similar applications of μSL to biological modeling of specific patient needs are being developed.

Projection Microstereolithography System: Here brief descriptions are given of the projection microstereolithography (PμSL) system used to fabricate a variety of 3D micronetworks. The PμSL system sits easily on a 4′×4′ vibration damping table.

Control System: The PμSL system is controlled via a Labview interface on a standard Windows PC running Windows XP. It governs all of the real-time fabrication steps required to position the substrate (X & Z) and turn the UV source shutter on and off. In addition it includes manual controls for starting and stopping the fabrication process and a series of process input settings. The interface is custom designed.

Translation Stages: Three Newport Viper V translation stages (two vertical and one horizontal) position the substrate holder. They have a speed of 1000 μm/s and a range of motion of roughly 10 cm. They are driven by a Newport MM 3000 Motion Controller which is in turn operated by the Labview control program. They position the substrate during all focusing and fabrication sequences.

Substrate Holder: The substrate holder (FIG. 7) is a U-shaped aluminum support for a small rectangle of silicon. It allows the substrate to be properly positioned in the liquid polymer (FIG. 8) and aids in easy removal of the finished sample from the resin. The substrate provides a surface for the polymerizing resins to adhere to and is important for successful fabrication.

UV Source: The projection microstereolithography system's light source is an Oriel 87435-1000-1 mercury lamp that projects high-intensity (200-500 W) light at a wavelength of 435 nm. It is powered by an Oriel 68810 arc lamp power supply and tuned by an Oriel 68850 Light Intensity Controller. Light leaving the source travels downward into a double prism which reflects the light onto the LCD projector's chip.

LCD Projector Chip: The LCD projector displays the sequential bitmap images on its LCD chip. Each image masks the incoming UV light to create the pattern for the layer currently under exposure. The chip pixel area defines the possible exposure area, and, at present, this is the most significant limit to the maximum rectangular area of the finished component. The trade-off between feature resolution and maximum area is a fundamental restriction of the discrete pixel size. When reduced through the minimizing lens each image pixel corresponds to a square 1.1 μm in length resulting in a maximum total area of 1200 μm. In this case the LCD chip was removed from the LCD projector housing for easier exposure. The projector's built-in lamp is not utilized.

The light reflected off the LCD chip travels back through the double prism, through the beam splitter, and is reflected off the single mirror to the lens below. The overall light path must place the LCD in the proper position for the image to be tightly focused on the surface of the liquid polymer, based on the focal length of the lens. These distances are calculated from the Gaussian lens relation:

$$\frac{1}{o} + \frac{1}{i} = \frac{1}{f} \qquad ( 3 )$$

where o and i are the object and images distances from the lens, respectively, and f is the focal length of the lens. Note: these distances as calculated above would not be sufficiently accurate to produce acceptable part resolution; the system requires a dynamic control system to properly control the focus of the incident image.

Minimizing Lens: The minimizing Zeiss lens reduces the size of the incoming image by a factor of ten and projects the image onto the liquid surface. The lens also allows light reflected from the substrate to return to the CCD camera positioned above the beam splitter.

CCD Camera: The CCD camera, positioned above the beam splitter, receives light from the sample surface that returns through the system optics. This image is displayed by the Labview interface and is used to focus the system prior to fabrication.

Nitrogen Atmosphere: The liquid polymer sits inside of a clear Plexiglas box during fabrication. The front face is removable to allow for monomer access. Nitrogen is pumped into the box from standard N₂ tanks nearby, thus displacing the oxygen present in the standard atmosphere. A heavy nitrogen atmosphere is important for successful polymerization of the base monomer as it prevents atmospheric oxygen from scavenging the initiator radicals. The box has bottom access for the Z-stage and side access for the substrate holder.

Layer Slicing: The bitmap image slices are created using a custom macro written for AutoCAD. Any three dimensional AutoCAD object may be sliced using this program. CAD models from other modeling packages may be used as long as models can be converted into AutoCAD. Briefly, the macro records the cross section of the 3D solid on a moving datum plane and saves this black and white snapshot as a bitmap image. The datum plane locations are specified by the user-supplied layer thickness. The bitmap images are stored in a user-defined folder.

Microstereolithography System Operation: Focusing: When the substrate enters the Plexiglas box its vertical stage will traverse downwards until the CCD camera captures a highly focused image of the silicon surface. The liquid monomer will then be raised to completely cover the substrate. The monomer's stage will then slowly transverse downwards until a highly focused (though much dimmer) image of the silicon substrate is visible in the CCD camera. The focusing criteria rely on the contrast ratio of pixels near the substrate image boundary. By recording these two focused positions, the Labview control program has sufficient information to position the stage for each successive layer, thereby giving a uniform thickness to the layers. The entire focusing process takes about five minutes, though this will vary somewhat based on the opacity and viscosity of the monomer resin.

Fabrication: When the substrate is in the focused position the substrate rests below the liquid monomer surface at a depth of one layer thickness (FIG. 8A). After exposure the monomer above the substrate will harden into solid polymer (FIG. 8B). The substrate holder stage will transverse downward to allow the liquid monomer to flow over the substrate (FIG. 8C). The stage will come to rest at a position one layer thickness beneath its previous position (FIG. 8D). Here, the fabrication process must wait for the monomer surface to flatten, otherwise the resolution and dimensional accuracy of the finished part will be compromised. When the surface is level (FIG. 8E) the next layer may be patterned. This process repeats until all layers have been patterned. Samples 1.1 mm² in area and 3 mm tall (300 layers) have been made using this projection microstereolithography system. Higher area LCD chips, or using a plurality of LCD chips provides access to larger-area layer fabrication. In addition, taller patterns may be produced by increasing the number of layers patterned.

Sample Removal: Removal of the sample from the substrate must be done carefully to ensure that the sample is not damaged. All samples are removed from the silicon substrate and/or unpolymerized polymer by means of vertical suspension in an ethanol bath; the ethanol breaks the bonds between the solidified polymer and the substrate, and the sample will slide off the substrate into the ethanol. Forced removal of the sample from the substrate may result in damage to the sample; however robust structures may be removed from the substrate by tweezers. Delicate components may be super-critically dried in CO₂ to prevent the meniscus forces of the evaporating ethanol from destroying the weak structures.

Microstereolithography has demonstrated its capability to fabricate a wide variety of microstructures and devices across a broad range of engineering disciplines. Its two main configurations, serial and projection, each have their strengths and weakness but projection microstereolithography shows the greater promise for commercialization at the microscale due to its decreased fabrication times.

### Example 2

### Enhanced Microstereolithography Via Electrowetting-Induced Surface Flattening of a Two Fluid Interface

One of the major hindrances to the progress and eventual commercialization of microstereolithography technology is the long dwell times required for viscous polymers between fabrication of successive layers; the viscous forces of the heavier polymers resist the flattening efforts of gravity.

One potential solution for this problem involves constraining the surface of the liquid using a transparent plate. The plate, if positioned slightly below the liquid surface, would ensure a flat liquid surface and a uniform layer thickness without the long dwell times required by the free surface approach. However, as described by Ikuta, there are serious obstacles to this approach due to secondary polymerization of the resin on the underside of the plate [35]. In short order secondary polymerization on the plate's surface renders it too opaque to allow the light to pass through it. Additionally, the polymer build up on the underside of the plate could physically damage the desired structures forming on the substrate.

It has been demonstrated that by coating the plate with a low energy Cytop surface the fabrication time could be successfully reduced 65-90% without secondary polymer buildup. However, when the plate contacts the liquid surface, pressure-driven flow is induced around the sample. If the sample is delicate and especially if it contains high-aspect structures the sample can be destroyed by the moving fluid. In summary, all of the constrained surface PμSL approaches have significant limitations; full commercialization of the technology will be difficult until the dwell times can be reduced. Therefore, electrowetting is hereby proposed as a mechanism to reduce the layer dwell time.

Wetting Characteristics of Fluids: The wetting characteristics of fluids have long been characterized by the Young-Dupré equation:

$$Cos θ = \frac{{γ}_{SV} - {γ}_{SL}}{{γ}_{LV}} \qquad ( 4 )$$

The relation describes the balance of the interfacial energies on the solid-liquid, solid-vapor, and liquid-vapor boundaries. In this case where the vapor (or atmosphere) is relatively thin, the solid-vapor and liquid-vapor energies may be regarded as the surface energies (tensions) of the solid and liquid, respectively. The relative strengths of the solid and liquid energies are critical, not their absolute values.

However, it is very difficult to measure the interfacial solid liquid energy on a given surface, thus the contact angle of the fluid is measured instead. The contact angle therefore serves as a parameter for characterizing the relative strength of a liquid's hydrophobicity or hydrophilicity, provided an identical solid surface is used to test the fluids being compared.

Electrowetting: “Electrowetting” is a technique that uses an applied electric field to alter the surface morphology of a liquid interface. The electric field can alter both the shape of single droplets and the orientation of a bi-fluid interface, thereby modifying the contact angle in both cases. A highly useful effect with broad applications, the effect has recently been used to create and actuate miniature lenses. Chen et al created a variable lens using a single droplet, while Kuiper at al have demonstrated a two fluid lens system [36]-[37]. Electrowetting is a difficult phenomenon to describe reliably due to its high dependence on local surface imperfections. However, Lienemann at al have attempted the simulation and optimization of electrowetting-induced droplet splitting [38]. Nevertheless, electrowetting's usefulness has been demonstrated empirically.

Kuiper and Hendricks used the meniscus formed between two immiscible fluids as the optical lens for a miniature achromatic camera (one fluid is conductive, and the other is not). They demonstrated real-time tuning of this lens by applying an electric field between two specially designed electrodes placed perpendicular to the fluid interface. The applied electric field effectively reduces the interfacial tension between the two fluids and the meniscus boundary between them shifts to accommodate the new effective force balance. By adjusting the voltage the curvature of the lens can increase, decrease, or even invert, thereby altering its focal length and permitting dynamic focusing.

The curvature of the lens is quantified with the contact angle that the fluid interface creates with the electrode side wall. The contact angle is given by a modified form of the Young-Dupré equation, which accounts for the effect of the applied voltage:

$$cos θ = \frac{{γ}_{wi} - {γ}_{wc}}{{γ}_{ic}} + \frac{ɛ}{2 {γ}_{ic} {d}_{f}} {V}^{2} \qquad ( 5 )$$

Where:
∈ is the dielectric constant of the insulating film,
d_{f} is the thickness of the insulating film
V the applied voltage
γ_{ci} the liquid/liquid interfacial tension
γ_{wc} the interfacial tension between the wall and the conducting liquid
γ_{wi} the interfacial tension between the wall and the insulating fluid

If a flat surface is desired, then the equation may be rearranged to solve for the voltage required to produce a flat voltage for a given fluid system. Taking cos(θ)=0 and solving for V, the voltage required to induce a flat surface is given by:

$$V = \sqrt{\frac{2 {d}_{f} ( {γ}_{wc} - {γ}_{wi} )}{ɛ}} \qquad ( 6 )$$

This equation predicts that the flattening voltage is not dependent on the diameter of the container housing the resin.

Electrowetting Electrodes: Electrode Design: Practical electrowetting can be achieved only by employing specially designed electrodes. As seen in Equation (5) the contact angle modification provided by electrowetting depends on two significant factors: the strength of the electric potential, V, and the thickness of the dielectric layer, d_{f}, covering the surface of the electrodes. The strength of the electric field can be easily modified with any variable voltage source, but the absolute value of the applied voltage should be kept as low as possible to enable the use of small, inexpensive voltage sources, and to reduce any possible safety hazards. The dielectric layer itself must be tuned to meet two competing conditions: it must be thick enough to resist dielectric breakdown, but it must be thin enough to allow substantial contact angle modification at moderately low voltages (0-150V). Thus, the dielectric material and thickness are the critical design criteria for a successful electrowetting system.

Electrode Fabrication: The base of the electrode is a single-sided polished {1 0 0} silicon wafer. Silicon is rigid and durable and allows for easy cleanroom processing. A thin evaporated chromium layer is evaporated onto the silicon wafer using an electron beam evaporator to promote adhesion between the silicon and the nickel layer. Evaporated nickel is deposited over the chromium to form the electrode; nickel is selected for its excellent solderability. Polyimide, with its high dielectric constant, is spuncoat on top of the polyimide to form the insulating layer. Two layers of Teflon coat the polyimide for physical protection, to increase the hydrophobicity of the surface, and to further enhance the dielectric constant. Thus, in this case the dielectric layer is actually a composite layer of both polyimide and Teflon. Their respective layer thicknesses are measured with a KLA Tencor Alpha Step-IQ Surface Profilometer and are given in Table 2.

The composite dielectric constant of a bi-layer thin film system shown in FIG. 15 is given in Equation (7):

$${κ}_{T} = \frac{{κ}_{1} {κ}_{2} {t}_{T}}{{κ}_{1} {t}_{2} + {κ}_{2} {t}_{1}} \qquad ( 7 )$$

∈=κ_{T}∈ₒ (8)

Equation (8) then gives the permittivity of the bi-layer system. The calculated composite permittivity of the Teflon-Polyimide system for the given thicknesses and dielectric constants is 3.07e⁻¹¹ F/m.

Characterization of System Fluids: System Fluid Conductivity: Since the ultimate goal of the effort is to fabricate components via stereolithography, a widely used biocompatible stereolithography resin, polyethylene glycol diacrylate, is chosen for the conducting fluid. Standard polyethylene glycol diacrylate (PEGDA) is mixed with an organic salt, Imidazole Trifluoromethanesulfonate 5% by weight, to allow it to conduct electricity. Octane was chosen as a convenient non-conductive fluid. Numerous solution variants are achieved by employing PEGDA solutions of differing molecular weights: 258, 575, & 6000.

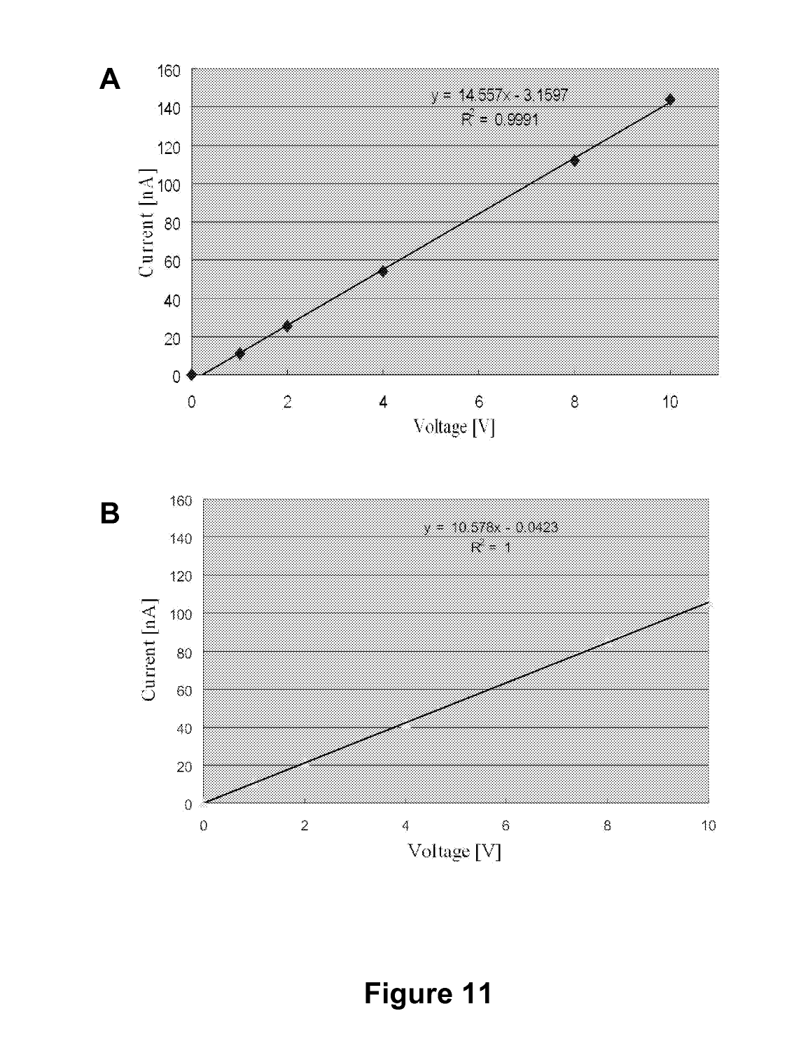

In order to establish this system's conductive properties, current was passed through a solution of PEGDA placed in a standard Petri dish via two electrical wires placed two inches apart (FIG. 10). The voltage output and the current measurement were controlled by a Keithly 6487 Picoammeter. FIG. 11A shows the control case of pure PEGDA-258. Here PEGDA-258 acts as a linear resister of over 70GΩ, allowing only 140 nanoamperes of current flow at 10V.

Sodium chloride, 5% by weight, was added to the PEGDA-258 resin; the solution was stirred for 3 hours at 30° C. and the experiment was repeated. FIG. 11B shows that the salt did not dissolve in the monomer solution and did not enhance its conduction. The salt crystals were still visible after stirring. This result was expected but demonstrates the need for an organic salt.

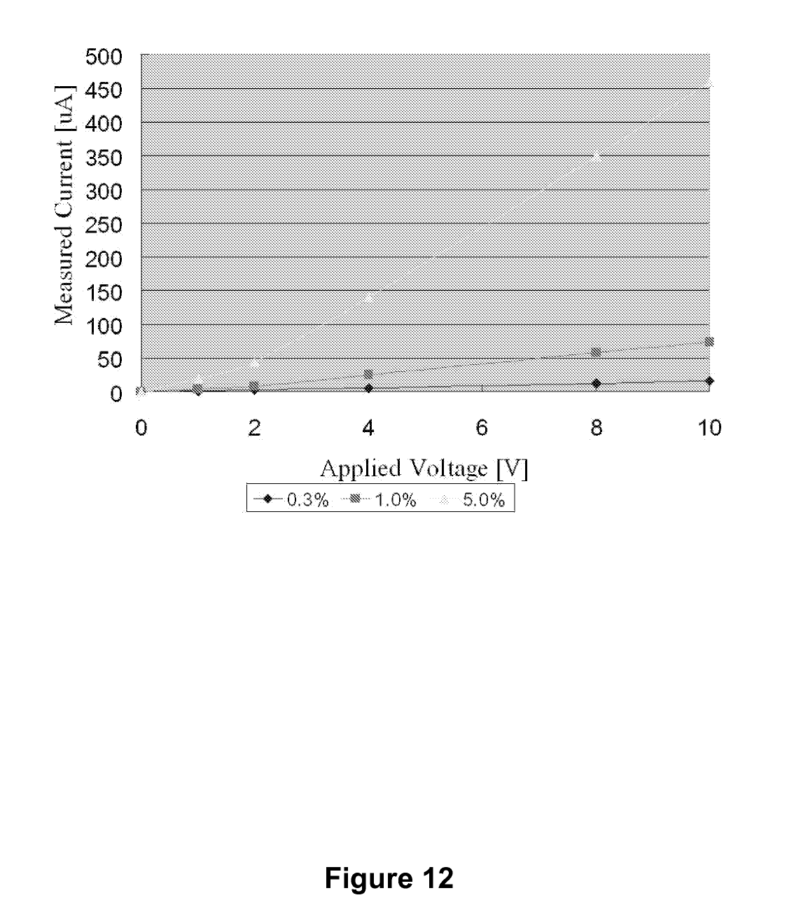

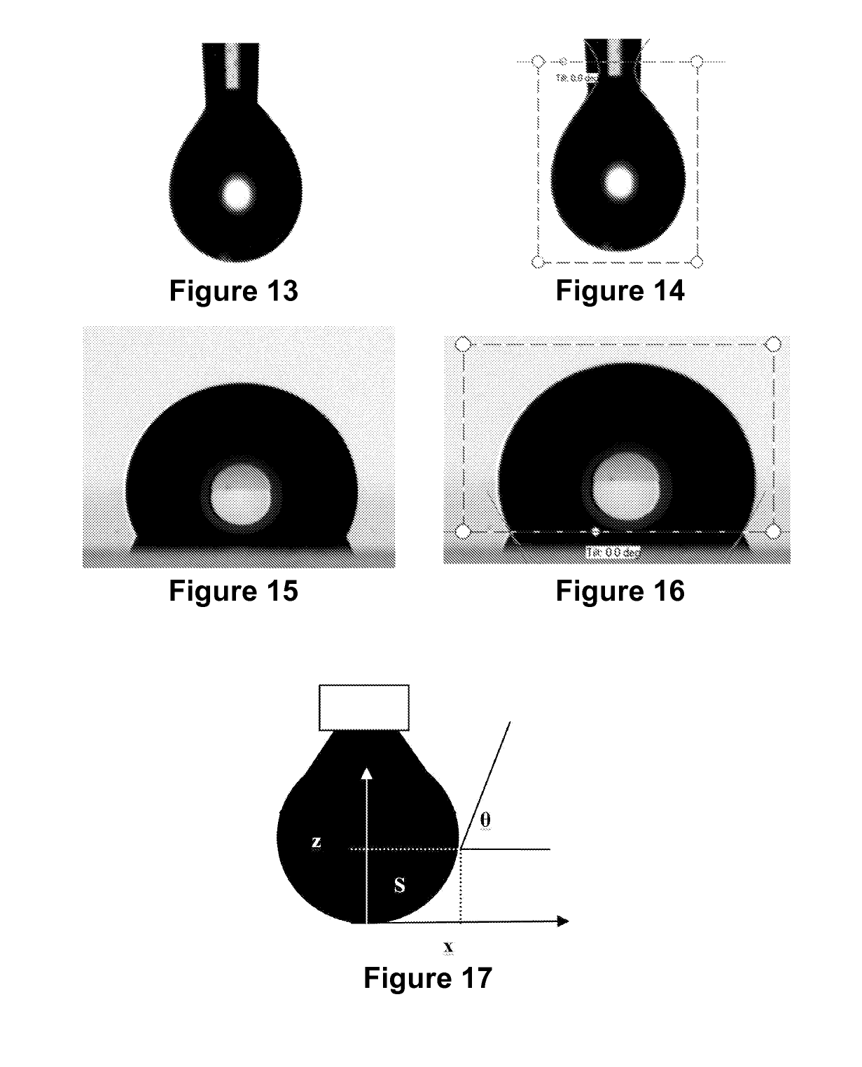

Imidazole trifluoromethanesulfonate salt (ITFMS), at 0.3%, 1.0%, and 5% by weight, was added to the PEGDA-258 resin; the solutions were stirred for eight minutes at 30° C. FIG. 12 shows the dramatic increase in conduction permitted by the dissolved organic salt. The 5% solution at 10V conducts 450 μA, an increase of 3200 times over the unsalted PEGDA-258.

Table 3 shows that the 5.0% salted PEGDA-258 (SPEGDA-258) has a resistivity on the order of ordinary tap water; therefore SPEGDA-258 may be considered as a conducting solution. Using the same setup, the resistance of Octane at 200V was found to be 1.54×10¹² Ohms thereby demonstrating its suitability as a non-conducting fluid.

System Resin Surface Energies: The surface energy of a liquid can be determined directly by an optical measurement of its surface tension. Via Equation 4, the contact angle of a liquid droplet and its surface tension is used to calculate the interfacial energies of a fluid system. These quantities are crucial for determining the required flattening voltage by Equation (5).

Fortunately, both parameters can be accurately measured using an optical instrument known as a goniometer. The goniometer consists of a blue light source on the far end, a stage in the center, and a camera on the near end. The camera is model DMK 21F04 from the Imaging Source. The images taken by the camera are processed by the CAM Optical Contact Angle and Pendant prop Surface Tension Software v. 3.74 running on a nearby desktop PC.

For both measurements a liquid drop is placed between the blue light and camera. The blue light is absorbed by the droplet resulting in a high contrast image of the surface that is captured by the camera. The contours of the droplet are then traced by a curve fitting algorithm. The interfacial energy of a pendant droplet forming the boundary between any two fluids can be found by:

$$γ = \frac{Δ ρ g {R}_{o}}{β} \qquad ( 9 )$$

Where: γ=interfacial tension; Δp=difference in density between fluids at interface; g=acceleration of gravity; Rₒ=radius of drop curvature at apex; 6, the shape factor of the droplet, is calculated computationally by solving the system of equations:

$$\begin{matrix}\frac{d x}{d s} = cos ( θ ) & ( 10 ) \\ \frac{d z}{d s} = sin ( θ ) & ( 11 ) \\ \frac{d θ}{d s} = 2 + β z - \frac{sin θ}{x} & ( 12 )\end{matrix}$$

The goniometer software measures Rₒ, z, and x from the curve trace, as shown in FIG. 17, and calculates β. In this case, where a single liquid is suspended in air, the calculated interfacial energy is assumed to be the same as the surface energy of the liquid, as the surface energy of air is extremely low. For this case Δp is input by the user as the density of the pendant liquid.

For a contact angle measurement, the traced curve's outline is regressively fitted to the Young-Laplace equation. The derivative of this equation, represented by the tangent lines in FIG. 16, is taken at the point of intersection between the droplet and the surface. The contact angle is determined by taking the arc tangent of the derivative at the intersection point. The goniometer's software performs the necessary calculations.

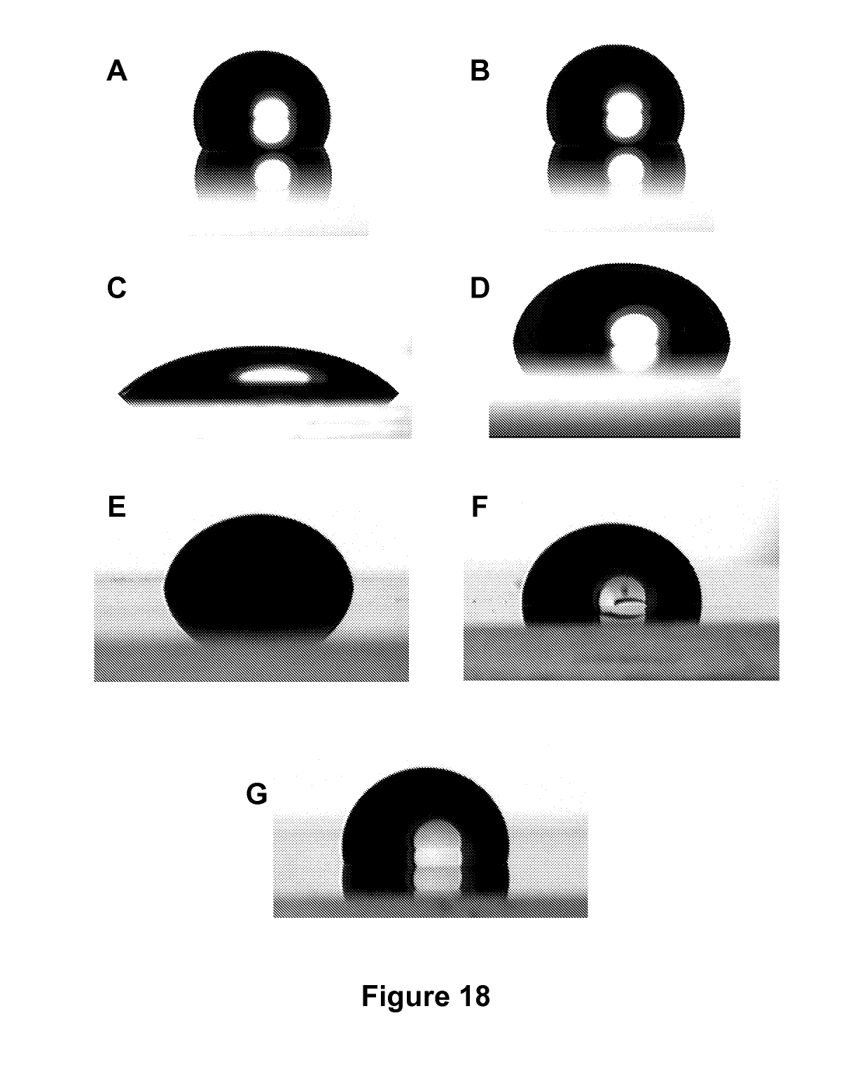

Results of Surface Tension and Contact Angle Measurements: All of the system resins' surface tensions and contact angles were measured as described above. The contact angles were measured on two different Teflon AF 1600 surfaces. The first Teflon surface was spuncoat over a microscope slide and then cured. The second was dipcoated and cured. Contact angles for system resins on both surfaces are given in Tables 4-5. FIG. 18A-G display single droplet profile views (as seen by the goniometer) of the monomer resins under investigation.

A comparison of the results in Tables 4-5 support several conclusions: (1) the two surfaces, dipcoated and spuncoat Teflon do not vary significantly in their average surface energies; (2) the dipcoated Teflon surface displays much less variance around the mean value and is therefore judged to give more reliable data; (3) the presence of Irgacure photoinitiator and Sudan absorber in the SPEGDA-258A solution does not significantly change SPEGDA-258's liquid-solid interaction with the Teflon surface; (4) Increasing the molecular weight of SPEGDA increases the contact angle of the salted monomer.

Measured surface energies and published densities of the monomers are given in Table 6. Published surface energies of similar Sartomer monomers are given in Table 7.

A comparison of Tables 6 and 7 indicate that the measured system resin surface energies lie within expected values for these solutions. DuPont gives the surface energy of the cured Teflon AF 1600, γ_{T}, as 15.7 mJ/m².

Calculation of Voltage Required for Flat Surface: With the surface energies and contact angles in hand, the interfacial energies between the Teflon surface and the system resins may be calculated by rearranging Equation (4). The interfacial tension between Teflon and SPEGDA-258, γ_{T-SPEG258} is given by:

γ_{T}−γ_{SPEG258} Cos θ=γ_{T-SPEG258} (13)

where θ is the measured contact angle of a single droplet of SPEGDA-258 on the Teflon surface. Table 8 shows the interfacial energies for the system resins on the Teflon surface.

Equation (5) may now be rewritten for an Octane/SPEGDA-258/Teflon system as:

$$V = \sqrt{\frac{2 {d}_{f} ( {γ}_{T - SPEG 258} - {γ}_{T - Oct} )}{ɛ}} \qquad ( 14 )$$

With the calculated dielectric permittivity of 3.07e⁻¹¹ F/m, the interfacial energies given in Table 8, and the dielectric film thickness d_{f} given in Table 2, the voltage required to flatten the interface of a given two fluid system can now be calculated. These voltages are given in Table 9.

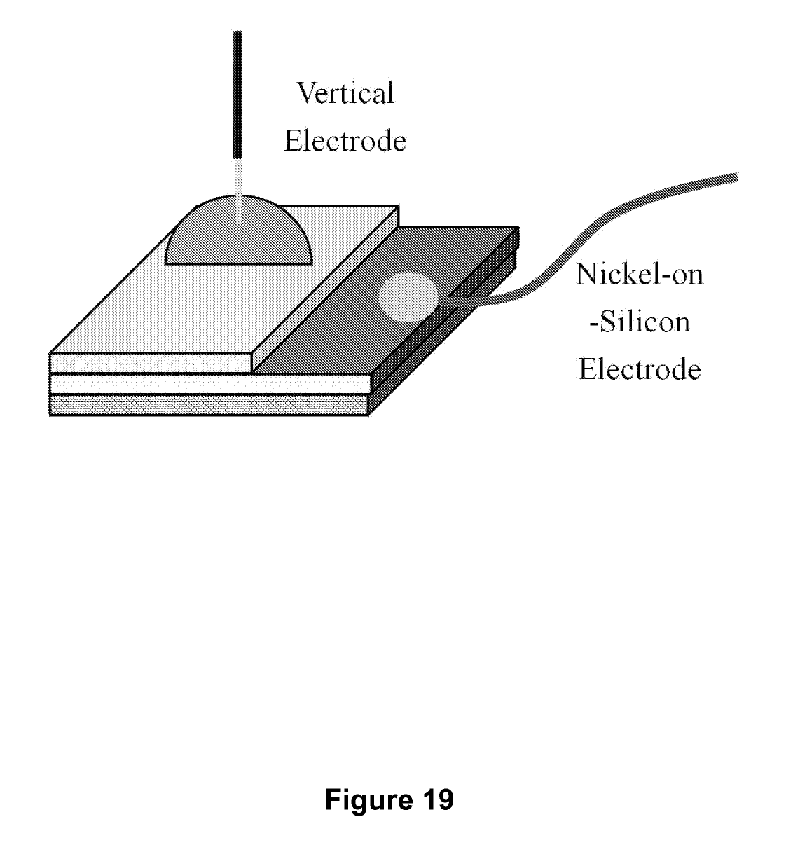

Voltage Response of Various Liquids: The goniometer is also extremely helpful in characterizing the surface response of single droplets to an applied voltage. A single droplet is placed on the surface of one of the electrowetting electrodes described above and a second electrode is inserted into the center top of the droplet surface. When a voltage is applied, charge builds up on the liquid-solid interface and the surface energy of the interface is altered, thus modifying the contact angle of the liquid with the substrate. The experimental set-up is shown in FIG. 19. The single drop rests on top of the Teflon layer (crosshatch). The nickel electrode, connected to the voltage source by soldered copper wire, is sandwiched between the Teflon and the silicon substrate (brick).

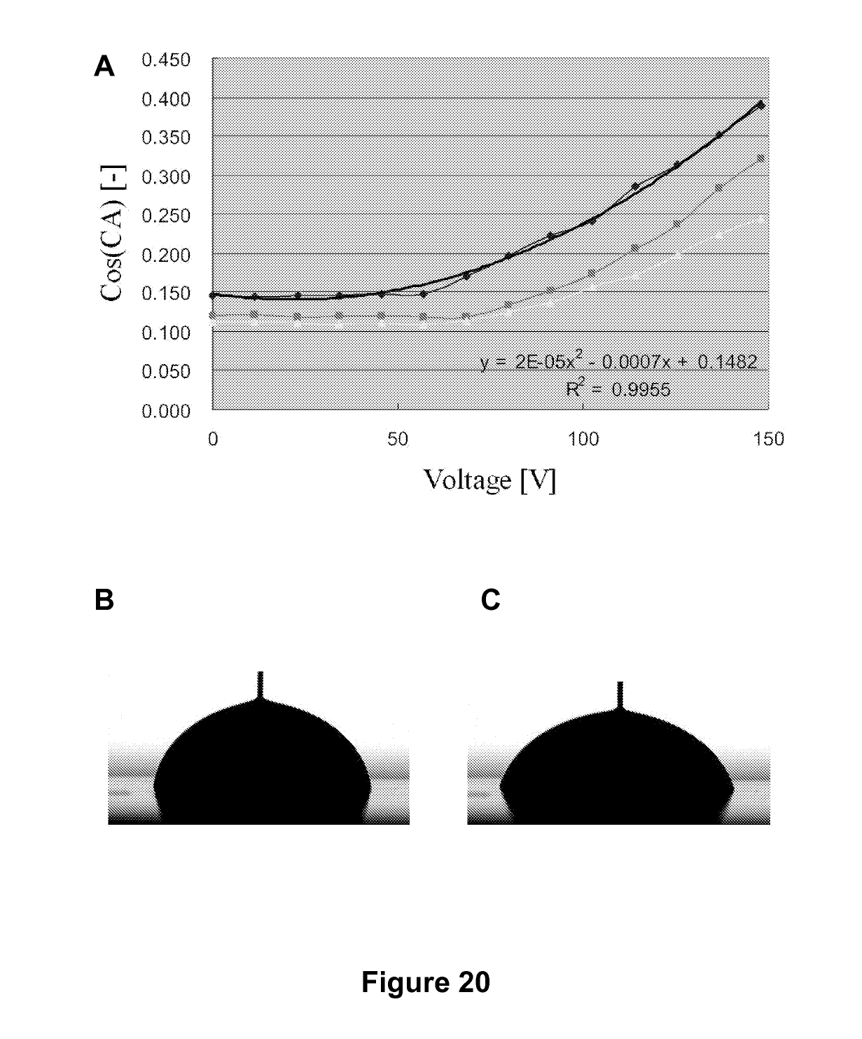

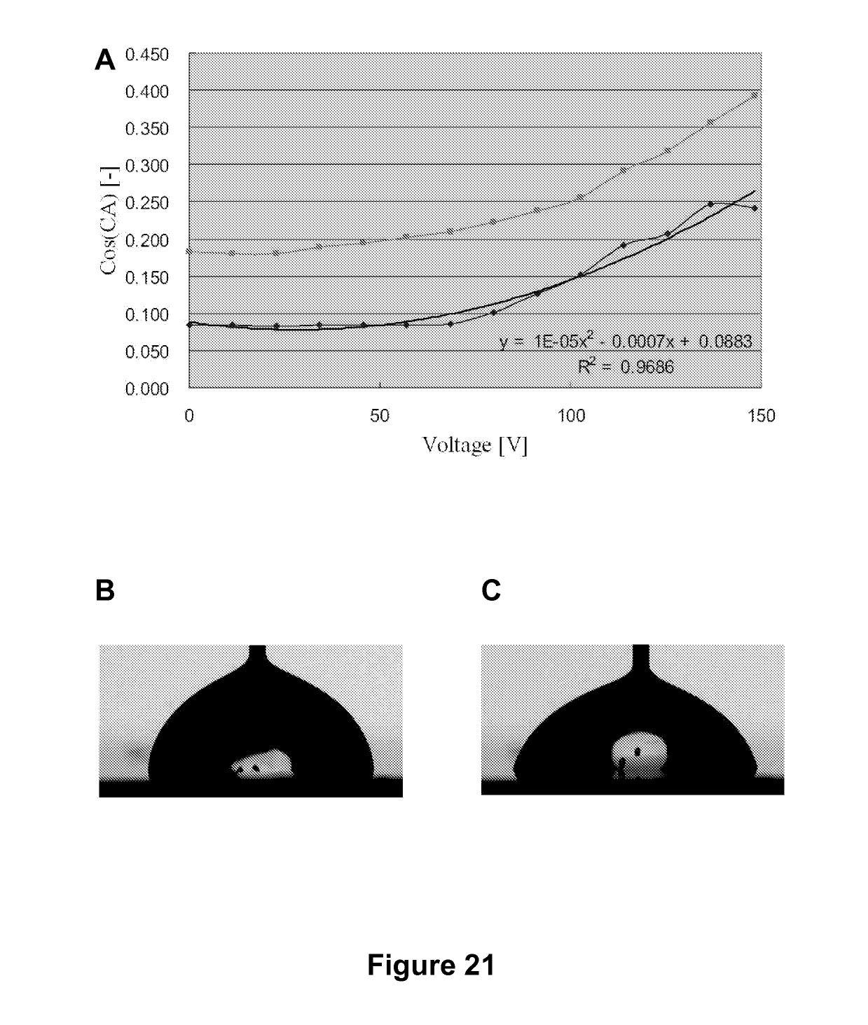

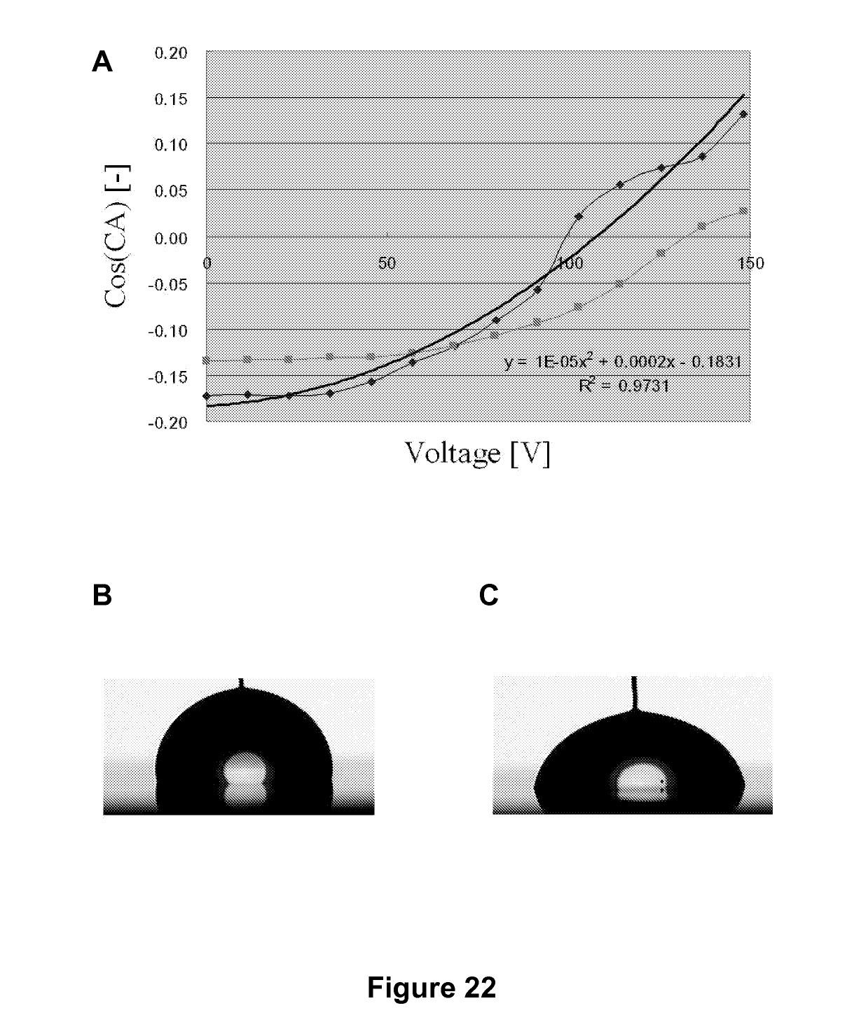

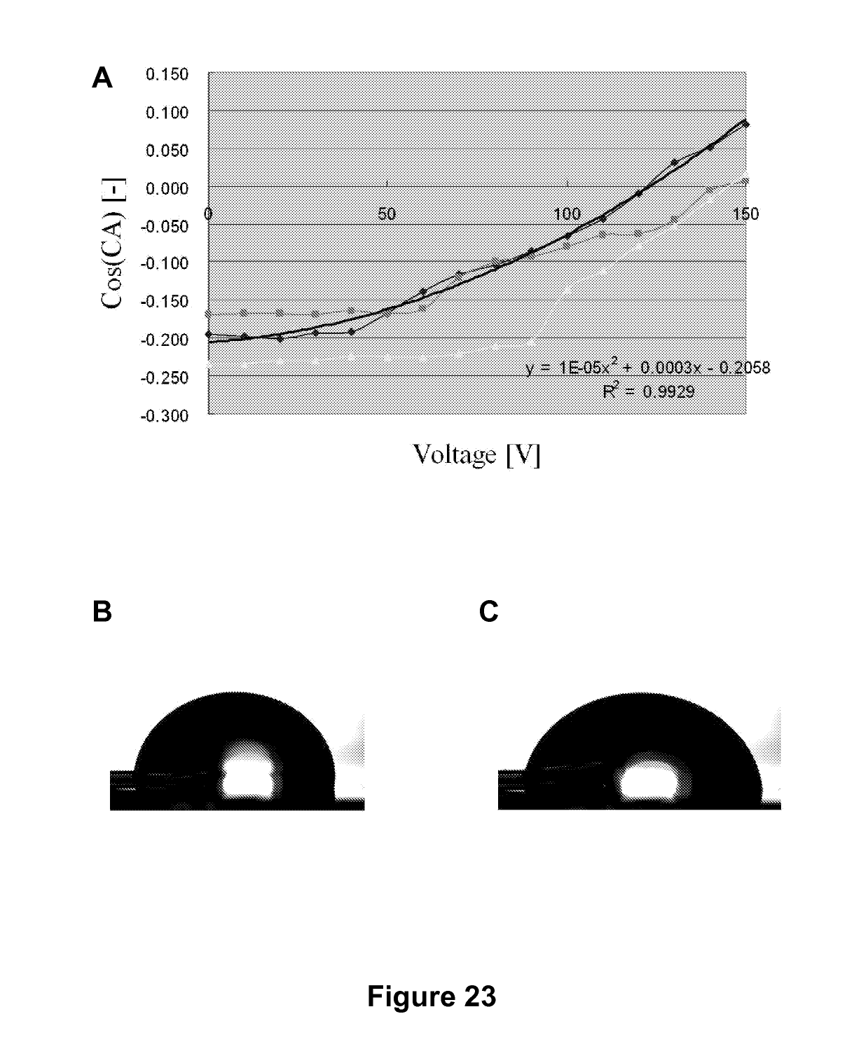

The droplets were deposited on the Teflon surface via a Fisher micropipetter suspended above the electrode to ensure that the droplet size of 6.0 μL was consistent between experiments. Once the droplet was in position the vertical electrode was inserted from above. The voltage between the two electrodes was then varied in a voltage sweep, generated by the Keithley Picoammeter, from 0V to 150V in 50 seconds. The contact angle modification for the electrowetting monomers are given in FIGS. 20-23.

The contact angle response is clearly proportional to V² in all cases, but it shows considerable hysteresis from trial to trial as to how the contact angle changes under the same applied voltage. As the droplets were tested on different portions of the Teflon surface, this error is best explained by the variations in the local Teflon surface morphology. Further error may also be caused by the surface distortion caused by the insertion of the vertical wire electrode into the droplet.

The data presented above supports the proposition that creating a given contact angle condition is a more complicated endeavor than simply applying a single voltage to a droplet. The large amount of hysteresis present demands a sophisticated control system capable of forward and backward voltage application to coax the droplet into the correct shape in circumstances where the surface conditions of the electrodes cannot be precisely quantified or controlled.

Enhanced PμSL via Two Fluid Electrowetting

As interesting as single droplet information may be for the characterization of the individual material response to an applied voltage, successful fabrication of microstereolithography components requires a two fluid system capable of enforcing a flat two fluid interface over the substrate.

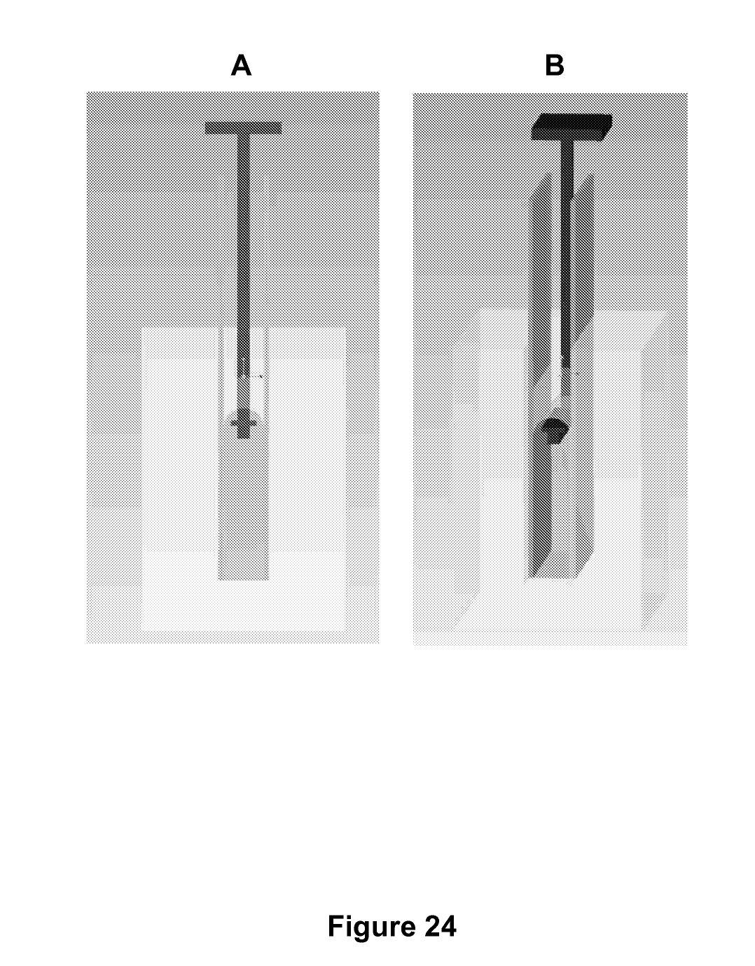

The design of the two fluid chamber for enhanced projection microstereolithography is shown in FIG. 24. The two vertical electrodes face each other supported by the channel walls. The channel itself made from two milled PDMS halves cemented together with JB Weld, a common commercial cold weld. The completed chamber was filled with liquid Teflon AF 1600 and then drained to coat all the interior surfaces with Teflon. The chamber was cured according to the same recipe used for the electrodes as described in Table 2. The result is that both fluids see the same surface condition whether the fluids are in contact with the chamber or the electrodes. The common surface condition greatly aides in observation of the electrowetting phenomenon as well as reducing unnecessary interface distortion that affect the experimental results. The channel width is 0.5 inches. The two immiscible fluids rest in the chamber gap between the electrodes. The conductive fluid on the chamber bottom is SPEGDA-575, and the nonconductive fluid on top is octane.

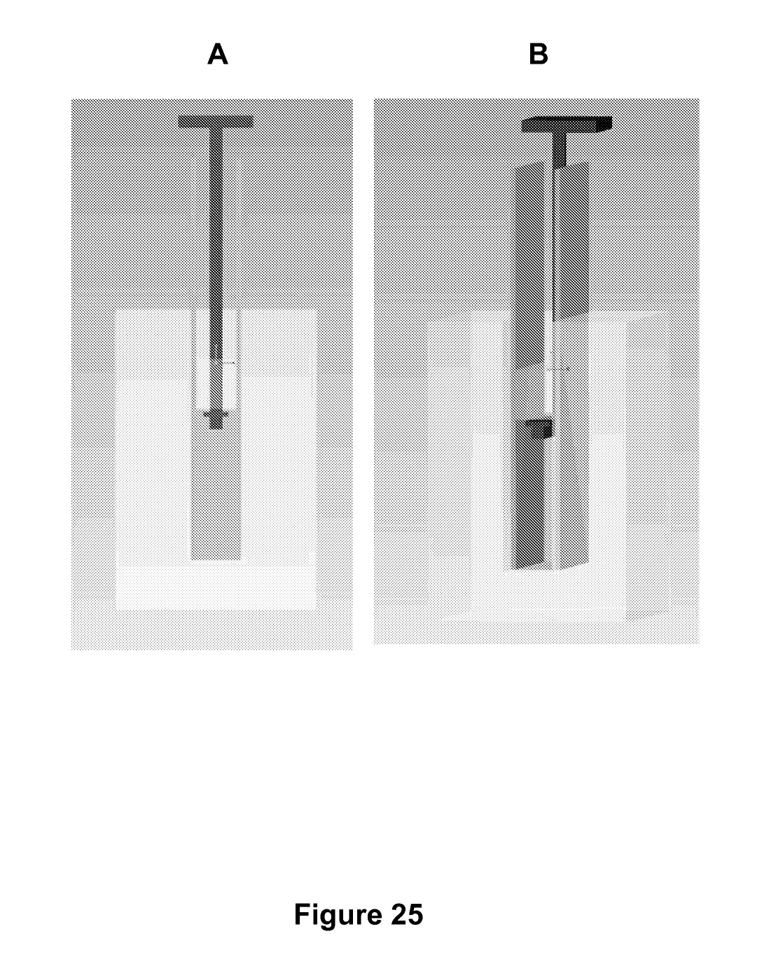

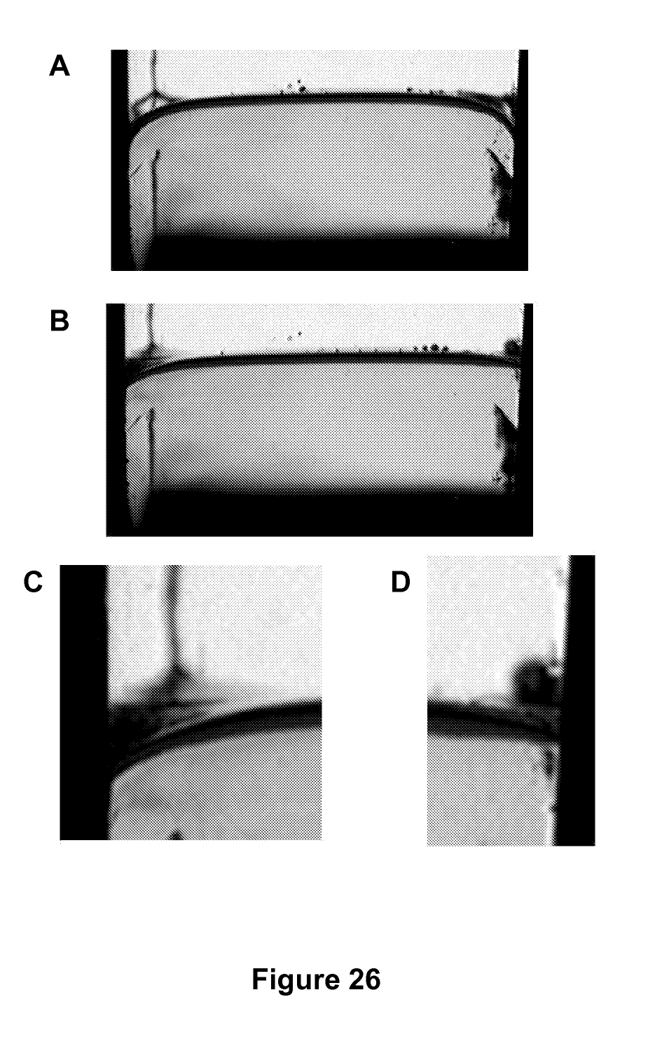

Enhanced Projection Microstereolithography Fabrication. Applying the Flattening Voltage When voltage is applied across the two electrodes, the menisci of the nonconductive and conductive fluids on the interfacial boundary will flatten according to Equation (6). FIG. 25 shows a concept view of the two-fluid meniscus when the proper flattening voltage is applied in contrast to the curved two-fluid meniscus depicted in FIG. 24. FIG. 26 shows experimental images of the two-fluid meniscus in both at-rest and flattened conditions. The flattening is most visible at the electrode-fluid interface along the channel walls (FIG. 26C-D). For the system shown in FIG. 26 the deflection of the meniscus at the center of the channel from A to B is 220 μm.

The sample holder is immersed in the channel at a height of one layer thickness below the flat surface. When the surface reaches a zero-degree contact angle, the system is ready for fabrication. Accordingly, electrowetting provides a means for reducing the surface dwell time, thereby decreasing single layer fabrication time in accordance with Equation (2).

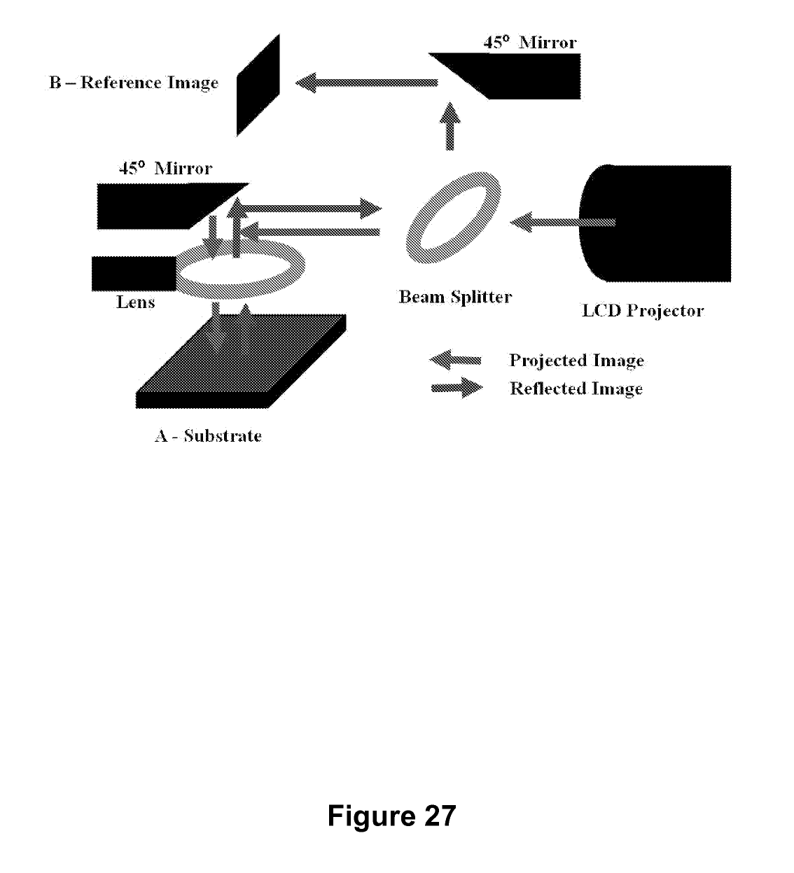

Image Focusing: Focusing the light on the substrate surface through the two liquids requires an optical set-up similar to the system diagramed in FIG. 27. In order to determine the initial focused position of the substrate, the sample stage elevator is translated vertically until the image on the substrate is in full focus. The optical path to the focus image plane (blue) must then be set such that the reference image at position B is also in focus. The two fluids are then added to the fluid chamber so that the substrate lies underneath the two fluid boundary; the fluids will change the optical path lengths from the projector to both images. After the fluid is flattened by applying the necessary voltage, the projector focus is adjusted to give a clear image on the reference image plane at position B. This adjustment assures that the image on the substrate is also in focus. With the system properly focused; the light source is activated for the required time to fully expose the PEDGA monomer photoactive material.

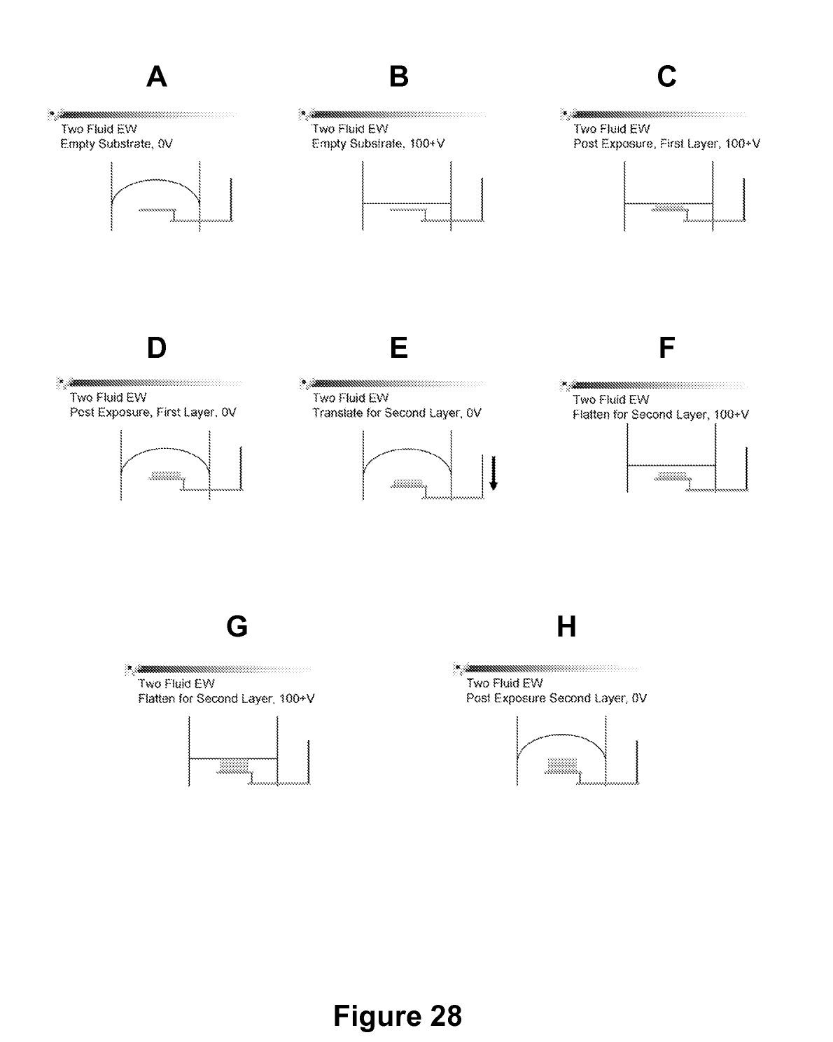

Multilayer Components: For multilayer components, the fabrication proceeds as detailed in FIG. 28. Once the substrate is in the proper position and the image is focused as described above (FIG. 28B) the projector is activated and the layer is polymerized (FIG. 28C). The voltage is then released (FIG. 28D) and the stage translated downward (FIG. 28E) to make room for the next layer; once the stage is in the correct position the voltage is reapplied (FIG. 28F). When the second layer is polymerized (FIG. 28G), the voltage is released (FIG. 28H) and steps E-H are repeated for the necessary number of layers. In this manner each layer's surface is forced via electrowetting to flatten. The larger significance of this enhancement is that surface control of the fabrication layer no longer depends on waiting for gravity to overcome the monomer's viscous forces. The dwell time between steps is now dependent only on the electrowetting response time for the given fluid system, which can be significantly less than the time required for gravity to provide a flat surface.

Enhanced PμSL Results

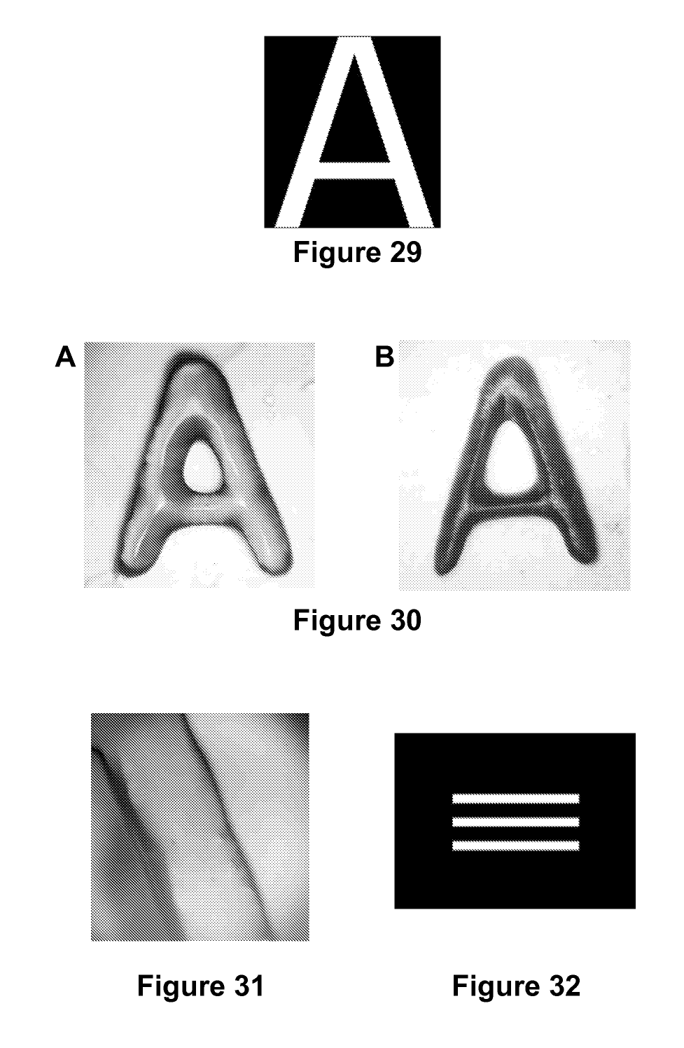

The black and white “A” pattern shown in FIG. 29 is used as a bitmap mask for testing the electrowetting-enhanced projection stereolithography system. FIG. 30A shows the electrowetting and non-electrowetting “A” samples with otherwise identical fabrication conditions at 5× magnification. The non-electrowetting sample in FIG. 30B is visibly thinner over the whole sample area. Its layer thickness profile is also triangular; the top of the layer, which was in contact with the two layer interface, is much narrower than the bottom of the layer. In contrast, as shown in FIG. 31, the electrowetting sample displays a much more rectangular profile, indicating a more precise light focus caused by a flatter meniscus interface across the sample area. Accordingly, not only can electrowetting decrease fabrication times, but electrowetting can provide higher quality three-dimensional structures having improved resolution and pattern control. Both samples were fabricated with ten second exposure times.

Multilayer “A” structures are also fabricated. Due to the resolution limitations of the crude x-y-z stages employed in this enhanced PμSL setup, the successive layers of the multilayer A′ s did not lie directly on top of one another. Nevertheless, the layer thicknesses are easily measurable using the Philips XL30 environmental scanning electron microscope. The three layers measured 185, 180, & 192 μm respectively, or an average of 185 μm with a 4% standard deviation. These individual layers are fabricated with a dwell time of only five seconds between them, as compared to 10-20 seconds with a standard PμSL setup. The five second dwell time allowed for all visible fluid motion on the two fluid interface to cease before fabrication was begun.

Additionally, a single layer component comprising three identical bars (FIG. 32) is fabricated; their width filled roughly ⅙ of the two-fluid channel. By measuring their relative heights, the flatness of the two-fluid layer may be inferred. Optical profilometer scans of the fabricated component yielded poor quality results, but the three bars have maximum normalized heights of 0.94, 1, and 0.86, respectively, and a standard deviation of 8%.

These results confirm that standard photopolymerizable polymers, such as PEGDA monomers, can be effectively polymerized in a two fluid microstereolithography system. Furthermore, the phenomenon of electrowetting is used to effectively induce a flat monomer surface suitable for fabrication of microstereolithography components. Finally, and most importantly, electrowetting is shown to decrease the dwell time between the fabrication of successive layers up to 50% over standard free surface dwell times. This work was performed using relatively crude positioning stages; incorporation of electrowetting into the PμSL system detailed in Example 1 provides further analysis of electrowetting's effect on basic microstereolithography metrics of resolution as well as further dwell time comparisons.

### Example 3

### Modeling Biological Tissue

If the structure and function of biological systems can be better understood, their engineering knowledge can be more effectively utilized to serve humanity's needs. The most effective guides to utilizing the engineering designs of living systems are the living systems themselves.

One of the challenges involved in accurately modeling biological systems is reproducing the fine shades of density, structure, and function present in biological tissues. Tuning the properties of different regions of the tissue samples is critical to proper modeling, yet very difficult to accomplish using traditional manufacturing techniques. Here the modeling of biological systems in PEDGA monomers and the ability of microstereolithography to “tune” the diffusion properties of such models are demonstrated. With these tools in hand more accurate models of biological structures can be realized, and their unique properties may be utilized to serve a broad range of applications ranging from materials production (e.g., biologics, drugs, biofuels) to microvascular network and tissue implants. This tuning is accomplished with a further enhancement to microstereolithography: incorporating grayscale lithography into the fabrication process.

Modeling Material: Polyethylene Glycol Diacylrate

Polyethylene glycol diacylrate (PEGDA) is a water-soluble functional comonomer commonly used in flexible plastics, varnishes, and dental compounds. A well-defined biocompatible polymer, the chemical structure of PEGDA is:

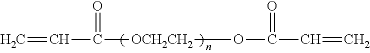

PEGDA is available commercially at a variety of molecular weights. The molecular weight of the base liquid starting material is selected depending on the desired property of the end product. For the examples presented herein, a monomer with an average molecular weight of 575 is used.

Synthesis of PEGDA-575 solutions for use in projection microstereolithography systems is straightforward. A desired volume of PEGDA is combined with a photoinitiator and an absorber by gentle stirring for several hours at 30° C. on a standard magnetic hotplate. Photoinitiator Irgacure 819 from Ciba and absorber Sudan I from Sigma-Aldrich are added to the PEGDA-575 monomer at 2% and 0.5% by weight, respectively. The absorber concentration is a critical variable for the vertical resolution of the final component. In this case 0.5% Sudan I reliably allows layer thickness from 10 to 50 μm; samples with thinner or thicker layers require lower or higher absorber concentrations.

Model Fabrication: In order to correctly describe the diffusion characteristics of biological tissue, an accurate model is first made. A common housefly wing is chosen as modeling template based on two factors: (1) The flywing has an extensive network of hollow microtubules that give it strength and conduct oxygen to the wing's tissues. The three dimensional microfluidic network is an excellent example of a system that microstereolithography is uniquely well-positioned to recreate in that it is readily imaged; (2) Samples are plentiful, cheap, and readily available. However, given sufficient information regarding size and geometry of a network, any network can be reproduced by the systems and methods disclosed herein.

CT Scanning: In order to create a physical model of a biological system with microstereolithography, a CAD model must first be developed. The source data for this CAD model can be acquired through the use of a high-resolution CT scanner. CT scanners, used by thousands of hospitals all over the world to analyze the structure of human bodies, provide extensive dimensional data about the composition of biological organisms. Standard CT scanners image tissue on a scale too large to discern microscale features, but micro-CT systems are capable of imaging biological tissue with microscale resolution.

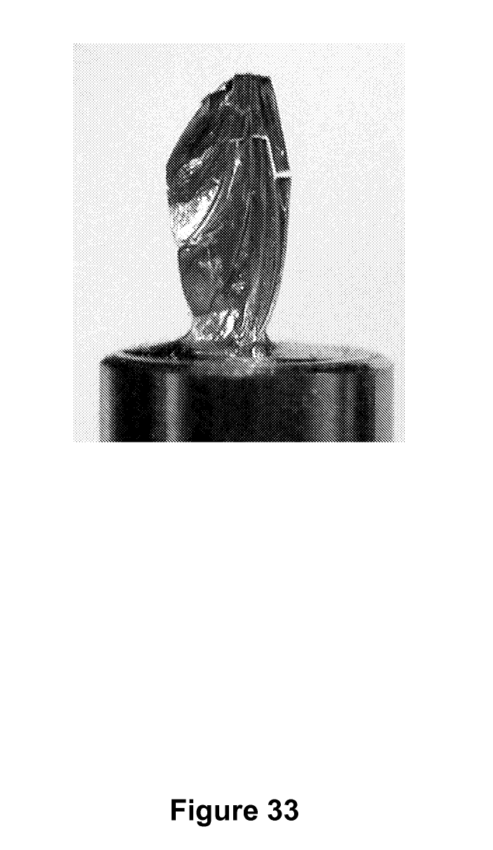

Sample Preparation: The housefly wing is preserved in an ethanol bath after capture. Prior to mounting, it is soaked in a solution of iodine overnight and then sputtered with for 240 seconds with gold-palladium to increase the tissue's absorption of X-rays. FIG. 33 shows the flywing mounted on the end of a brass post to ensure the scanner full line-of-sight access to the sample. The flywing is approximately 5.5 mm tall by 3.0 mm wide at its extreme points.

Micro-CT Scans: A SkyScan 1172 Desktop X-Ray Microtomograph, scans the sample. The microtomograph has a spatial resolution of 5 μm and a voxel size of 1×10⁻⁷ mm³. The system produces two dimensional shadow images of the three dimensional object. Each pixel of these scans contains the aggregate absorption information along the scan path. Dense tissues absorb the X-rays and appear as white areas. Softer tissues allow more of the radiation to pass through and appear in a shade of gray. The scans are taken at a fixed radial distance from the object at intervals of tenths of a degree. By sampling at 0.3 degrees, one thousand images can be collected in about 30 minutes. The scans are stored as 16 bit TIFF images.

Image Reconstruction and Formatting: The scans are taken in a sagittal plane with the vertical axis of the object parallel to the image plane and contain aggregated absorption information. In order to localize the specific absorption of individual voxels along the scan path, multiple images are compared. FIG. 34 diagrams how individual absorption points can be identified in a reconstructed transverse image by comparing multiple sagittal scans. (In a transverse image the vertical axis of the real object is oriented perpendicular to the image plane; in a sagittal image the vertical axis of the real object is oriented parallel to the image plane). NRecon image reconstruction software is used for this purpose. Increasing the number of scans increases the resolution and accuracy of the reconstructed images. Images are reconstructed by assembling layers of data with a thickness that is constant throughout the height of the scanned object.

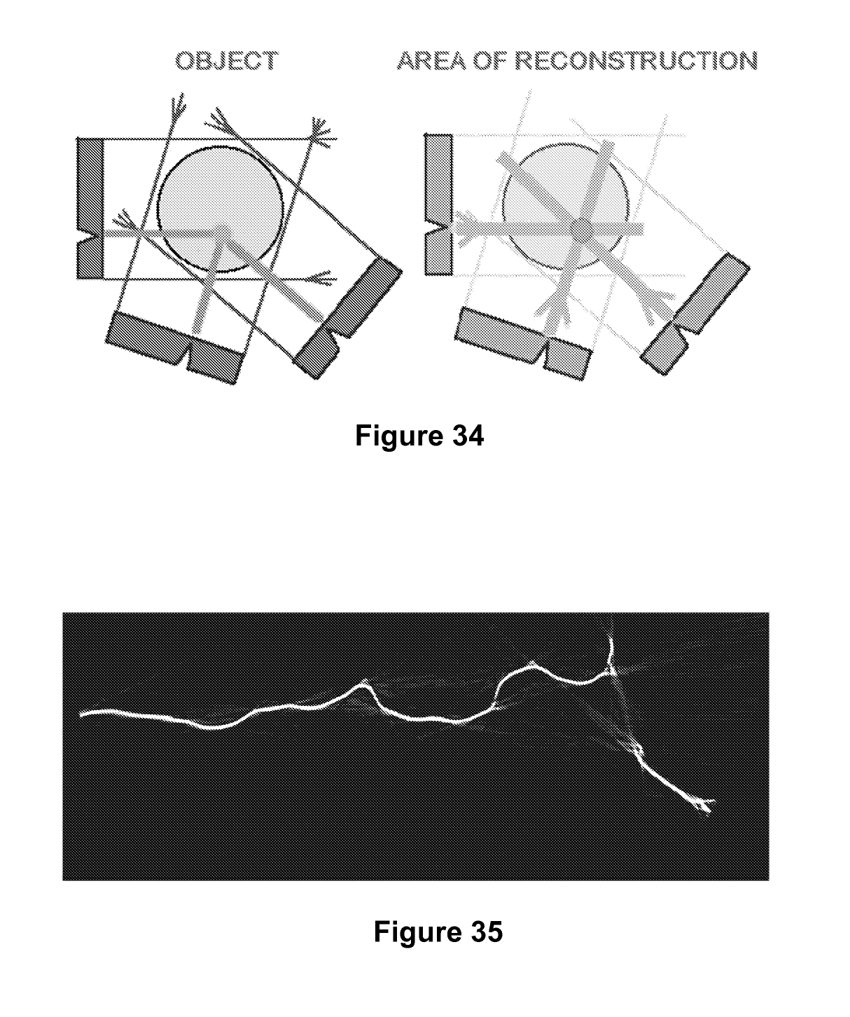

The X-ray source will emit X-rays in a conical distribution, so the reconstruction software employs a Feldkamp correction algorithm to properly correct for the conical projection object's voxels present in the original scans [40]. The result is a set of 8-bit (256 grayscale) bitmap transverse images of the original object that display its three dimensional structure as seen by the absorbed X-rays. FIG. 35 shows an example reconstructed image of a flywing cross-section. The resulting transverse images are highly valuable for microstereolithography because it requires just such a set of cross-sectional images in order to fabricate its three dimensional structures.

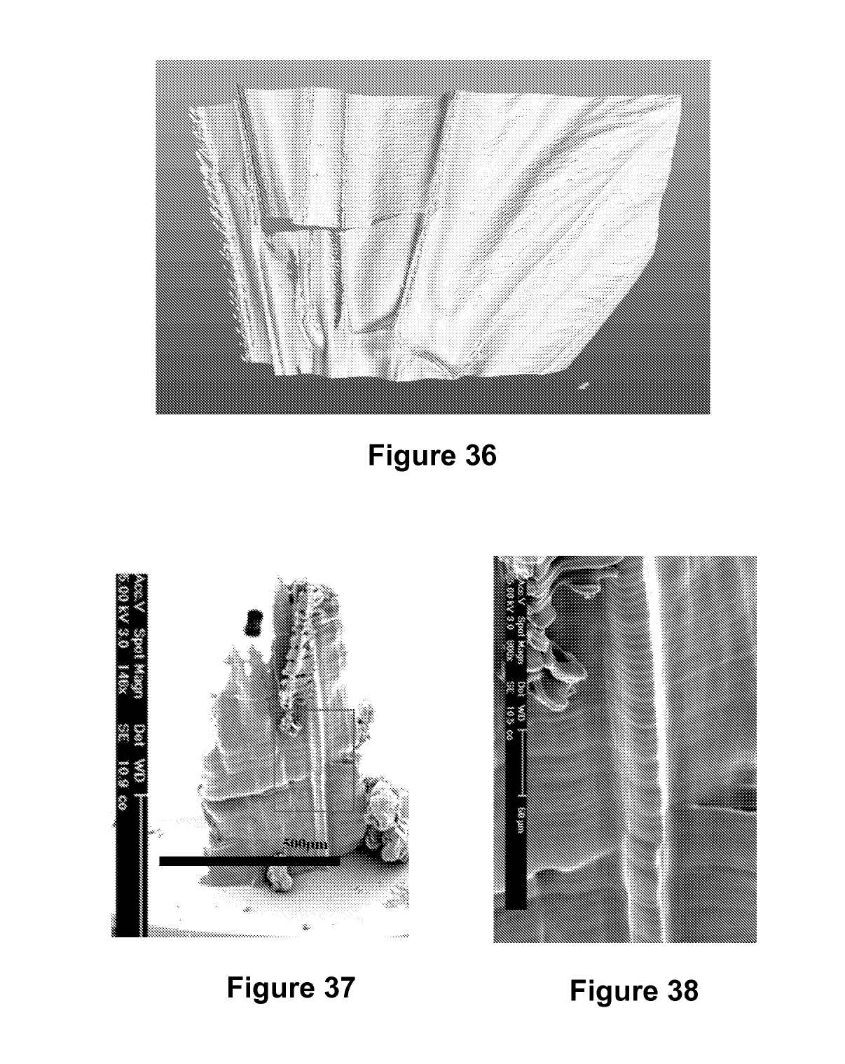

Image Editing: Amira® is a powerful 3D modeling software that allows for detailed filtering of the image data. It can recreate three dimensional surfaces from the reconstructed two dimensional bitmaps, as shown in FIG. 36. The 3D models are extremely helpful to the user in verifying the quality of the sample data and identifying specific regions of the model that may be of further interest.

However, since the projection stereolithography system described in Example 1 already takes 2D bitmap slides as inputs, the Amira® image editor's most valuable contributions are its extensive image processing capabilities. Noise artifacts can be removed from the final images and the brightness and contrast of individual features can be adjusted on an image-by-image basis. Amira® provides extensive thresholding capabilities that helps overcome some of scanning gaps inherent in the CT rendering of medium density tissues. Furthermore, secondary support structures not present in the original tissue may be added if desired. When all of the necessary image filtering is complete, the desired transverse images, corresponding to specific layers of the PEGDA-575 model, may be exported. These images are directly employed by the projection microstereolithography system as dynamic masks.

Replication of 3D Flywing in PEGDA-575: The flywing structure is particularly interesting to engineers because of its complex structure. The hollow network of microtubules gives strength and flexibility to the wing while allowing for oxygen transport throughout the structure and into the wing tissue. Therefore the main microtubule running along the center of the wing segment is chosen as the first feature for modeling. FIG. 37 shows a PEGDA-575 model of a section of the housefly wing surrounding the main microtubule that was fabricated by the projection microstereolithography system. It has 250 layers each with a thickness of 10 μm. The upper sections of the wing proved to be too thin to survive removal from the PμSL system and the subsequent drying process, even though supercritical drying with carbon dioxide was employed. Thus, only about 25% of the intended wing structure appears in FIG. 37. Due to the limitations of the PμSL system the flywing segment as fabricated here is rendered at ⅓ scale. FIG. 38 shows the boxed area of FIG. 37; the exterior of the flywing's main microtubule, a triangularly shaped channel approximately 40 μm across.

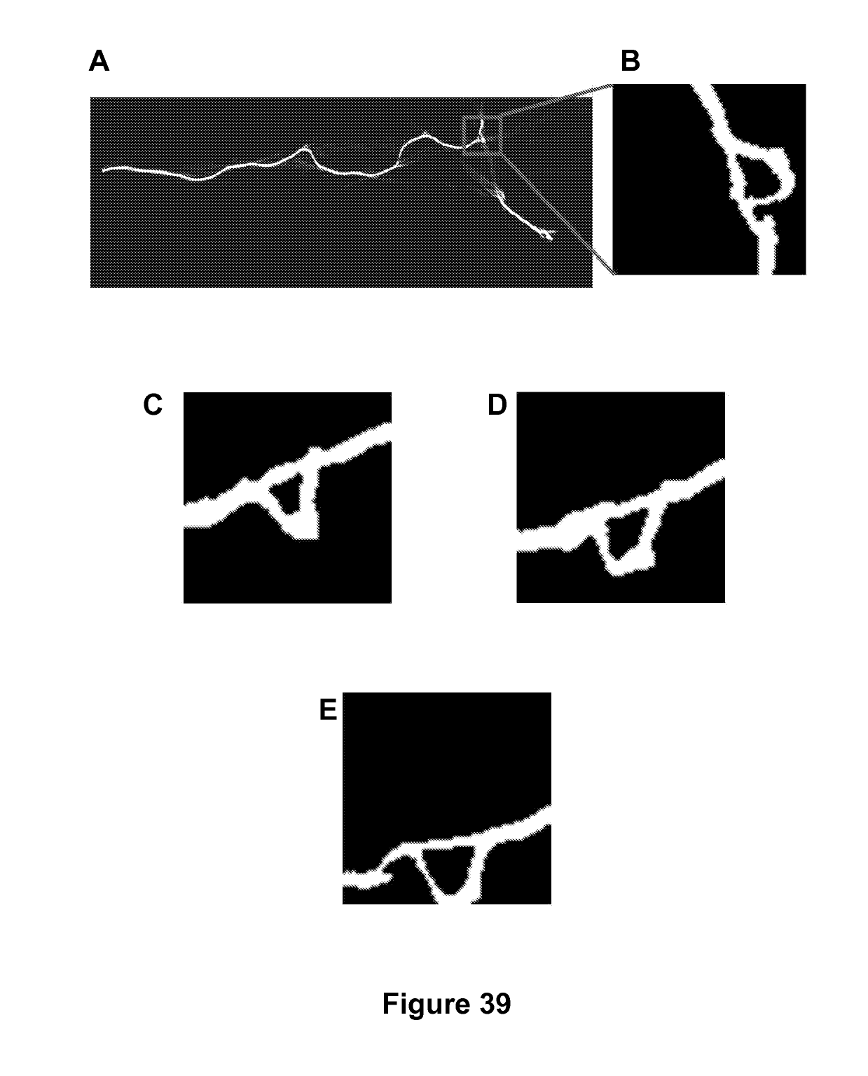

In order to further isolate the microchannel, the bitmap images to be used as masks by the PμSL system may be modified. By selective cropping of the necessary layers the desired feature can be isolated and enlarged. FIG. 38 shows the area that was cropped from the original bitmap images to fabricate an isolated model of the main flywing microchannel. As shown in FIG. 39C-E the microchannel cross-sections vary considerably in shape and position relative to each other. These slices hint at the complex 3D geometry of the flywing.

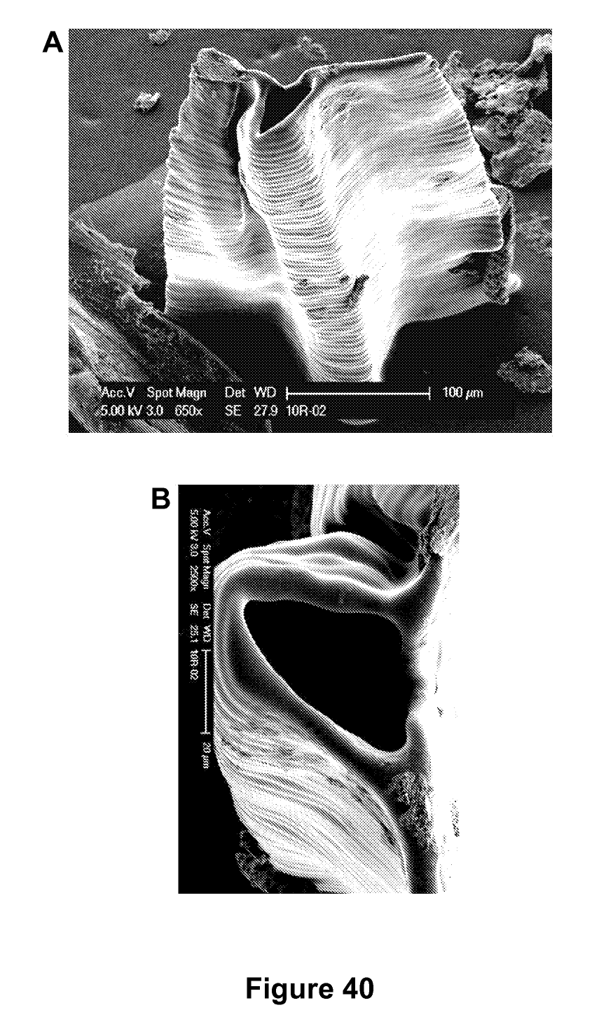

These cropped microchannel images are sized such that their PEDGA model is rendered at true scale. FIG. 40 shows the three dimensional PEGDA-575 model of a segment of the main flywing microchannel. The model clearly displays the multi-axis variation in the flywing geometry along its Z-axis. This true scale model can justifiably claim to accurately represent the original flywing structure within the limits of the micro-CT's 5 μm per voxel resolution. (The resolution of the PμSL system is considerably better at 1.1 μm per pixel).

### Example 4

### Enhanced uSL by Gray Scale Lithography

Grayscale Fabrication for Tunable Structures: Representing the physical shape of a biological structure is a significant achievement. However, extracting functional benefits from Nature's designs requires that the structure's properties be replicated as well. The flywing's microtubule network, in addition to providing mechanical support for the wing also delivers oxygen to the surrounding tissue by means of diffusion. Clearly, diffusion does not take place at the same rate throughout the network, so a functional replication of the microtubule network requires that the diffusion properties of the PEGDA-575 be variable throughout the structure. Toward that end a grayscale stereolithography scheme is described.

Grayscale Profile Height Variation: Normal microstereolithography components are made using black and white bitmap images that serve as dynamic masks for the successive layers. However, if grayscale shades are used in the bitmap mask, intermediate light intensities are capable of being transmitted to the liquid surface. This staggered intensity can either create features with varying heights or cross-linking densities within a single layer thickness.

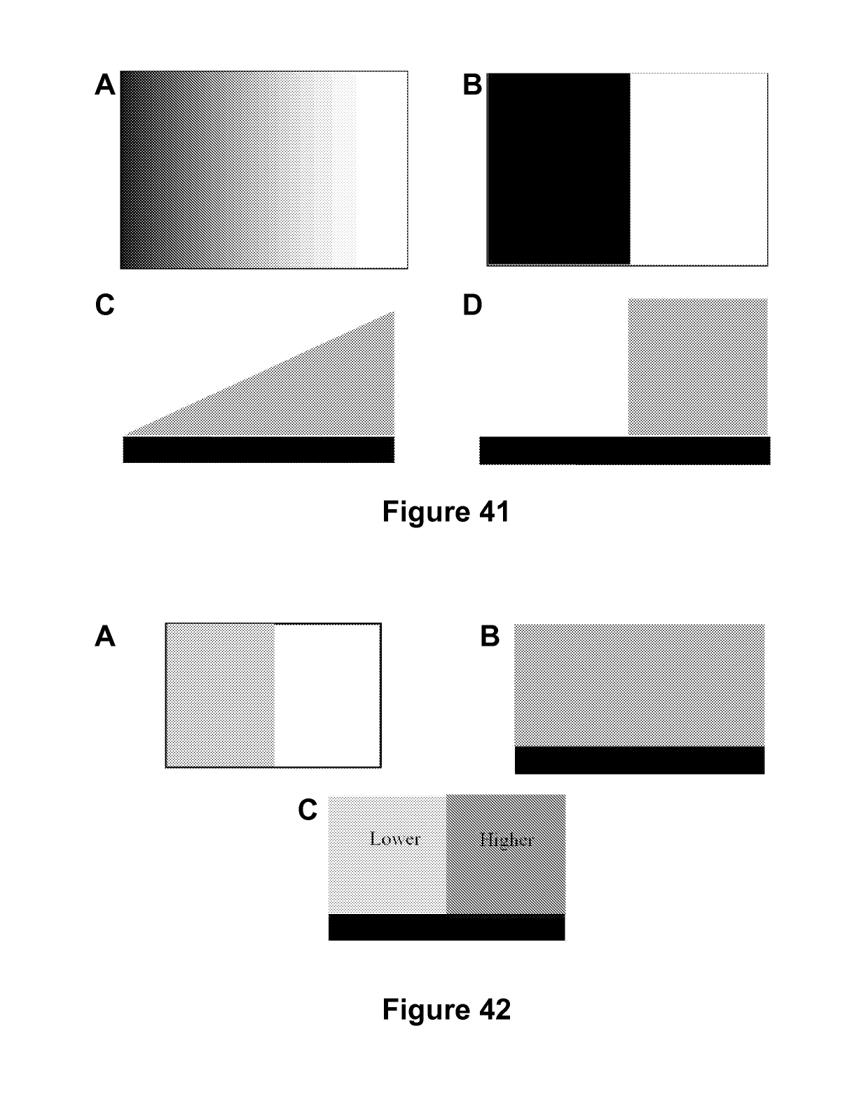

FIG. 41C shows the height profile variation produced within a single layer thickness when a linear grayscale gradient (FIG. 41A) is employed as a mask, provided that the light intensity in the gray regions is insufficient to polymerize the entire layer thickness. FIG. 410 shows the results for the same layer thickness using the black and white only mask of FIG. 41B. Eight-bit, 256 shade grayscale fabrication drastically reduces the digital limitations on the object shape as a result of a discrete layer thickness.

Grayscale Profile Cross-Linking Variation: Furthermore, in the case that the light intensity of the PμSL system is set such that the intensity in the gray region is sufficient to polymerize the entire layer, the physical appearance of the two regions would be identical (FIG. 42B), but they would in fact have different cross-linking densities in their respective regions due to the differing amounts of light introduced and absorbed by the respective regions (FIG. 42C). The different crosslinking densities correspond to different diffusion coefficients for the various regions, as increased polymer crosslinking likewise increases the resistance to molecular motion within the polymer matrix. Thus, grayscale fabrication can be used to tune the regions of a PEGDA model to match variations in a biological tissue's diffusion properties. In addition, different cross-linking densities result in different etch rates so that subsequent processing steps by etching provides further geometric control.

Grayscale Bitmap Creation: The primary challenge in utilizing grayscale fabrication is the creation of the bitmap images for use as dynamic masks. Each pixel's grayscale value is critical to the final structure, and for tall models upwards of 300 images may be employed as dynamic masks. Thus, Matlab is employed to generate the bitmaps under the control of a custom code written to assign the appropriate grayscale values to each bitmap image.

For a given grayscale image of N×N pixels, the grayscale shades may be represented by an N×N matrix, G(x, y), whose elements are integer values ranging from 0 to 255, with 0 corresponding to black and 255 corresponding to white. The Beer-Lambert law, giving the light intensity at a given depth of monomer, is:

$$E ( z ) = {E}_{o} exp ( \frac{- z ( x , y )}{{D}_{P}} ) \qquad ( 15 )$$

where: z is the depth into the monomer; Eₒ is the critical exposure required for polymerization; Dₚ is penetration depth of the monomer.

The z value here also represents the polymerization depth in the monomer for all E(z)>Eₒ. Rearranging for z, the local height of the model in a location corresponding to a single pixel with coordinates (x,y), this equation is:

$$z ( x , y ) = {D}_{P} ln ( \frac{E ( x , y )}{{E}_{o}} ) \qquad ( 16 )$$

Combining all pixels in the bitmap matrix:

$$D ( x , y ) = {D}_{p} ln ( \frac{E ( x , y )}{{E}_{o}} ) \qquad ( 17 )$$

where D(x,y) is the N×N matrix representation of the set of all heights in the desired object. Additionally, the light intensity of the grayscale mask is given by:

$$E ( x , y ) = {E}_{max} \frac{G ( x , y )}{255} \qquad ( 18 )$$

where Eₘₐₓ is the intensity of the light emitted by the light source. This equation merely says that the light intensity transmitted to the liquid surface is the intensity incident on the LCD chip times the fraction of light reflected off the chip by each grayscale pixel.

Furthermore, the maximum height possible for a polymerized structure, Dₘₐₓ, from a given incident light intensity, Eₘₐₓ, is given, from Eq. 18, by:

$${D}_{max} = {D}_{p} ln ( \frac{{E}_{max}}{{E}_{0}} ) \qquad ( 19 )$$

Rearranging Eq. 17 and Eq. 19 for E(x,y) and Eₘₐₓ gives:

$$\begin{matrix}E ( x , y ) = {E}_{o} exp ( \frac{D ( x , y )}{{D}_{p}} ) & ( 20 ) \\ {E}_{max} = {E}_{o} exp ( \frac{{D}_{max}}{{D}_{p}} ) & ( 21 )\end{matrix}$$

Substituting Eq. 20 and Eq. 21 into Eq. 18 gives:

$$G ( x , y ) = 255 \frac{exp ( \frac{D ( x , y )}{{D}_{p}} )}{exp ( \frac{{D}_{max}}{{D}_{p}} )} \qquad ( 22 )$$

Since the layer thickness Dₘₐₓ is defined by the user, the grayscale map G(x,y) may be calculated from the height profile D(x,y) as long as the penetration depth, Dₚ, of the monomer is known. This derivation is from Wu [41].

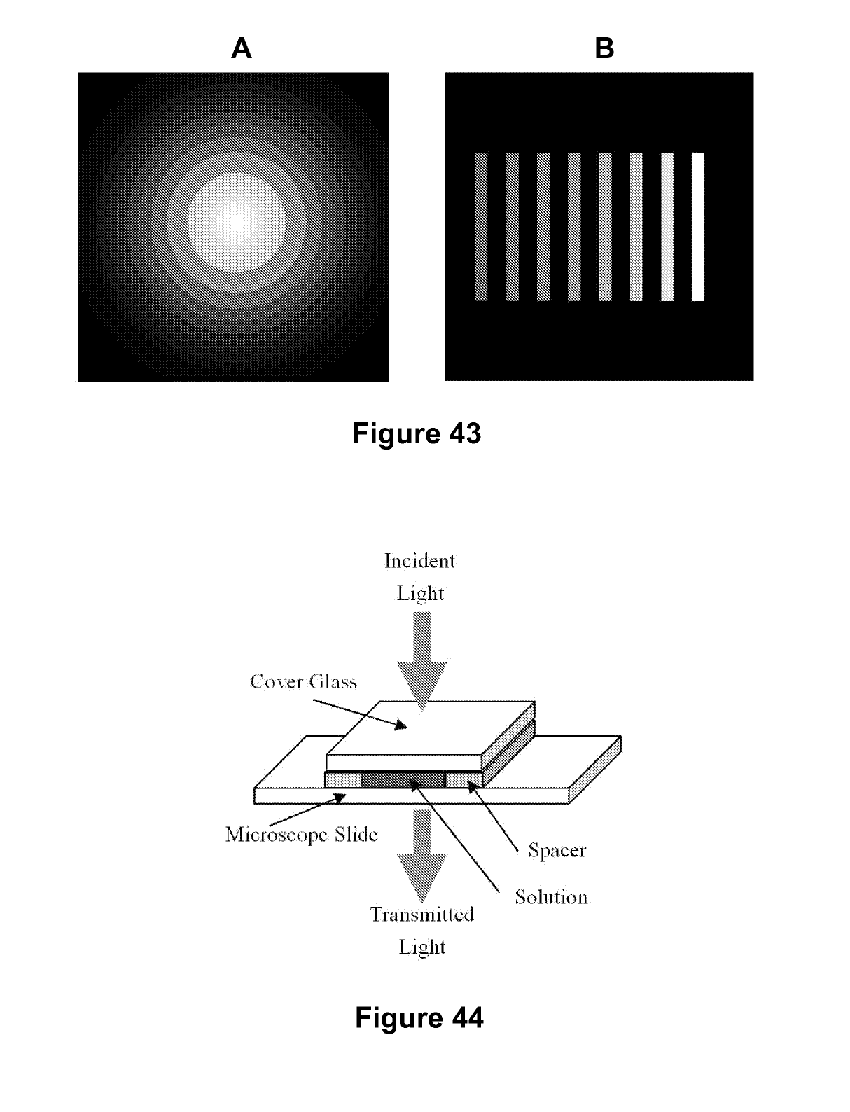

Thus grayscale bitmap images are easily created in Matlab by defining D(x,y) across the matrix and calculating G(x,y) as shown above for the desired Dₘₐₓ and the Dₚ of the monomer solution. A calculated G(x,y) is graphed as a three dimensional scatter plot which is then saved as a bitmap image. If exact model dimensions are vital, additional image processing in Photoshop may be required to properly size the bitmaps. FIG. 43 show examples of grayscale bitmaps created in this manner.

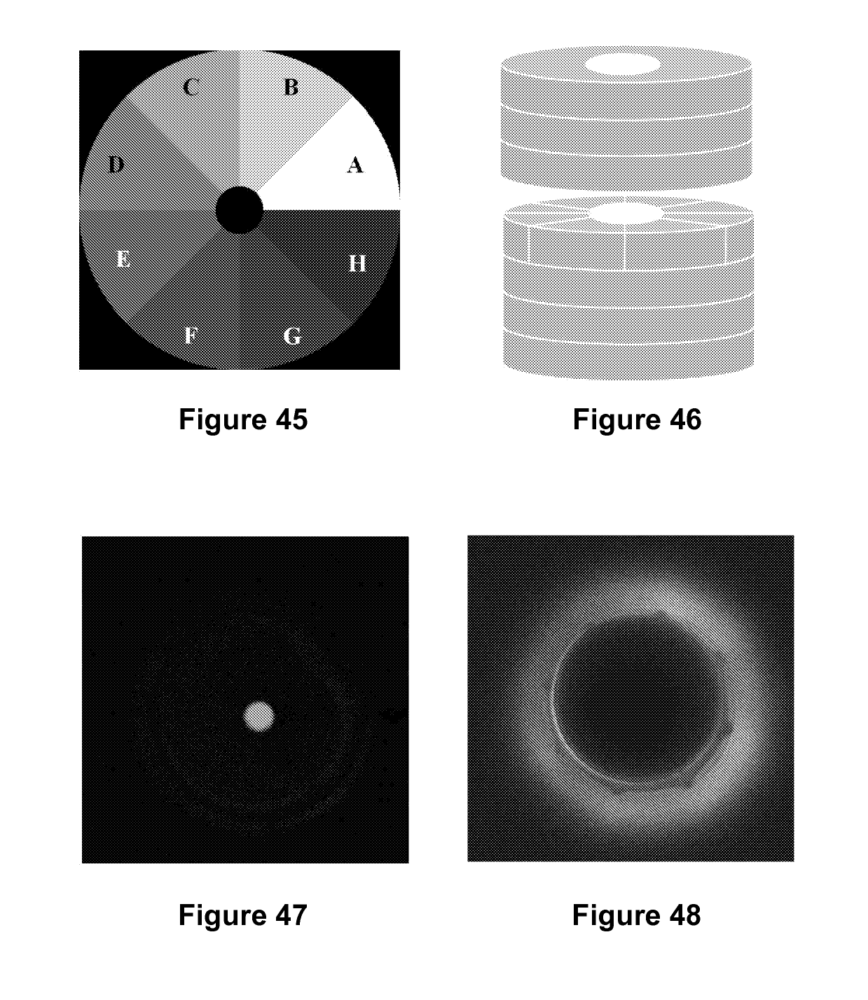

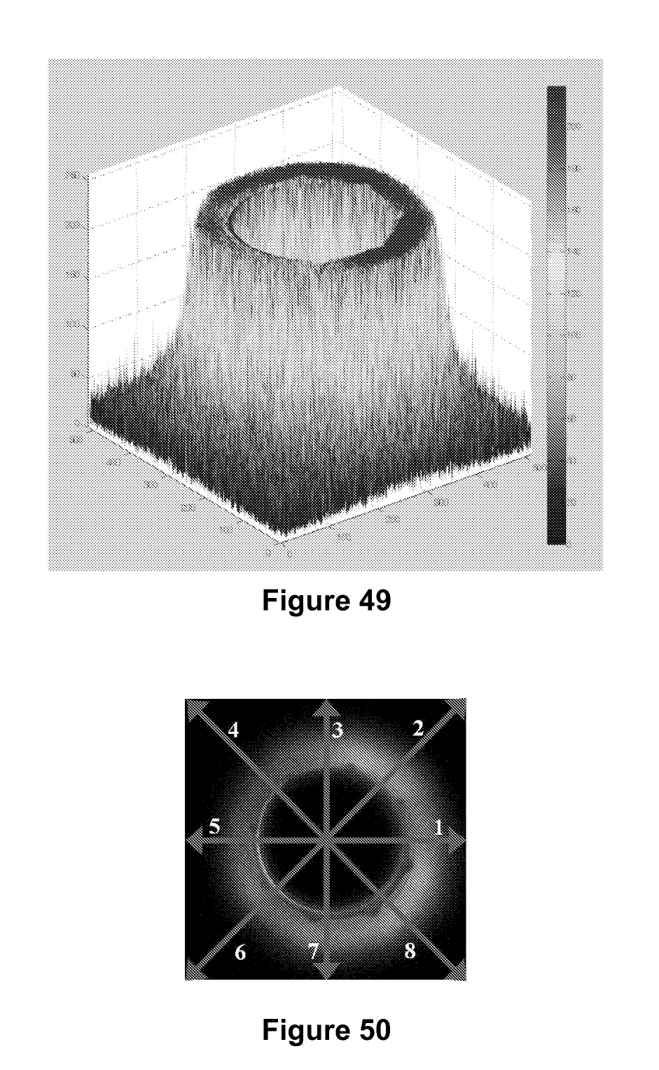

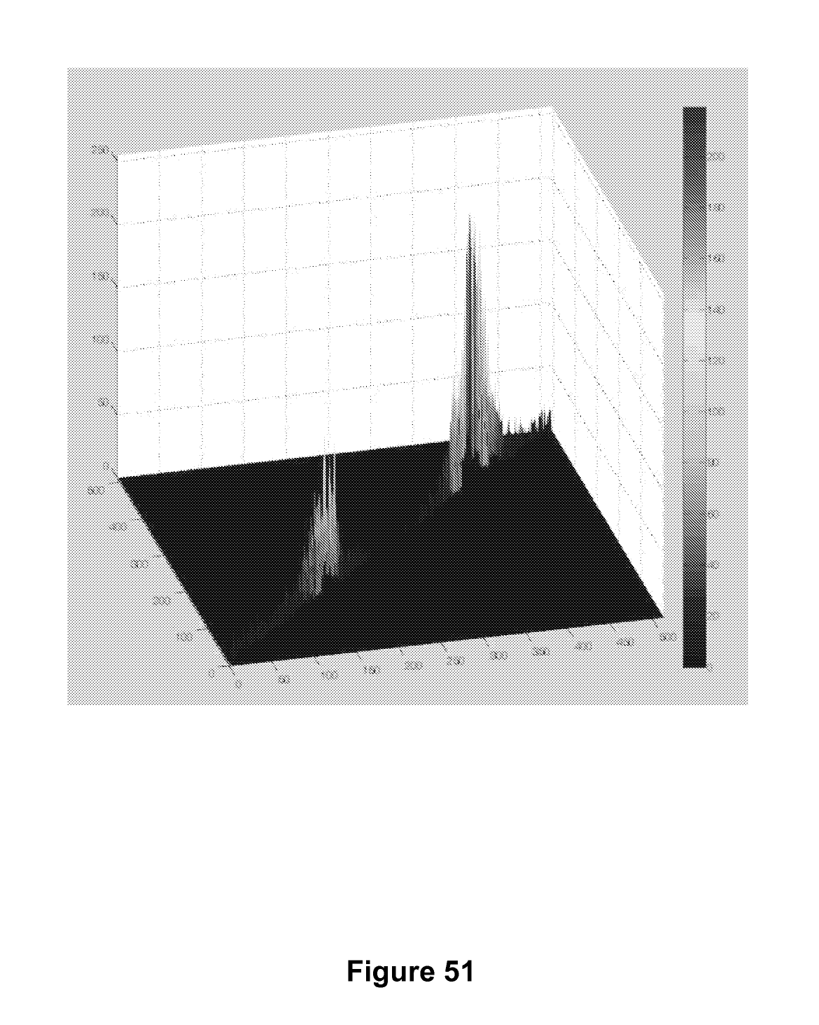

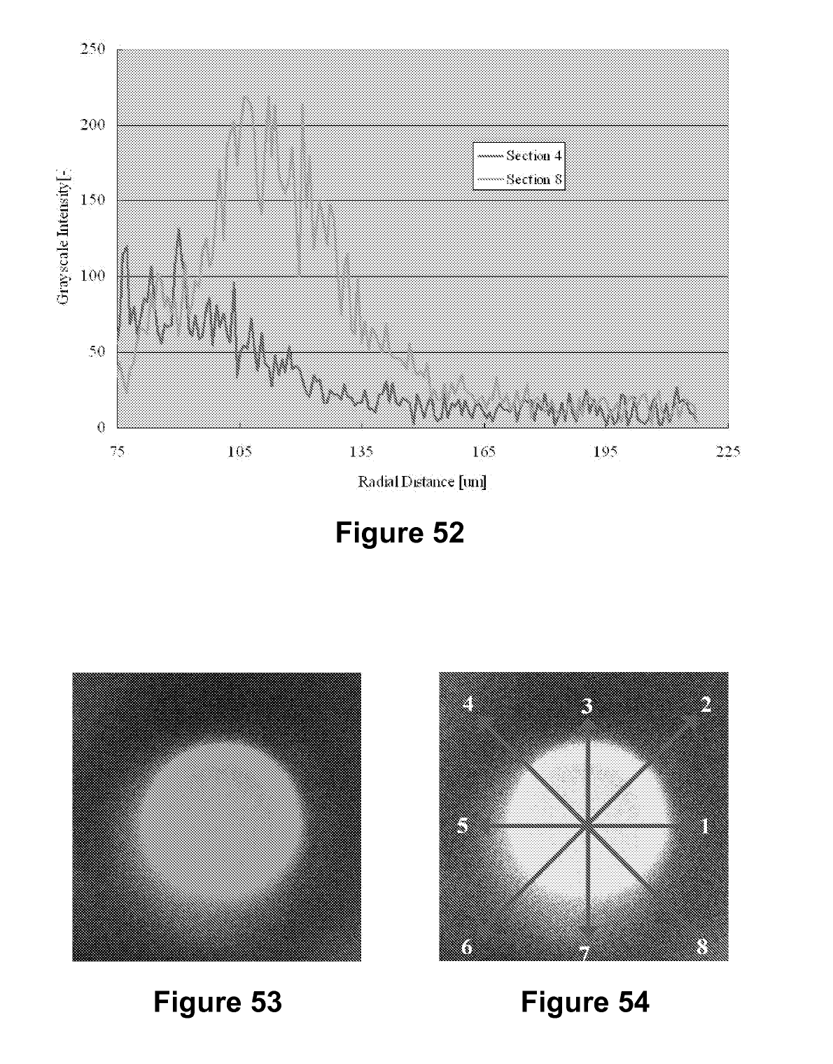

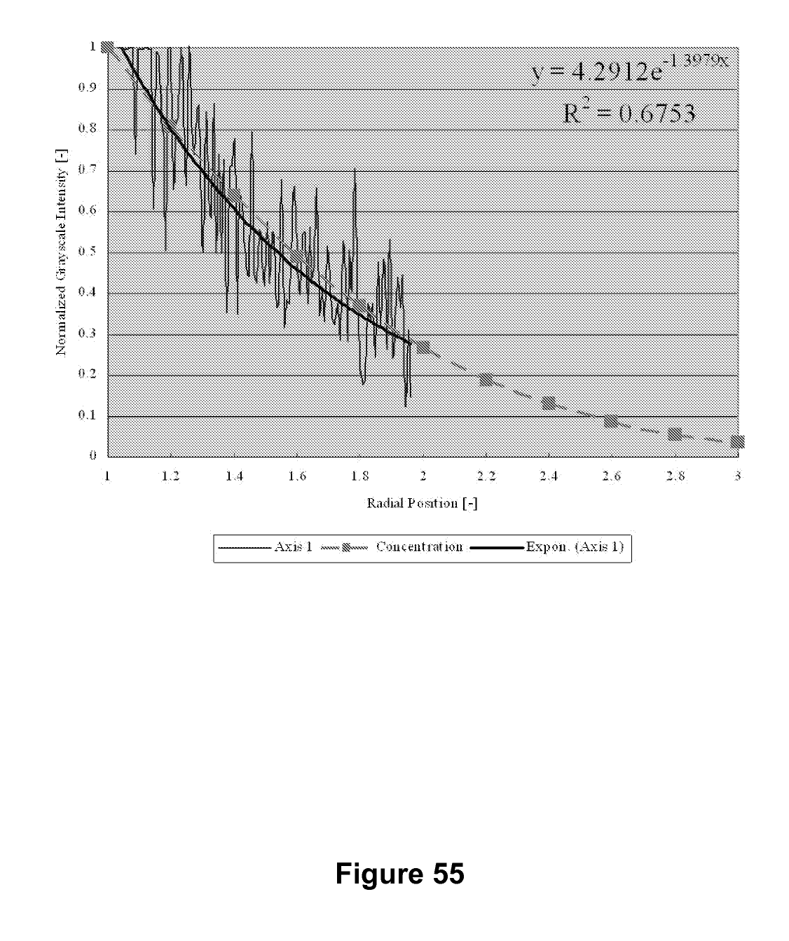

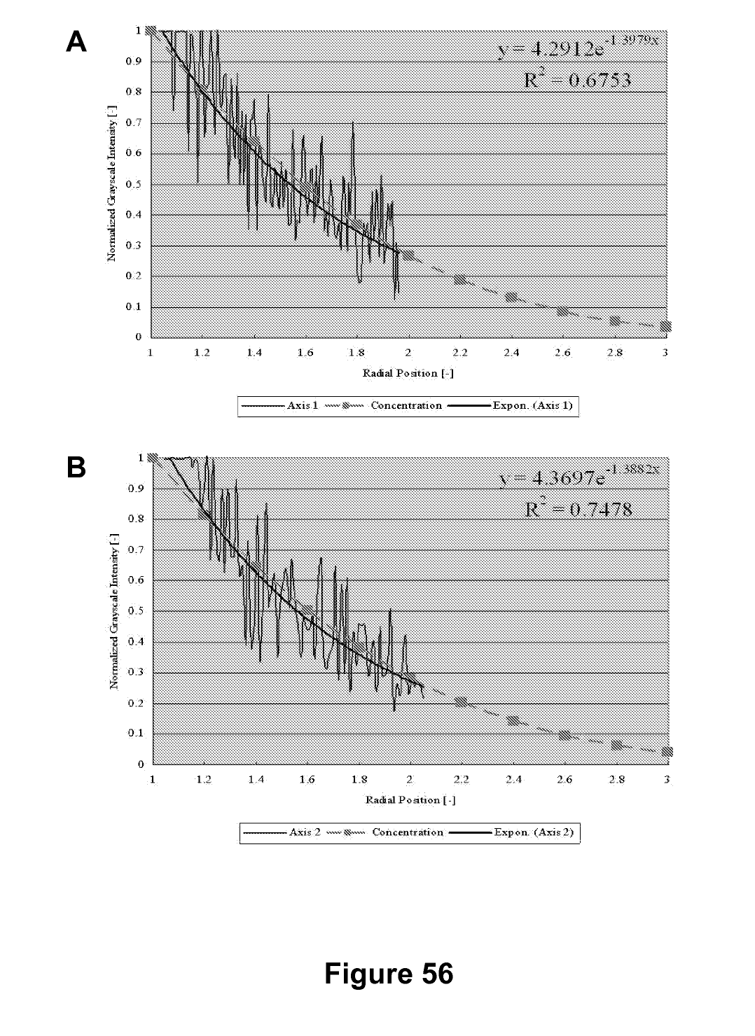

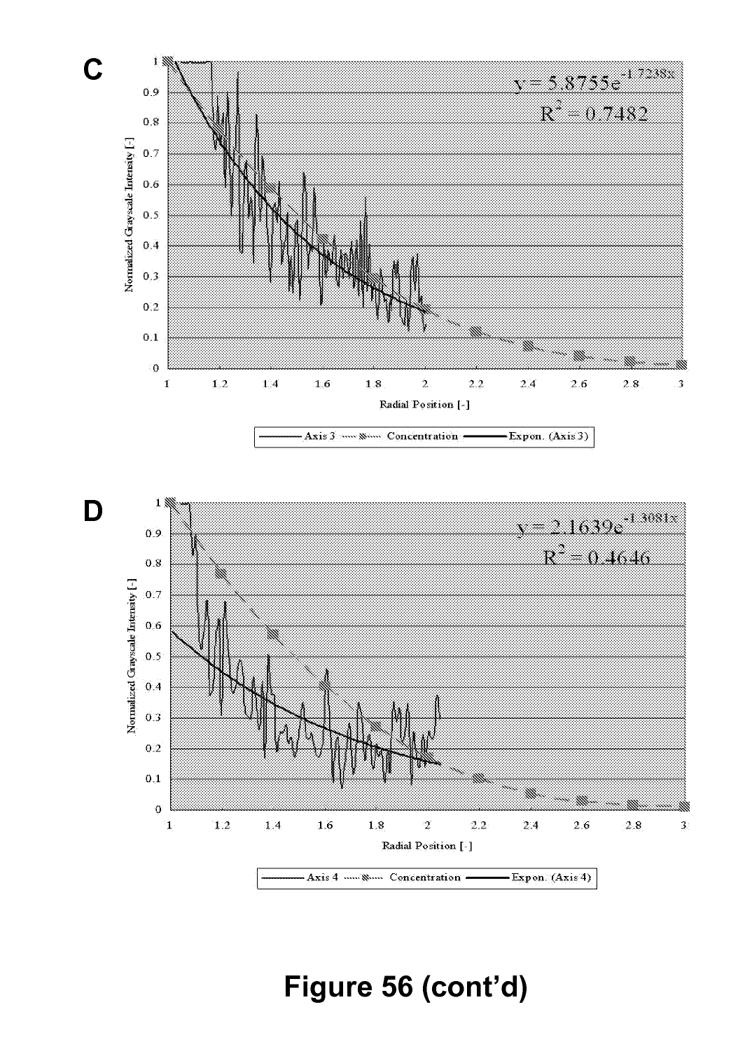

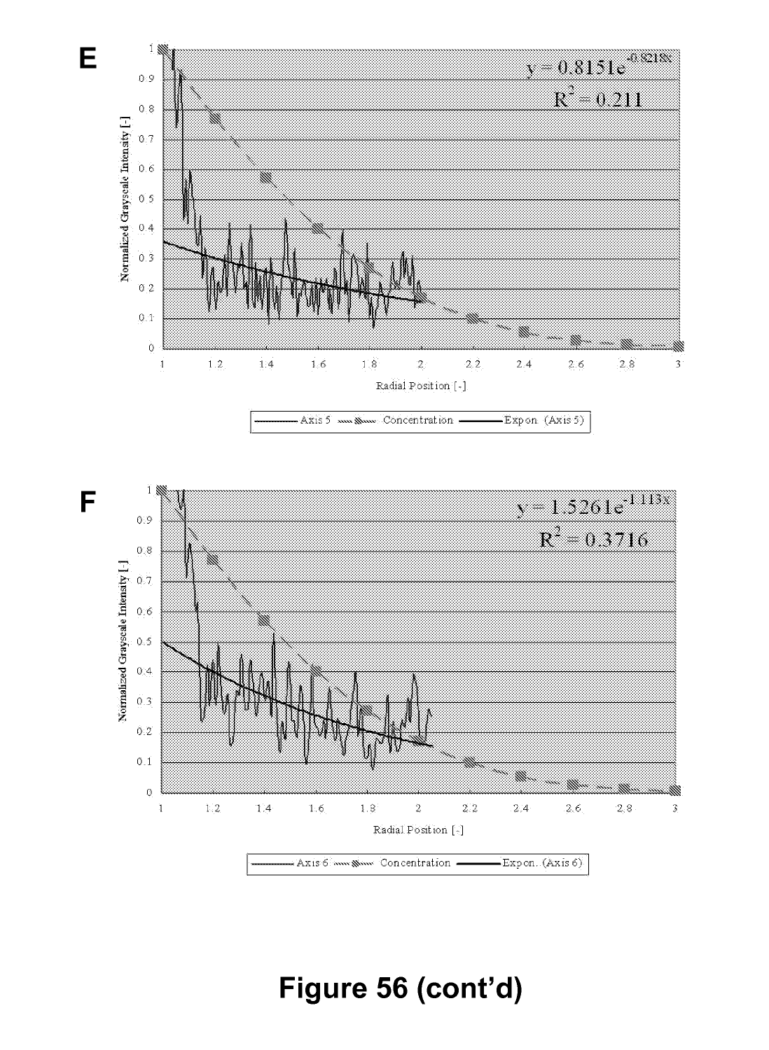

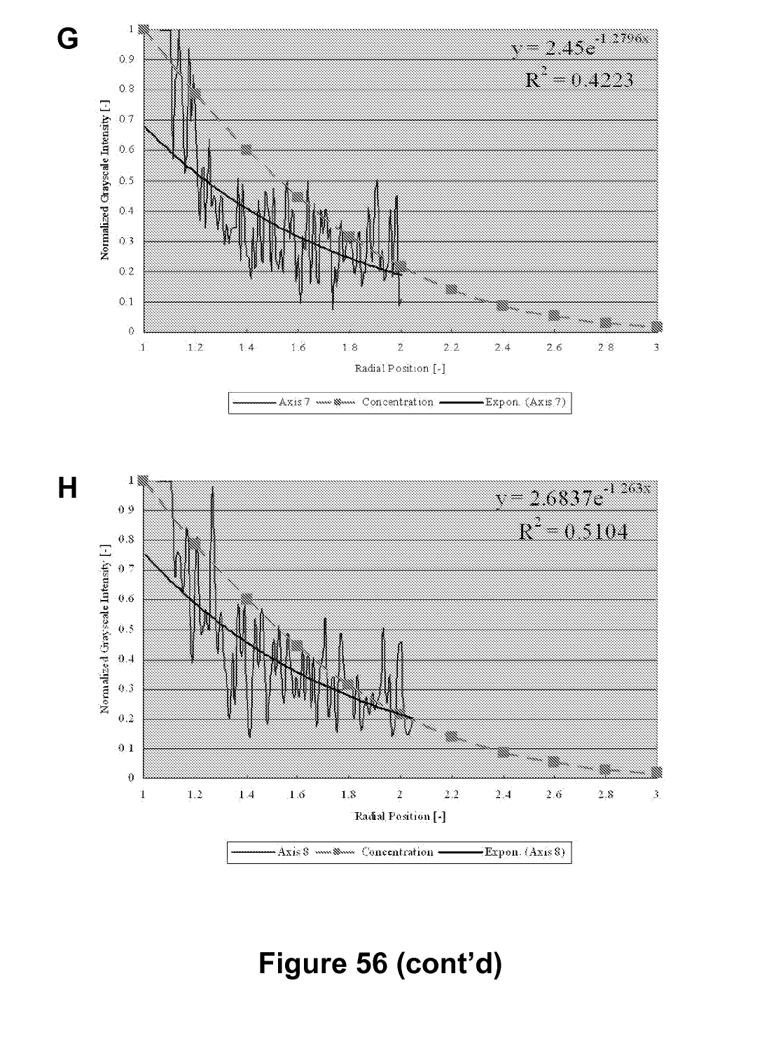

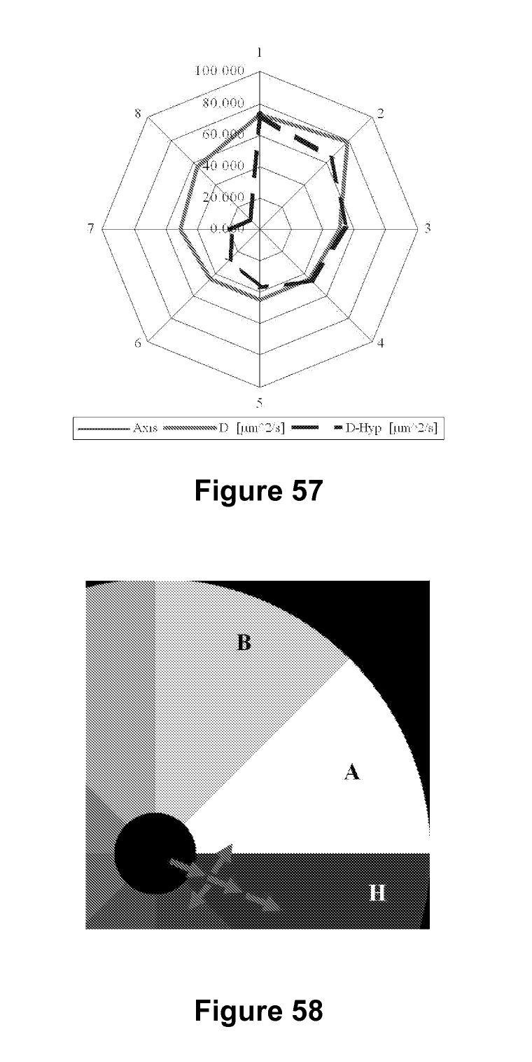

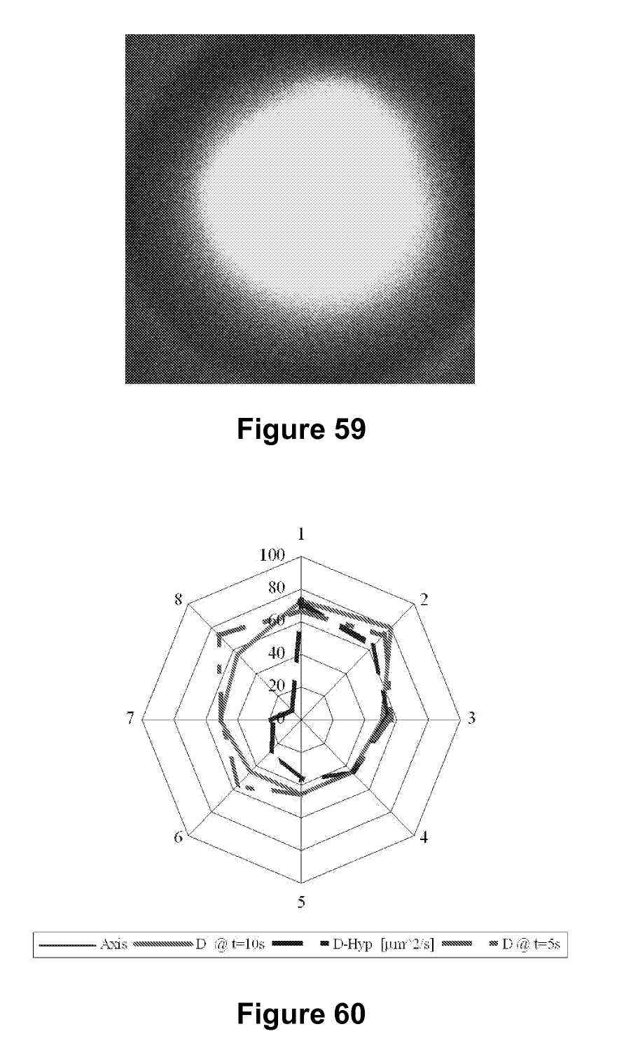

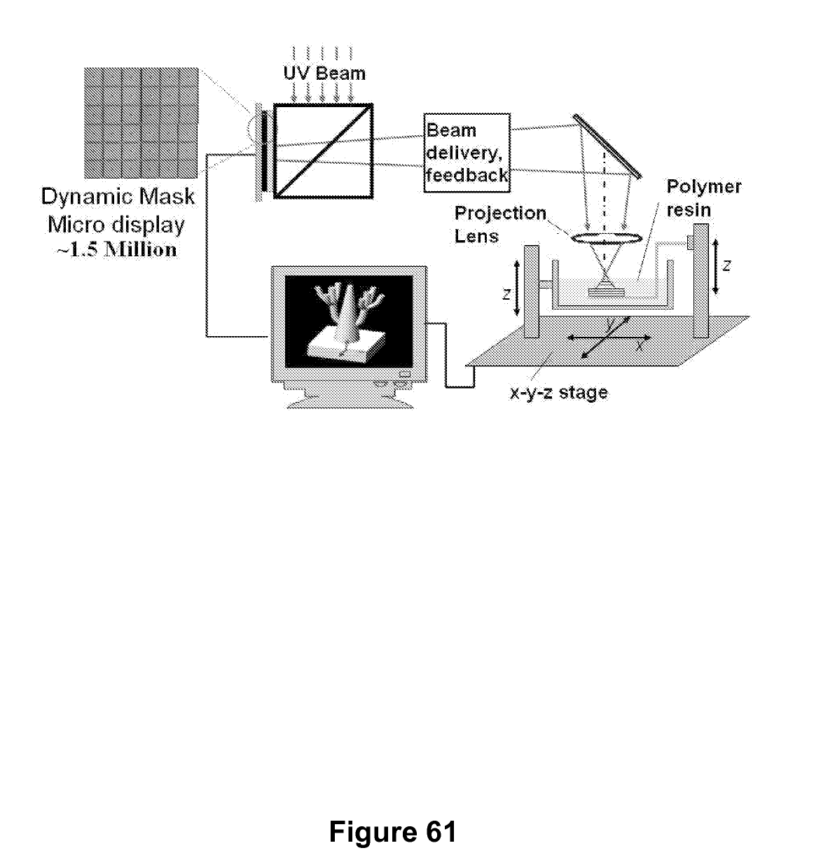

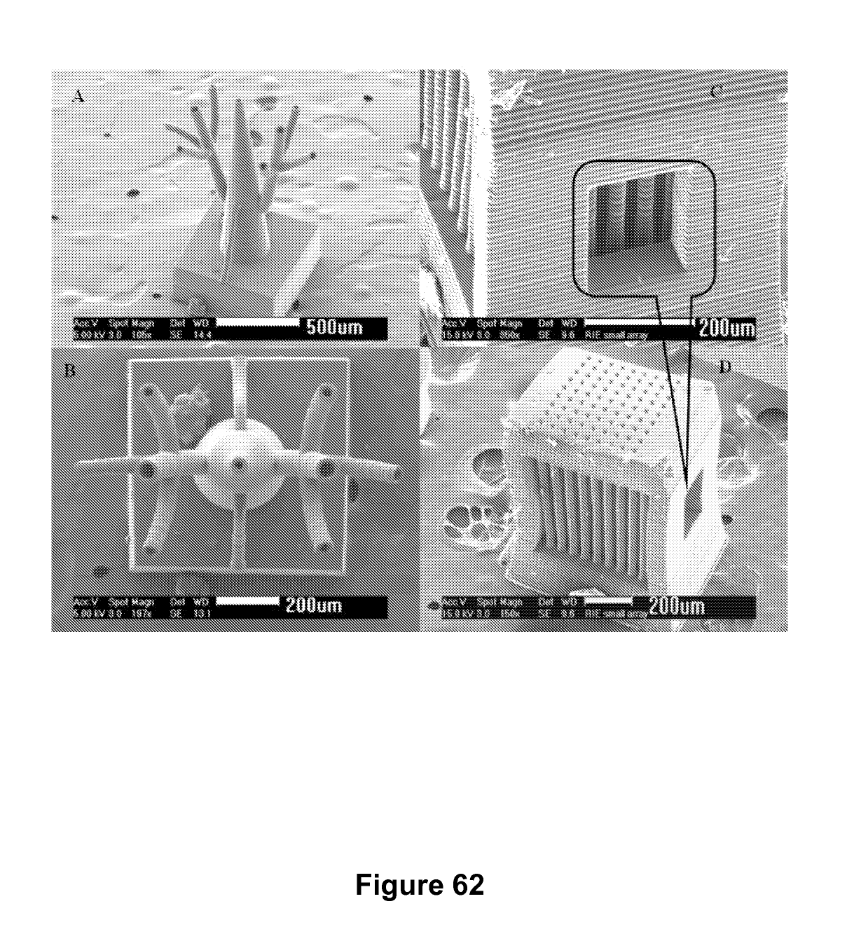

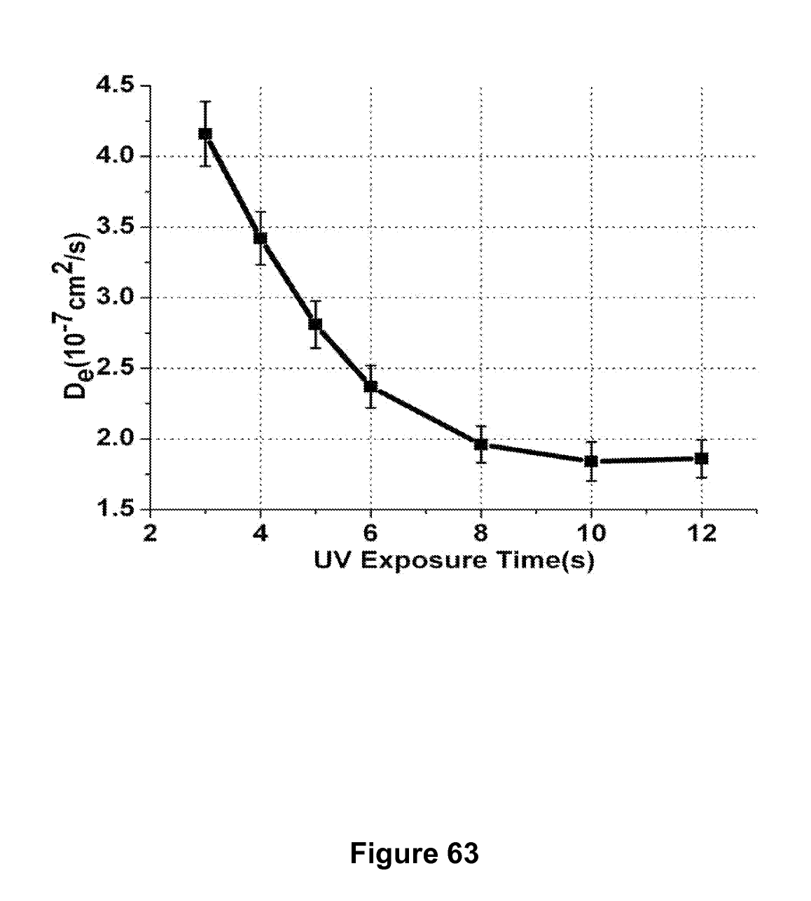

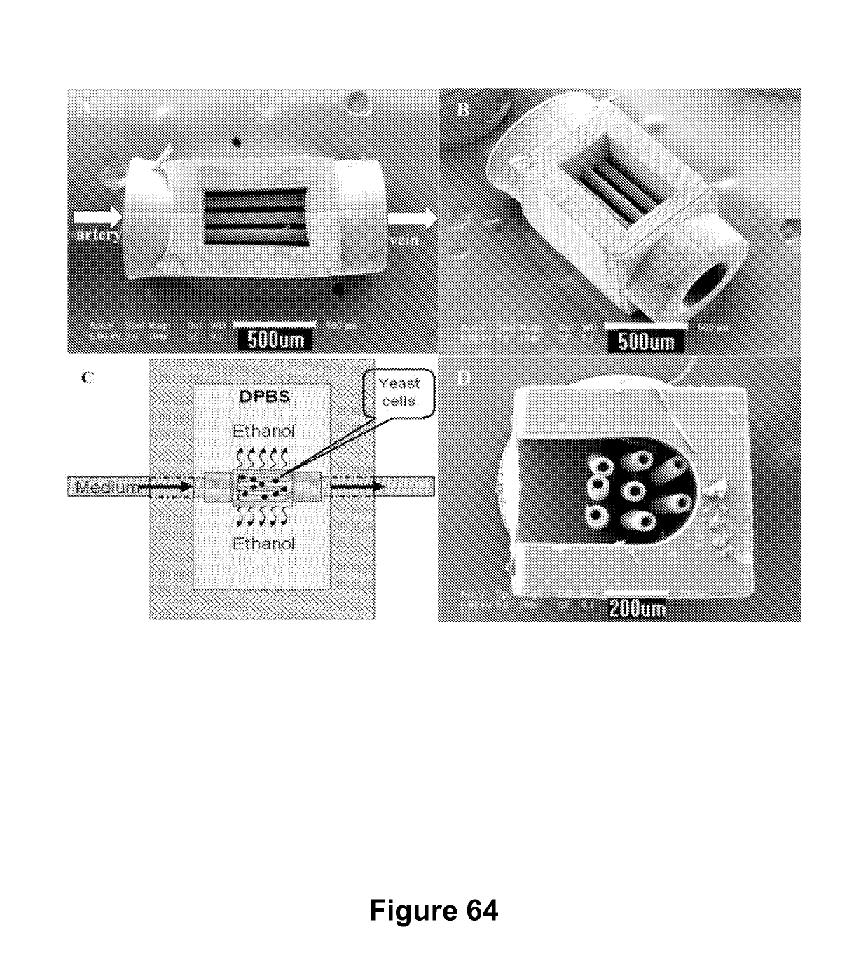

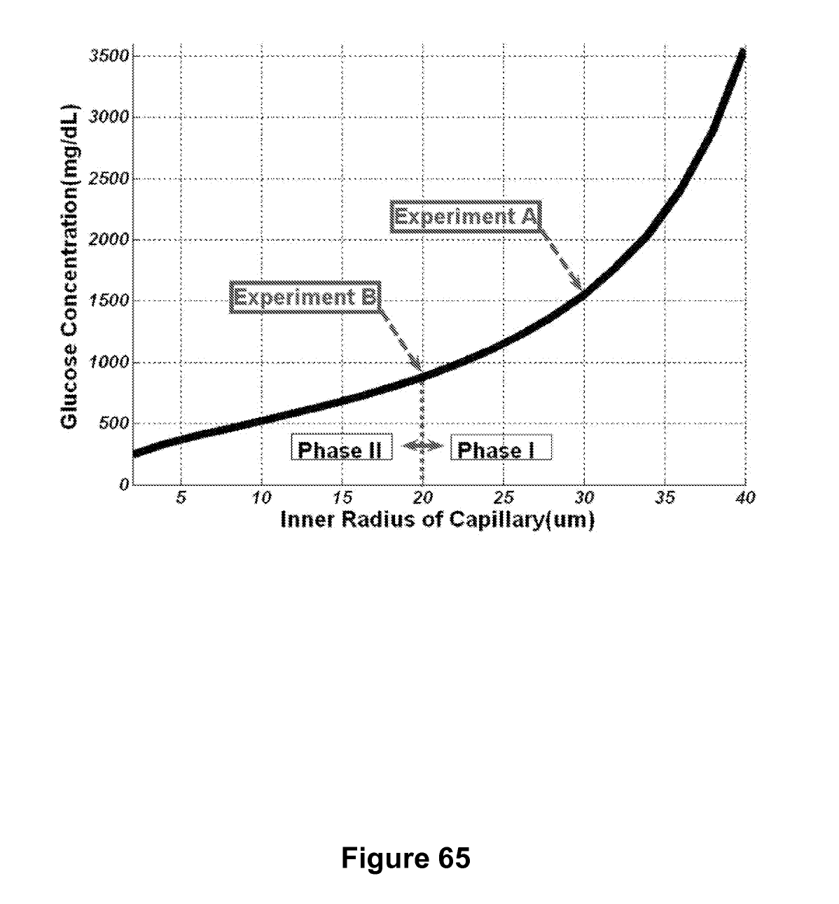

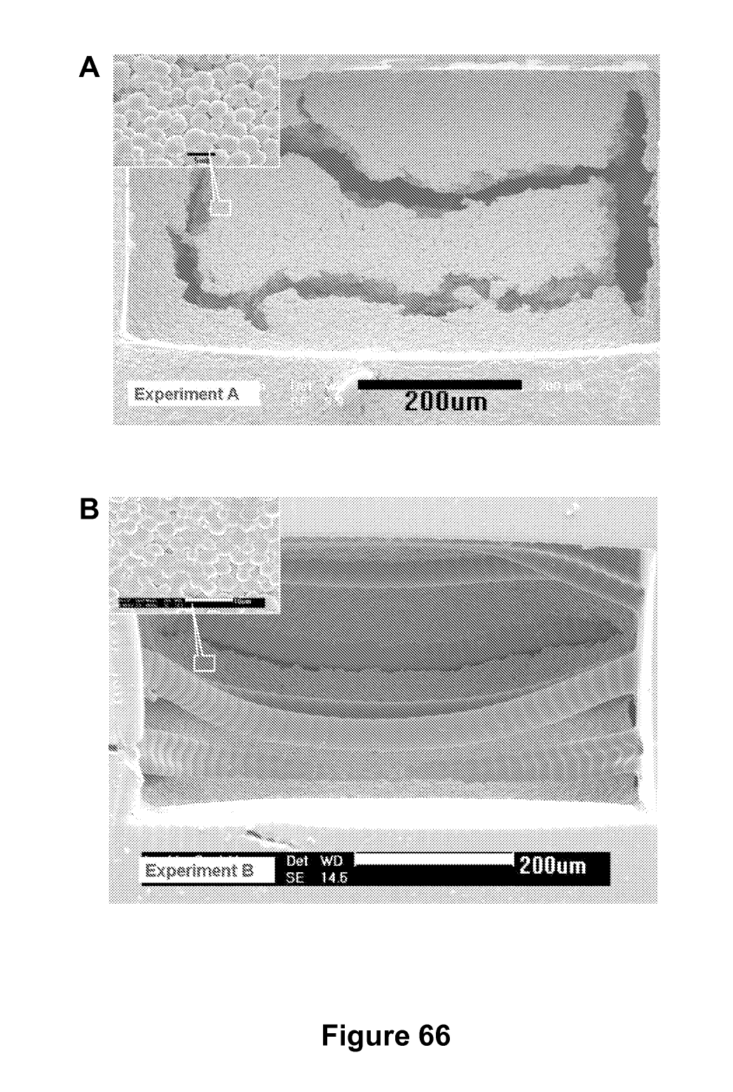

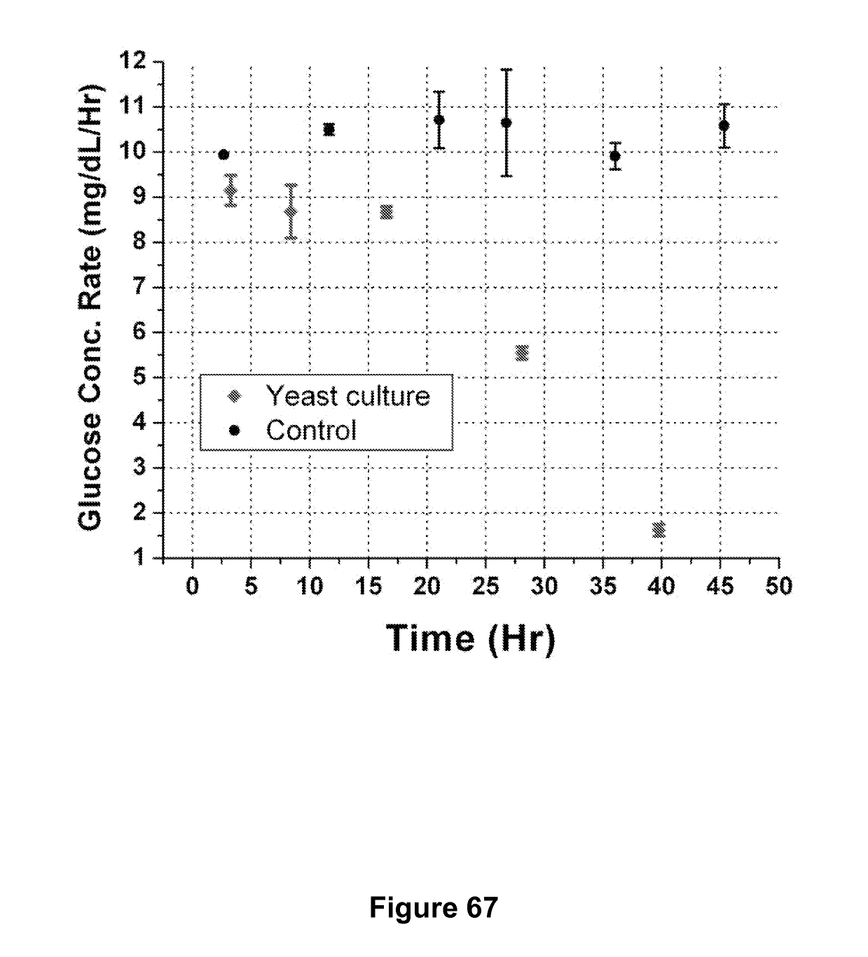

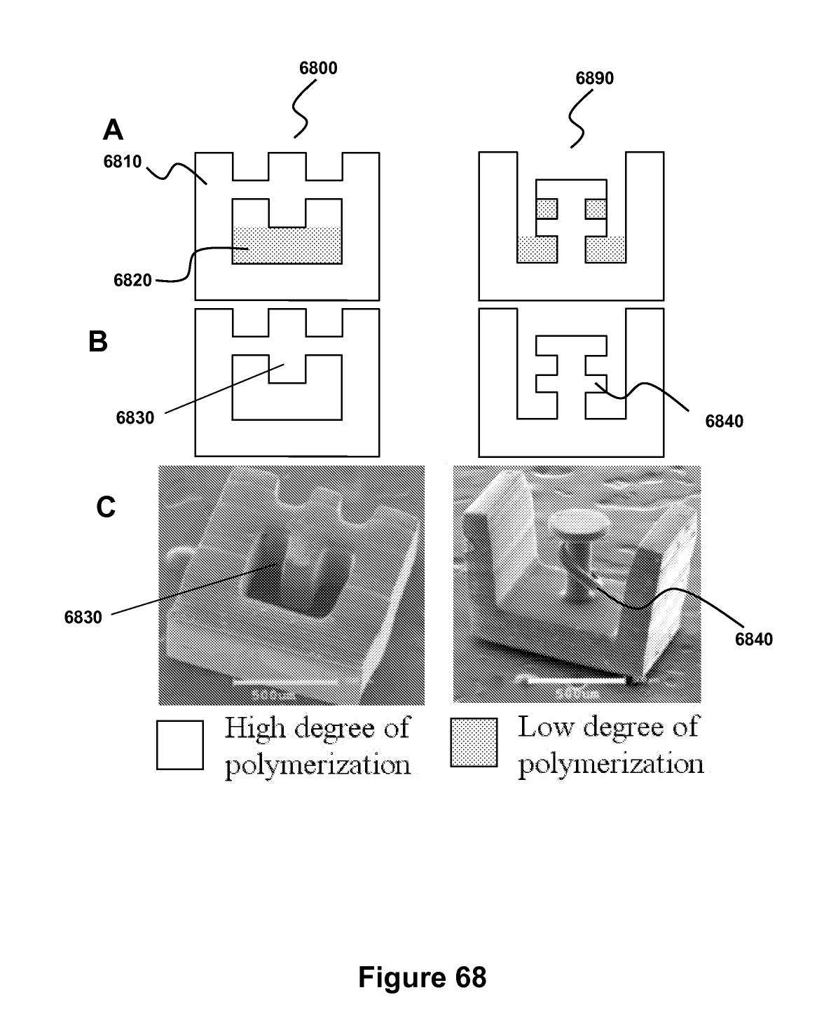

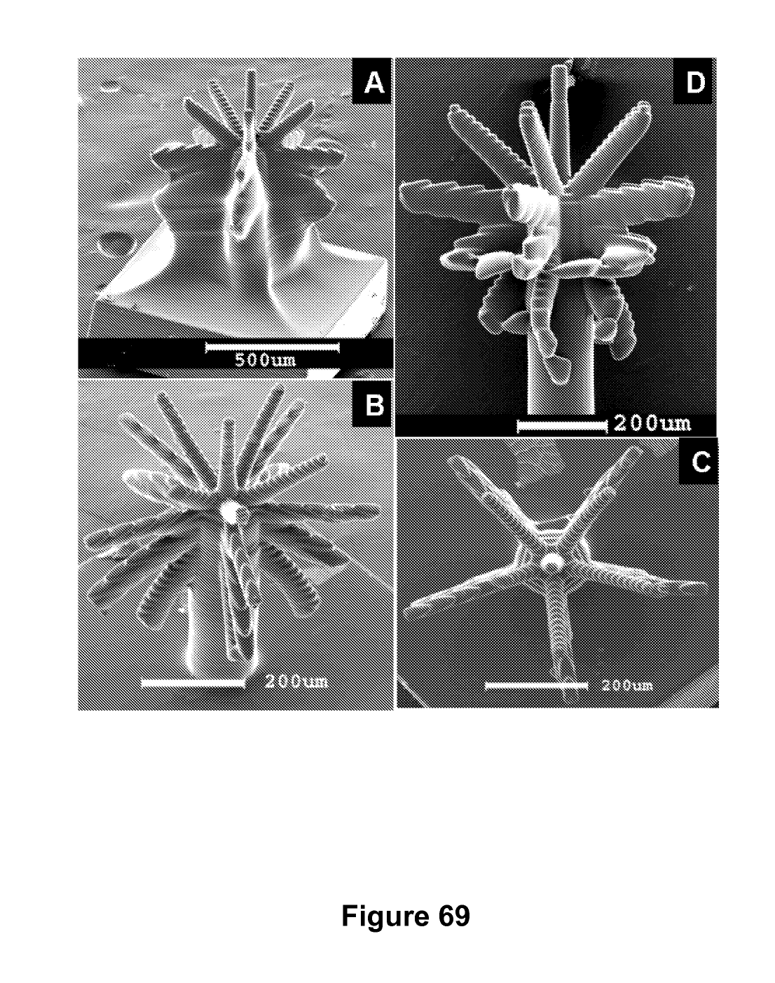

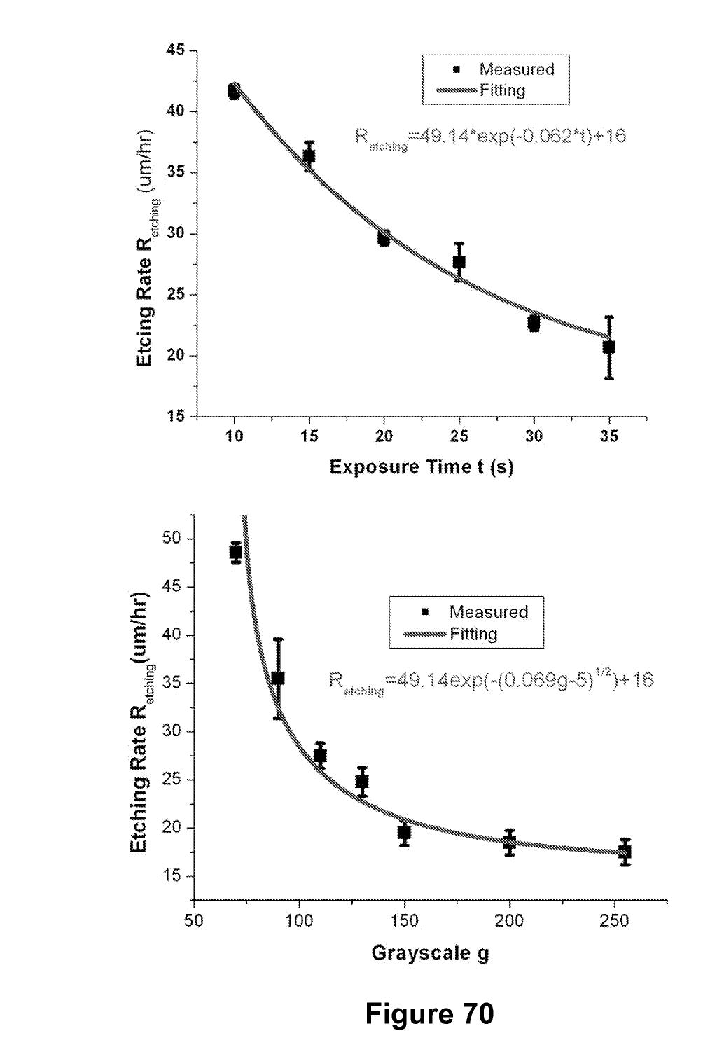

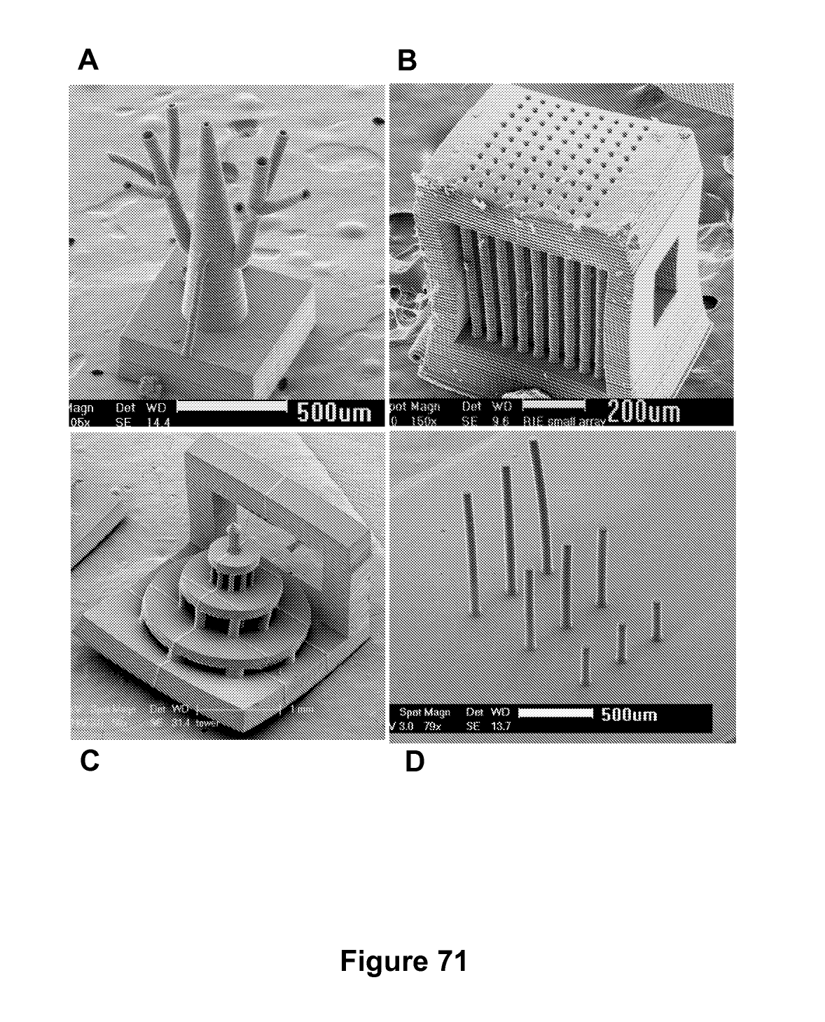

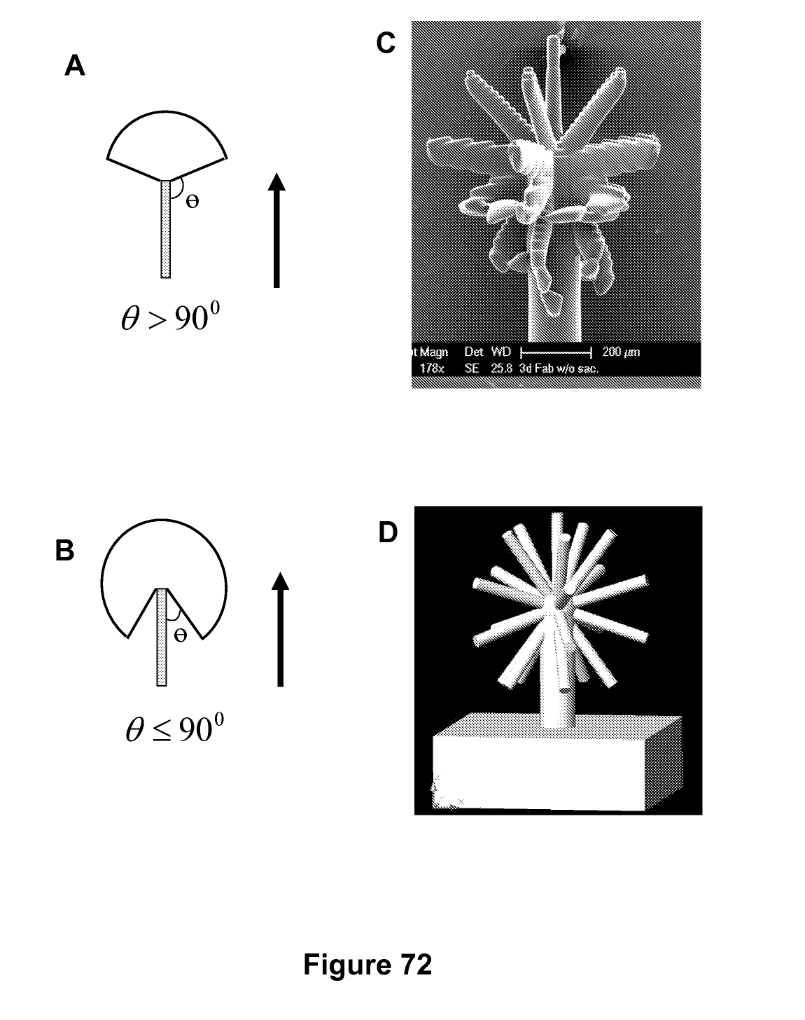

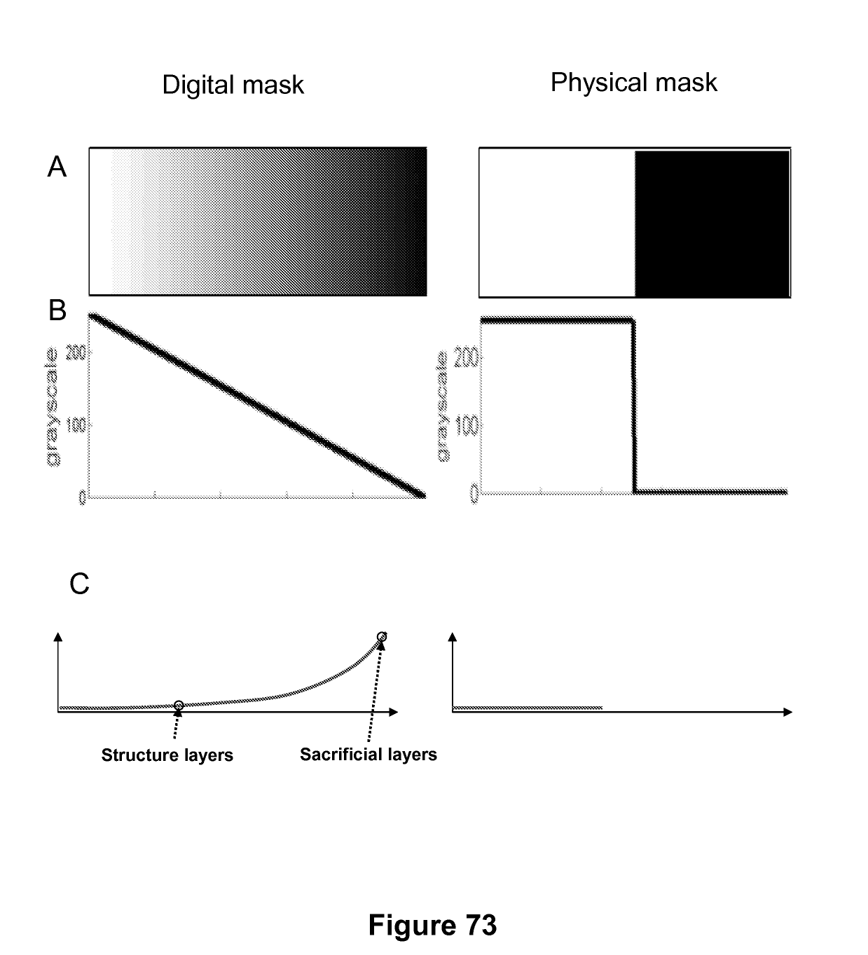

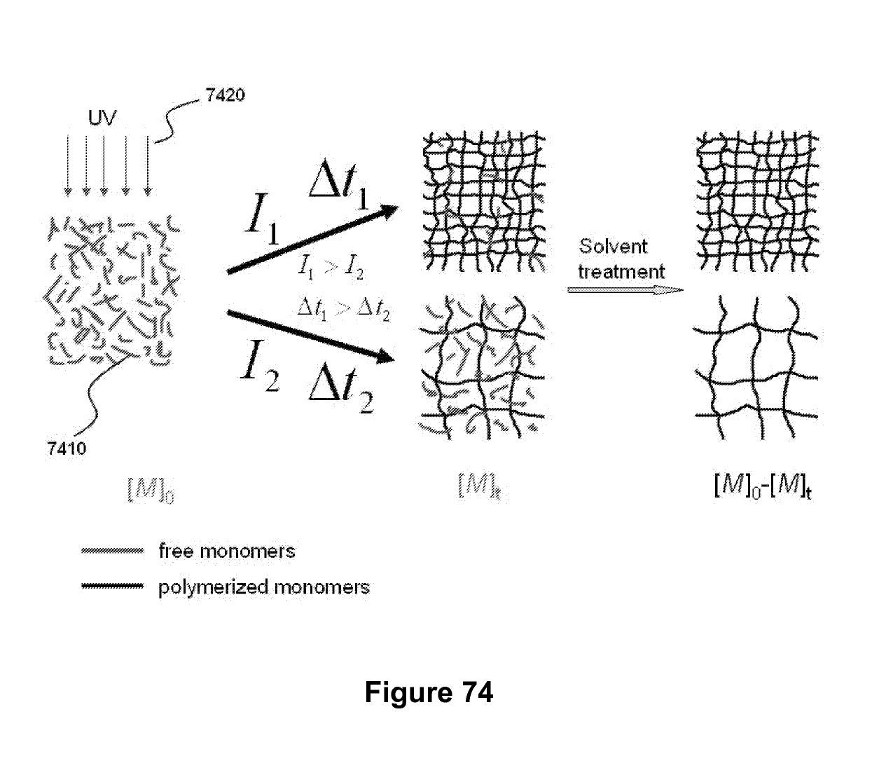

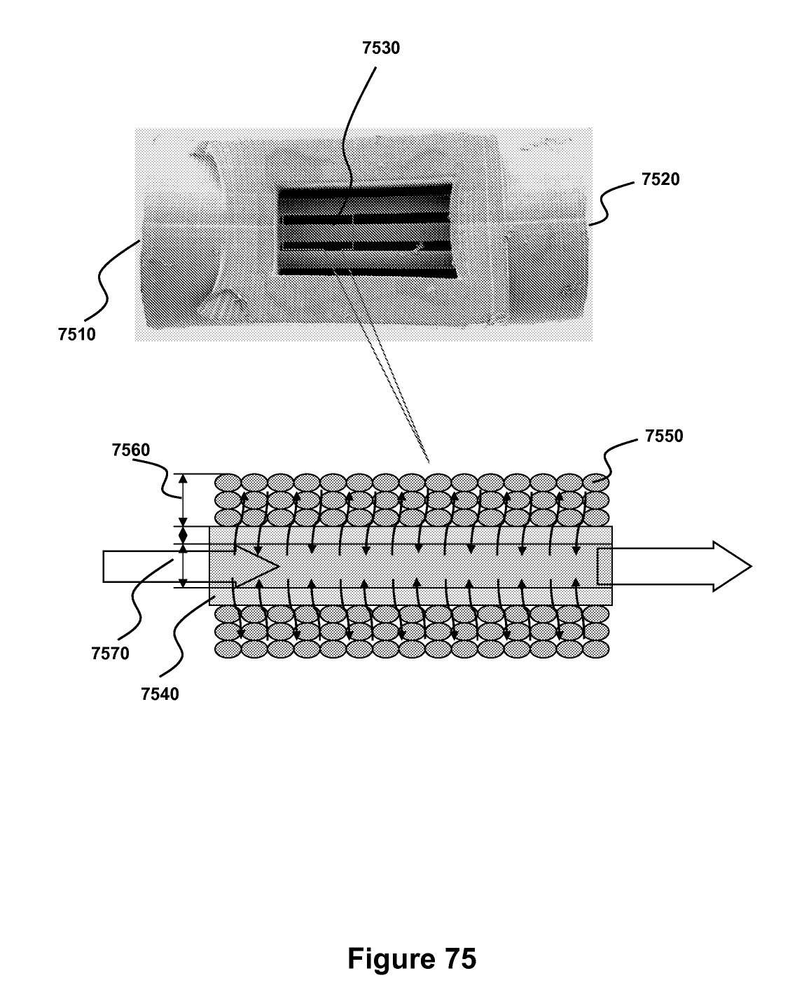

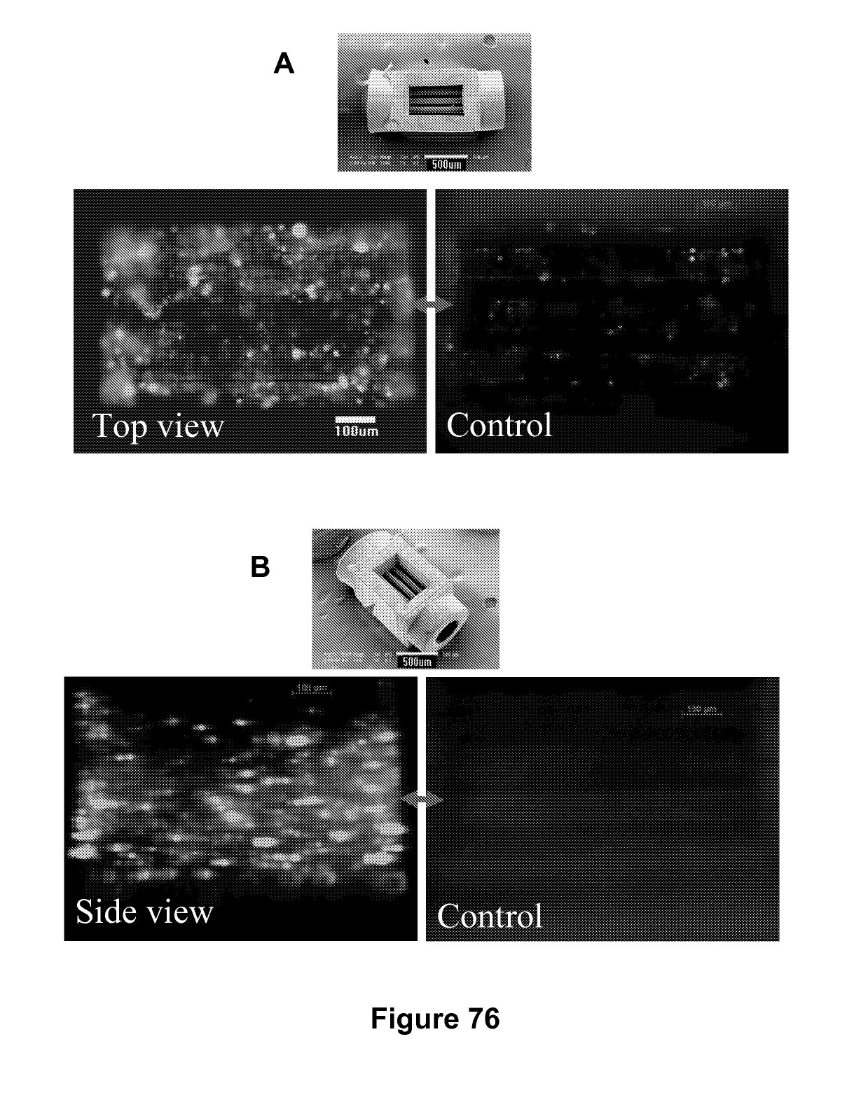

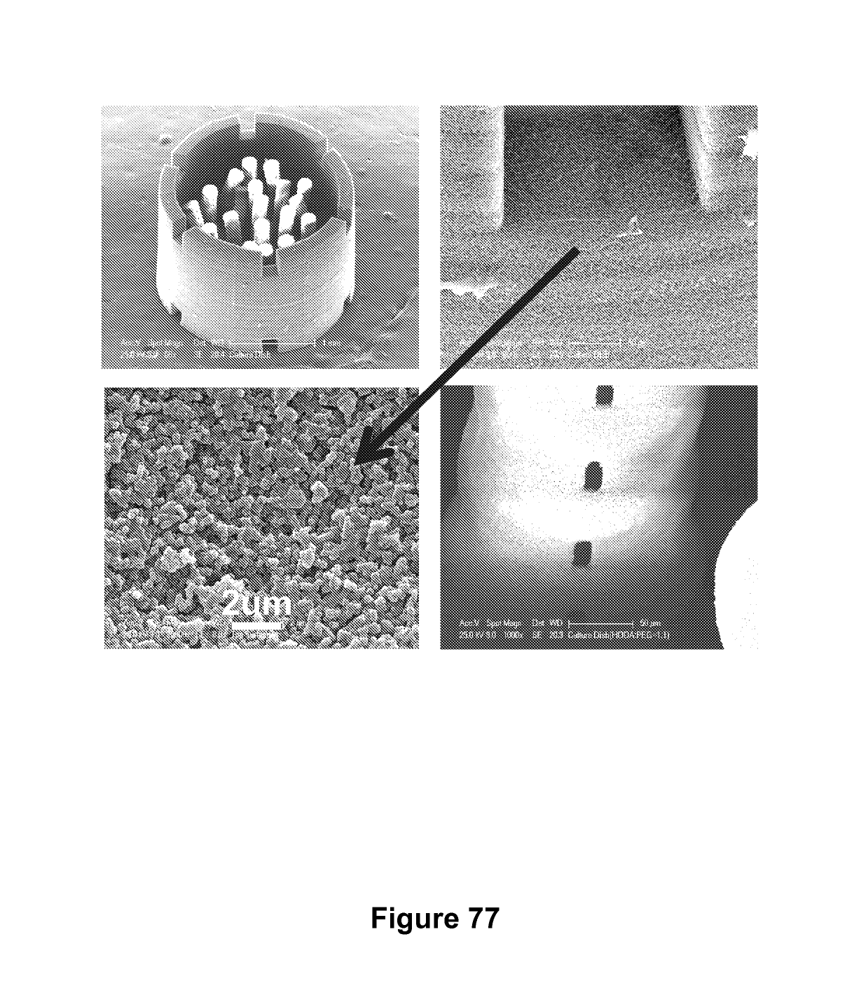

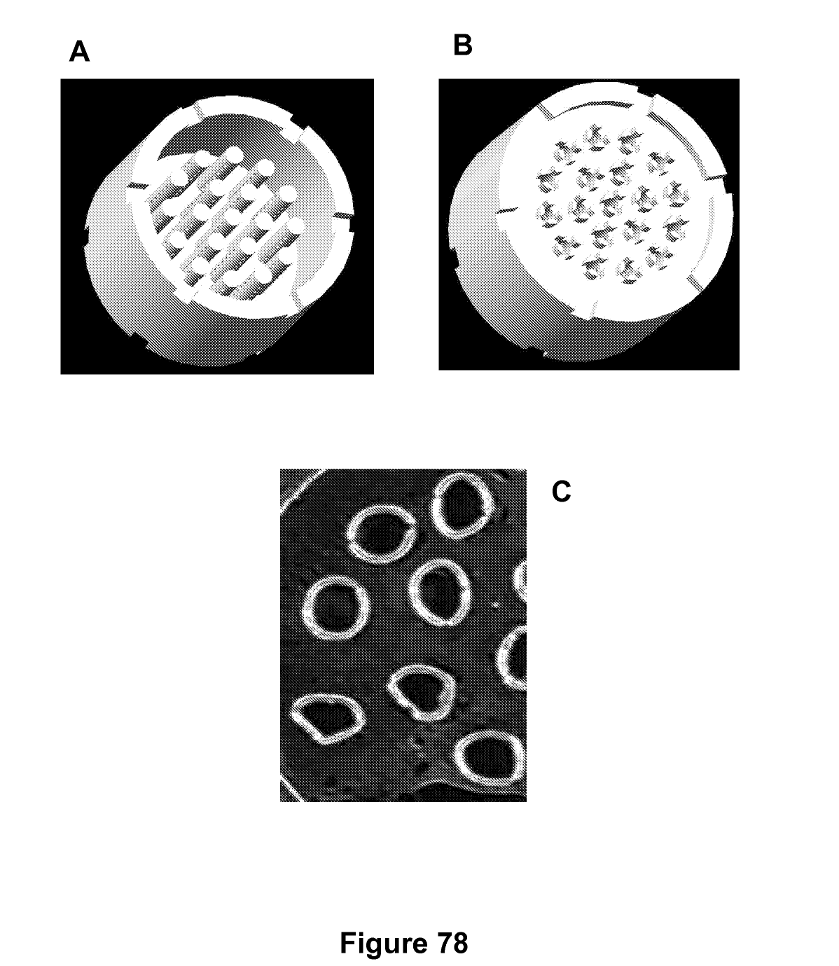

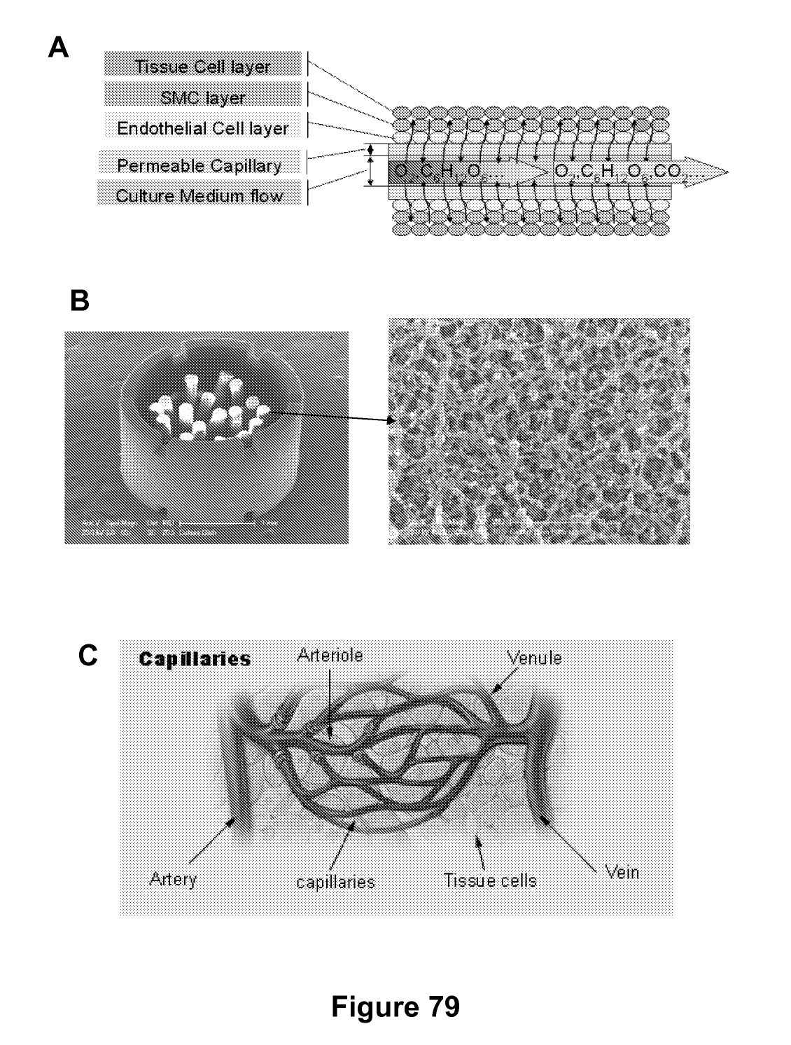

Penetration Depth: From the derivation above it is evident that the penetration depth of the monomer is an important factor in the generation of an accurate grayscale structure. The penetration depth is an empirical quantity that must be measured for each monomer solution at a particular wavelength. It is heavily influenced by the concentration of absorber in the monomer solution. The parameter may be determined by measuring the ratio of transmitted to incident light intensity through a sample of known thickness. Returning to the Beer-Lambert law, the penetration depth is given by:

$${D}_{p} ( λ ) = \frac{t}{ln ( \frac{{E}_{trans}}{{E}_{in}} )} \qquad ( 23 )$$

where t is the thickness of the sample, and Eₜᵣₐₙₛ/Eᵢₙ is the light transmittance ratio at a given wavelength. A Zeiss Axiovert 135 Inverted Research is used to measure the transmittance ratio of numerous solutions of PEGDA with different compositions. The microscope employs a 100 W mercury halogen lamp and a detector spanning wavelengths from 400-900 nm. The experimental setup is shown in FIG. 44.

Spacers of 15 μm are used to constrain the fluid thickness. The transmission ratios at a wavelength of 435 nm are recorded from the transmission plots generated by the Spec32 software running on an adjacent PC. Table 10 displays the measured transmission ratios and the calculated penetration depths of various PEG solutions.

Diffusion in Tunable PEGDA Structures: The diffusion properties of PEGDA-575 are examined to demonstrate that a PμSL system is capable of creating biological models whose diffusion properties can be tuned by the grayscale lithography method described herein.

Two Dimensional Pinwheel Design: Matlab code is used to fabricate the 2D pinwheel shown in FIG. 45. The eight equal-area regions of the pinwheel are each assigned a color value corresponding to ⅛ of the logarithmic grayscale intensity with region A at the maximum intensity and region H at the minimum. The grayscale values used for the regions are calculated for a layer thickness of 80 μm. By using it as a dynamic mask for a layer thickness of 10 μm, a structure of uniform height is fabricated with eight regions of varying light intensity and therefore having different crosslinking density. The intensity of the light transmitted through the darkest region (e.g., region H) remains sufficient to polymerize the entire 10 μm layer.

The fabricated pinwheel is 1000 μm in diameter. The dye injection hole in the center of the samples is 150 μm in diameter. The pinwheel forms one layer of a multilayer cylinder shown in FIG. 46. The additional cylinder layers are required to protect the pinwheel during transport and assure that the dye enters the pinwheel layer only in the radial direction and not from the top or bottom surfaces.

Measurement of Fluorescent Dye Diffusion: By placing water soluble dye in the center hole of the pinwheel, the diffusion rates of the pinwheel regions may be monitored simultaneously. The dye is Oregon Green® 488 BAPTA-2, an octopotassium salt (MW 1600) diluted in water, which is designed to fluoresce when bombarded with light at a wavelength of 488 nm. The dye's position in the pinwheel is monitored over time, thereby providing a measure of each section's diffusivity, by monitoring fluorescence over time using a confocal microscope.

Confocal microscopes permit a user to observe a fluorescing sample with superior resolution by using a pinhole filter that rejects light originating outside of the pinhole. This allows for sensitive light intensity measurements to be made in a very narrow image plane (on the order of 500 nm). A Leica SP2 Visible Laser Confocal Microscope is used to measure the fluorescence of the 2D pinwheel over a period of minutes after the Oregon dye solution was placed on the surface of the pinwheel cylinder. An argon laser at 488 nm is used to excite the dye, and the emitted light is observed between 525 and 575 nm.

After being placed on the surface of the cylinder, the dye solution wicks down the center channel via capillary action. The dye diffuses radially into all the horizontal layers of PEGDA-575. Variations in the radial diffusion of the dye should be observable in the eight different section of the pinwheel layer. FIG. 47 shows the central injection hole illuminated at the beginning of the measurement; little diffusion into the surrounding polymer has occurred. FIG. 48 shows a close view of the central hole 100 seconds after dye placement in the injection hole; the notches clearly delineate the boundaries of the eight pinwheel regions. A series of images at five second intervals is captured.

Confocal Data Processing: The confocal images provide a qualitative snapshot into the diffusion behavior of the pinwheel regions, but quantitative data is required for determining their diffusion coefficients, and hence the extent of diffusion tunability for the various pinwheel sectors. Therefore, the color confocal images are converted to grayscale bitmaps which are then read into Matlab using the “imread” function. The result is a matrix of values from 0 to 255 that represents the grayscale intensity of each pixel in the original bitmap. This matrix can then be displayed as a surface plot in Matlab, shown in FIG. 49. The false colors represent the fluorescent grayscale intensity at each pixel.

Analysis of the radial diffusion properties requires the isolation of data along a single radial line. FIG. 50 shows the matrix axes used to isolate radial lines from each of the eight regions: major and minor diagonals plus the horizontal and vertical centerlines. Data along each of these axes is extracted from the original matrix. FIG. 51 shows the isometric plot of the matrix's major diagonal (axes four and eight from FIG. 50). FIG. 52 clearly shows the preferential dye diffusion along axis eight (e.g., low light intensity exposure) as opposed to axis four (e.g., higher light intensity exposure). The radial offset of the peak intensity of the two regions is explained by the slight offset between the center of the matrix and the center of the bitmap.

Cylindrical Diffusion Analysis: This mathematical treatment of cylindrical diffusion follows Crank [42]. Diffusion in a long cylinder as a function of r and t only is given by:

$$\frac{\partial C}{\partial t} = \frac{1}{r} \frac{\partial }{\partial r} ( r D \frac{\partial C}{\partial r} ) \qquad ( 24 )$$

which is the statement of Fick's Second Law for a transient cylindrical case. The dye diffusing radially through the cylinder is assumed to follow the general exponential decay function:

C=uexp(−Dα²t) (25)

which is a solution to Equation (24) as long as D is constant throughout the volume and u is a function of r only that satisfies:

$$\frac{{\partial }^{2} u}{\partial {r}^{2}} + \frac{1}{r} \frac{\partial u}{\partial r} + {α}^{2} u = 0 \qquad ( 26 )$$

which is Bessel's equation of zero order. In order to determine the diffusion coefficients for the various sectors of the pinwheel, it is further assumed that: (1) The initial concentration of dye on the injection hole surface where r=a, Cₒ, is constant over the time elapsed; (2) The cylinder is of inner radius a=75 μm, outer radius B=300 μm, and a r B; (3) The initial dye concentration in the cylinder is zero throughout the volume; and (4) The concentration of dye at r=B is maintained at zero.

Under these conditions the solution for the radial distribution of dye concentration in the cylinder over time that satisfies the above equations is given by Crank [42: p84, Eq 5.62]:

$$\frac{C ( r )}{{C}_{0}} = \frac{Ln ( B / r )}{Ln ( B / a )} - π * {\sum }_{n = 1}^{∞} \frac{- {J}_{0} ( a {α}_{n} ) {J}_{0} ( B {α}_{n} ) {U}_{0} ( r {α}_{n} ) exp ( - D {α}_{n}^{2} t )}{{{J}_{0} ( a {α}_{n} )}^{2} - {{J}_{0} ( B {α}_{n} )}^{2}} \qquad ( 27 )$$

and:

U₀(rαₙ)=J₀(rαₙ)Y₀(Bαₙ)−J₀(Bαₙ)Y₀(rαₙ) (28)

where: C(r) is the concentration of dye at position r=a; C₀ is the initial concentration of dye in the injection hole; D is the dimensionless diffusion coefficient on the axis in question; J₀ is the Bessel function of the first kind zero order; and Y₀ is the Bessel function of the second kind zero order.

Additionally, αₙ is the n^{th} solution to:

U₀(aα)=J₀(α)Y₀(kα)−Y₀(α)J₀(kα)=0 (29)

Where k=B/a, the ratio of the outer to inner radii. The first n^{th} αₙ of Uₒ may be determined using the first four terms of this approximation given by McMahon, as reported by Grey and Matthews [43: Appendix III, vi]:

$${α}_{n} = δ + \frac{p}{δ} + \frac{q - {p}^{2}}{{δ}^{3}} \frac{r - 4 pq + 2 {p}^{3}}{{δ}^{5}} + … δ = \frac{n π}{k - 1} p = \frac{- 1}{8 k} q = \frac{100 ( {k}^{3} - 1 )}{3 {( 8 k )}^{3} ( k - 1 )} r = \frac{34336 ( {k}^{5} - 1 )}{5 {( 8 k )}^{5} ( k - 1 )} \qquad ( 30 )$$

The first five roots of Uₒ are given by Carslaw & Jaeger [44: Appendix IV, Table IV] for various values of k in Table 11. They show exact agreement with the McMahon approximation except where k is high and n is low. The k ratio for the cylinder in question is 6.67. Unfortunately the α₁ term, which makes the most significant contribution to the exponential decay, is the most affected by the approximation error. Therefore, given that the intensity data collected form the confocal images is limited to a region 1≦k≦3, the αₙ values for k=4 are acceptable for use in all calculations.

Cylindrical Diffusion Analysis: By further assuming that the intensity of the fluorescent dye is an accurate indicator of the dye concentration within the cylinder volume, the experimental data taken with the confocal microscope may be non-dimensionally compared with the mathematical solution described above. Further, the diffusion constant along a given axis of experimental data may be approximated by fitting the solution above to the data with an appropriate value of D.

FIG. 53 shows the raw fluorescent image at t=10 s, which is the last time for this data set where the dye filled the entire central volume of the injection hole, and therefore the last time when a constant concentration at r=a can be assumed. FIG. 54 shows the processed grayscale image of FIG. 53 with the matrix axes superimposed. FIG. 55 show the normalized grayscale intensity data (blue) plotted against the normalized radial position for axis 1. Thus, x=1 on FIG. 55 corresponds to r=75 μm at r=a, and x=3 corresponds to r=225 μm within the cylinder volume. The solid black lines represent a best-fit exponential trendline for the data in question; their equations and R² coefficients are given on the figures. The dashed pink line is the concentration solution given by Equation. (29) plotted over the normalized radial position for an optimized value of D.

FIG. 56A-H shows a similar best-fit diffusion coefficient along each of the eight axes at t=10 s. For consistency's sake the D coefficient for each axis is chosen so that the dashed pink line will intersect the end of the black solid line at x=2. The offset between the bitmap and matrix centers present in FIG. 52 was adjusted before the data was analyzed. The theoretical curve from Equation (27) produced excellent agreement with the observed data on axes 1, 2, & 3, good agreement on axis 8, poor agreement on axes 4 & 7, and very poor agreement on axes 5 & 6.

The values for the diffusion coefficients along the eight axes are given in Table 12, and they appear plotted in polar coordinates as the pink (solid) line in FIG. 58. The dashed line represents a hypothetical condition where the ⅛ intensity steps in the 2D pinwheel produce a correlated distribution of diffusion coefficients. The empirical data fails to show the large diffusion coefficient variance that should be present between regions A and H (the regions of maximum and minimum intensity, respectively).

Two factors are likely responsible for the lack of agreement between the theory presented and the empirical data recorded. First, the theory assumes radial diffusion only, but due to the pinwheel construction, there is a strong possibility that diffusion between regions is also taking place, especially in the region surrounding the central injection hole. For example, dye entering region H sees the highest diffusion coefficient in the sample. It diffuses isotropically as shown in FIG. 58 causing the dye concentration in region A, which should be the lowest in the sample, to increase significantly. Likewise, the high diffusion through region H may also cause an increase in the diffusion coefficient in region G, although the effect should not be as strong as in region A. Similarly, region G will affect region F in a similar manner, and region F will affect region E, and so on. Following this line of analysis it is reasonable to assume that axis 1 runs along the midline of region H and therefore: D₂>D₁ is a result of dye infiltration from region H into region G; and D₈ & D₇ are much higher than expected as a result of dye infiltration from region H into regions A & B.

Second, the radial diffusion theory assumes that the concentration at all points on the inner radius of the cylinder is equal and constant in time. Because of the sensitivity of the confocal microscope, once a dye concentration is reached that saturates the microscope's detector; any further increase in concentration will be undetectable. So, a confocal image of a fully saturated cross section may hide potentially significant concentration gradients. Therefore it is likely that, despite the saturation of the cross section visible in FIG. 54, the concentration on the surface of regions 4-7 was lower than the concentration on the surface of regions 3-8.

Further Analysis in Time: In order to examine the validity of the assumption of a constant D, the above analysis was repeated for t=5 s. The grayscale bitmap at t=5 sec is shown in FIG. 59 and the diffusion coefficients along each of the eight axes are shown in FIG. 60 and Table 13. FIG. 60 and Table 13 show that the diffusion coefficients along the eight axes are relatively constant when the two time periods are compared. The coefficients along the axes which have the best agreement with theory (axes 1, 2, & 3) show a standard deviation of 6% from the average.

In summary, accurately scaled models of biological tissues are fabricated from micro-CT scan data using PμSL. Furthermore, with grayscale lithography enhancement, PμSL is capable of controlled variation of the diffusion properties of a single PEGDA layer. Mathematical modeling of dye diffusion through the PEGDA-575 pinwheel indicates that its diffusion coefficients are tunable within 25% of the average value and are consistent over short periods of time. These results demonstrate the concept of tuning to accurately mimic the true diffusion properties of the biological system being modeled, such as variation in the diffusion coefficient for a blood vessel wall along the vascular tree.

### REFERENCES FOR EXAMPLES 1-4

[1] P. F. Jacobs, Rapid Prototyping and Manufacturing., 1st ed. Dearborn, Mich.: Society of Manufacturing Engineers, 1992, pp. 434.
[2] 3D Systems. Customer successes. http://www.3dsystems.com/appsolutions/atwork_listing.asp
[3] 3D Systems. SLA 7000 system. http://www.3dsystems.com/products/sla/sla7000/index.asp
[4] C. Sun, N. Fang, D. M. Wu and X. Zhang, “Projection micro-stereolithography using digital micro-mirror dynamic mask,” Sensors and Actuators A, vol. 121, p. 113, 2005.
[5] Castle Island Co. Worldwide guide to rapid prototyping. http://home.att.net/˜castleisland/rp_int1.htm
[6] K. Ikuta and K. Hirowatari, “Real three dimensional micro fabrication using stereo lithography and metal molding,” 6th IEEE Workshop on Micro Electrical Mechanical Systems, 1993.
[7] T. Takagi and N. Nakajima, 4th International Symposium on Micro Machine and Human Science, 1993.
[8] K. Ikuta, K. Hirowatari and T. Ogata, “Electrostatic micro actuator fabricated by micro stereo lithography (1H process),” in Proceedings from the 5th International Symposium on Micro Machine and Human Science, pp. 135, 1994.
[9] S. Ballandras, M. Calin, S. Zissi, A. Bertsch, J. C. André and D. Hauden. Microstereophotolithography and shape memory alloy for the fabrication of miniaturized actuators. Sensors and Actuators A, 62, pp. 741-747, 1997.
[10] X. Zhang, X. N. Jiang and C. Sun, “Micro-stereolithography of polymeric and ceramic microstructures,” Sensors and Actuators A, vol. A7, pp. 149, 1999.
[11] K. Ikuta, K. Hirowatari and T. Ogata, “Three dimensional micro integrated fluid systems (MIFS) fabricated by stereo lithography,” in Proceedings from IEEE Workshop on Micro Electro Mechanical Systems, 1994, pp. 1-6, 1994.
[12] S. Maruo and K. Ikuta, “Three-dimensional microfabrication by use of single-photon-absorbed polymerization,” Appl. Phys. Lett., vol. 76, 2000.
[13] S. Maruo, O, Nakamura and S. Kawata, “Three-dimensional microfabrication with two-photon-absorbed photopolymerization,” Optics Letters, vol. 22, pp. 132, 1997.
[14] S. Maruo and S. Kawata, “Two-Photon-Absorbed Near-Infrared Photopoly-merization for Three-Dimensional Microfabrication,” J. MEMS, vol. 7, pp. 411, 1998.
[15] S. Maruo and K. Ikuta, “Movable microstructures made by two-photon three-dimensional microfabrication,” in Proceedings of 1999 International Symposium on Micromechatronics and Human Science, 1999, pp. 173-178.
[16] S. Kawata, H. B. Sun, T. Tanaka and K. Takada, “Finer features for functional microdevices,” Nature, vol. 412, pp. 697, 2001.
[17] I. Wang, M. Bouriau, P. L. Baldeck, C. Martineau and C. Andraud, “Three-dimensional microfabrication by two-photon-initiated polymerization with a low-cost microlaser,” Optics Letters, vol. 27, pp. 1348, 2002.
[18] A. Bertsch, J. Y. Jezequel and J. C. Andre, “Study of the spatial resolution of a new 3D microfabrication process” J of Photochem. & Photobiol., vol. A107, pp. 275, 1997.
[19] N. Fang, “Engineering Subwavelength Photonic Metastructures and Devices,” Ph.D. dissertation, University of California at Los Angeles Department of Mechanical Engineering, 2004.
[20] K. Ikuta, T. Ogata, M. Tsubio and S. Kojima, “Development of mass productive micro stereo lithography (mass-1H process),” in Proceedings from the Ninth Annual International Workshop on Micro Electro Mechanical Systems, 1996, pp. 301-306.
[21] D. Dendukuri, D. Pregibon, J. Collins, T. A. Hatton and P. S. Doyle, “Continuous-flow lithography for high-throughput microparticle synthesis,” Nature Materials, vol. 5, pp. 365-369, 2006.
[22] M. Cabrera, A. Bertsch, J. Chassaing, J. Y. Jezequel and J. C. Andre, “Microphotofabrication of very small objects: Pushing the limits of stereophotolithography,” Mol. Cryst. and Liq. Cryst., vol. 314-315, pp. 525, 1998.
[23] A. Bertsch, S. Jiguet, H. Hofmann and P. Renaud, “Ceramic microcomponents by microstereolithography,” in Proceedings on 17th IEEE International Conference on Micro Electro Mechanical Systems, 2004, pp. 728.
[24] C. Provin and S. Monneret, “Complex ceramic-polymer composite microparts made by microstereolithography,” IEEE Transactions on Electronics Packaging Manufacturing, vol. 25, pp. 59-63, 2002.
[25] E. Sachlos and J. T. Czernuszka, “Making Tissue Engineering Scaffolds Work: Review on the Application of Solid Freeform Fabrication Technology to the Production of Tissue Engineering Scaffolds,” European Cells & Materials, vol. 5, pp. 29-40, 2003.
[26] C. G. Xia, N. Fang, C. Sun, D. M. Wu and X. Zhang, “3D Microfabricated Bioreactors,” Proceedings from 2006 Nanotech Conference, pp. 140-143, 2006.
[27] Y. Lu, G. Mapili, G. Suhali, S. Chen and K. Roy, “A digital micro-mirror device-based system for the microfabrication of complex, spatially patterned tissue engineering scaffolds,” Journal of Biomedical Materials Research Part A, vol. 77A, pp. 396-405, 2006.
[28] P. G. Conrad II, P. T. Nishimura, D. Aherne, B. J. Schwartz, D. Wu, N. Fang, X. Zhang, M. J. Roberts and K. J. Shea, “Functional Molecularly Imprinted Polymer Microstructures Fabricated Using Microstereolithography,” Advanced Materials, vol. 15, pp. 1541-1544, 2003.
[29] I. K. Kwon and T. Matsuda, “Photo-polymerized microarchitectural constructs prepared by microstereolithography (μSL) using liquid acrylate-end-capped trimethylene carbonate-based prepolymers,” Biomaterials, vol. 26, pp. 1675-1684, 2005.
[30] K. Ikuta and M. Nokata, “Two-lead-wire drive for multi-micro actuators [medical micro robots],” in Proceedings of the IEEE International Conference on Robotics and Automation, pp. 2378-2384, vol. 3, 1999.
[31] K. Ikuta, “Biomedical micro device fabricated by micro stereo lithography (1H process)” in Proceedings of the Sixth International Symposium on Micro Machine and Human Science, pp. 67-70, 1995.
[32] K. Ikuta, “Biochemical IC using micro stereolithography-towards artificial cellular device,” in Proceedings from International Conference on Microprocesses and Nanotechnology, pp. 26-27, 2000.
[33] T. M. Binder, D. Moertl, G. Mundigler, G. Rehak, M. Franke, G. Delle-Karth, W. Mohl, H. Baumgartner and G. Maurer, “Stereolithographic biomodeling to create tangible hard copies of cardiac structures from echocardiographic data: In vitro and in vivo validation,” Journal of the American College of Cardiology, vol. 35, pp. 230-237, 2000/1.
[34] M. Poulsen, C. Lindsay, T. Sullivan and P. D'Urso, “Stereolithographic modelling as an aid to orbital brachytherapy,” International Journal of Radiation Oncology, Biology, & Physics, vol. 44, pp. 731-735, 1999/6/1.
[35] K. Ikuta and K. Hirowatari, “Real three dimensional micro fabrication using stereo lithography and metal molding,” in Proceedings of Micro Electro Mechanical Systems, pp. 42-47, 1993.
[36] J. H. Chen, C. A. Chen and W. H. Hsieh, “Electrowetting Technique for Lens Fabrication,” Proc. IEEE Intl. Conf. on Mechatronics, pp. 768-770, 2005.
[37] S. Kuiper and Hendriks, B. H. W., “Variable-focus liquid lens for miniature cameras,” Applied Physics Letters, vol. 85, pp. 1128-1130, 2004.
[38] J. Lienemann, A. Greiner and J. G. Korvink, IEEE Transactions on Computer-Aided Design of Integrated Circuits and Systems, vol. 25, pp. 234-247, 2006.
[39] Sartomer Company, Inc., http://www.sartomer.com/TechLit/4024.pdf
[40] N. V. SkyScan, “SkyScan 1172 Desktop X-Ray Microtomograph Manual,” 2005.
[41] D. Wu, “Microfabrication of three-dimensional structures and characterization of molecular machines,” Ph.D. Dissertation, Chapter 4, UCLA, 2005.
[42] J. Crank, Mathematics of Diffusion, Chapter 5, Oxford, UK: Clarendon Press, 1979.
[43] A. Gray and G. B. Matthews, A Treatise on Bessel Functions and their Application to Physics. Second ed. New York: Dover Publications, 1966, pp. 327.
[44] H. S. Carslaw and C. J. Jaeger, Conduction of Heat in Solid., Second ed. Oxford, Great Britain: Oxford University Press, 1959, pp. 510.

### Example 5

### Bioreactor Having 3D Microfabricated Capillary Networks

An innovative three dimensional microfabrication technology coupled with numerical simulation is implemented to enhance the mass transport in tissue culture. The core of this microfabrication technology is a high-resolution projection micro stereo lithography (μSL) using a spatial light modulator as the dynamic mask. This unique technology provides a parallel fabrication of highly complex 3D microstructures. In this work, a set of poly (ethylene glycol) bioreactors are demonstrated with PμSL technology. Supported by the results of our numerical study and preliminary yeast cell culture images, the precisely controlled capillary density (>150/mm²) in the polymer matrix and improved transport of nutrients through advection and diffusion represent the key advantages of our microfabricated bioreactors. These types of 3D microfabricated bioreactors are capable of providing in vitro cultured flaps for medical applications.

Reconstructive surgery is performed to recover functions and appearance of the damaged tissues, especially following major cancer resections and trauma. It is estimated that more than one million reconstructive surgery procedures are performed by plastic surgeons every year. And the reconstructive surgery has changed from “climbing ladder” to “riding elevator”^{[1]}, in which case flaps are preferably used in the reconstructive procedures. And the free flap is the most successful one. A free flap is a block of tissue with inherent microcirculatory network that usually is removed from another region of the patient that is relatively close to the defective site. It is based on the concept of angiosome^{[2]}. However the nature of sacrificing one part of body for another limits the application of free flap in practice. Alternative tissue sources for reconstructive surgery are desired.

Apart from the free flaps, culturing tissue in vitro using the patient's cells is the most attractive way to supply tissues for reconstructive surgery, since there is no foreign body reaction. However due to the lack of microcirculatory system at the earlier stage of the culture, no matter in vitro or in vivo, there is only very limited success. The time scale for revascularization is on the order of days (even with growth factors) and the time scale for cell death from hypoxia is on the order of hours. Therefore without capillary perfusion, the metabolism during cell growth cycle will eventually exhaust the supply of nutrient and oxygen from the external environment and the embedded cells suffer from the lack of nutrients, creating a bottleneck for the growth of thick (>1 mm scale) 3D tissues. Studies^{[3, 4]} confirm that the cells in the tissue are poorly cultured when they are further than ˜400 μm from the external nutrient source. As a matter of fact, in real tissue, almost all cells are within 100 μm from a capillary. Several studies have tried to enhance the mass transport in tissue culture with different approaches. For example, by inserting and extracting nylon strands and tubing, straight artificial blood vessels are cultured to deliver the culture medium.^{[5]}. However, no real vascular system is composed of straight capillaries and it is impossible to connect thousands of capillaries when transplanting in vitro tissue to the host body. Griffith, et al^{[6]} creatively introduce a three dimensional printing process to create three dimensional channels, but as the resolution of this technology is only about 200 μm, it cannot reliable produce capillary-sized vessel (e.g., having a vessel diameter less than 10 μm). Another study^{[7]} uses silicon microfabrication technology and molding to create two dimensional microchannels for enhanced mass transport. Nevertheless, the three dimensional nutrient transport in thick tissue culture still remains a big hurdle in tissue engineering. The current state of microvascular networks in tissue engineering is well addressed in various publications^{[8].}

A three-dimensional microfabrication technology, the Projection Micro-Stereolithography (PμSL)^{[9]}, is introduced and coupled with mass transport simulation for the design and fabrication of vascularized micro bioreactors. The micro fabricated bioreactor dramatically enhances the three dimensional mass transport by providing well controlled and tailored advection and diffusion through microfabricated capillaries. This microfabrication method brings several unique advantages to the advanced microbioreactor research and development: first, the capability of PμSL to build truly 3D sophisticated microstructures with very fine spatial resolution at micron scale; second, a significantly shortened design cycle enabled by high fabrication speed (1000 layers in a couple of hours)^{[9]}; finally, the choice of biocompatible and biodegradable polymers offers flexibility for fabricating implantable vascularised scaffold for different tissue culture^{[10, 11]} and/or applications.

Micro Fabrication and materials: The principle of projection micro-stereolithography is highlighted in FIG. 61 and further detailed elsewhere.^{[9]} The process starts by generating a 3D structure in Computer Aid Design (CAD) software, then slicing the structure into a plurality of layers that can are sequences of bitmap images according to the desired spatial resolution on the direction perpendicular to the slicing planes. Each image defines a polymer layer to be solidified in the later fabrication process. During one fabrication cycle, one image is read in and displayed through a spatial light modulator (SLM). The modulated light pattern is then delivered by the light path composed of beam splitter and 45° mirror to the reduction lens. The reduced image is focused on the photo curable liquid surface. The whole layer (usually 2-20 microns thick) is polymerized simultaneously. Optionally, the polymer resin is moved in an x-y plane by x-y controlled movement of the stage upon which the polymer is supported. After one layer is solidified, the polymerized part is immersed deep into the liquid surface to allow a new fresh thin liquid layer atop. A new fabrication cycle starts. By repeating the cycles, a 3D microstructure is formed from the stack of layers.

PμSL is compatible with various biomaterials of different functions, for example biocompatible and biodegradable polymers including Poly (ethylene glycol) (PEG), poly lactic acid (PLA), ploy caprolactone (PCL), and their copolymers. PEG is known as a biocompatible polymer. The monomer used in this example is a water-soluble

PEG diacrylate (molecular weight 575, from Sigma-Aldrich, with viscosity 57 cP at 25° C.). Bis(2,4,6-trimethylbenzoyl)-phenylphosphineoxide (Irgacure 819, from Ciba) is used as the photo initiator. Certain amount of UV absorber is mixed with the PEG monomer to control the UV penetration depth in the solution. Two representative 3D structures are shown in FIG. 62. In order to enhance the nutrient transport during the thick tissue culture, the micro fabrication technology must be able to make highly branching capillary tree structures as shown in FIGS. 62A and B; the inner radius of those capillaries vary from 10 μm to 30 μm. Similar to in vivo microcirculatory networks, the capillaries are fed by a larger diameter vessel (e.g., an arteriole) that decreased in diameter along the direction of flow. Capillary networks that branch from the feed arteriole can have an apex angle that is similar to those observed in the biological system. FIGS. 62C and D have a very different linear geometry compared to that in A and B. FIGS. 62C and D are different views of a 9 by 9 capillary array having 10 μm inner radius, 20 μm outer radius, 80 μm spacing and a length of 800 μm (aspect ratio >20, effective channel density >150/mm²). That system illustrates the excellent consistency of the resolution within the 3D fabrication domain. PμSL provides excellent capability and application in the fabrication of complex three dimensional microstructures having high aspect ratio (>40:1) and free standing structures. Table 14 shows the basic data of the PμSL system used in this example.

Not only is PμSL good at constructing high resolution 3D geometries, but it also has tremendous capability of locally controlling the mechanical and physical properties, such as young's modulus^{[12]} and permeability. The cross-linking ratio of photo curable polymer increases as the exposure dose increases and finally reaches a plateau^{[13]}. However the permeability of the photo cured polymer is a function of cross-linking ratio, the permeability decreases as the cross-linking ratio increases (FIG. 63). Therefore, controlling the illumination dose for each layer and within each layer in the fabrication flow, the permeability of the 3D structure can be precisely, locally tuned in a three dimensional space. Such localized control is useful in providing local delivery of material, such as release of a bioactive substance, in tissue engineering devices.

Vascularized MicroBioreactor: During tissue culture, the nutrients are delivered to the cells in the bioreactor via a series of capillary channels. It is very important that all the cells in the tissue receive adequate nutrient supply in order for the cells to reach high cell density. In normal tissue, almost no cell is farther than 100 μm from the nearest vessel, because the nutrients are depleted at that distance. It means for certain cell density, the capillary network has to be dense enough to provide a sufficient high level of nutrients to every cell to balance the consumption during cell metabolism. Similarly, in this microbioreactor design, we mimic the real nutrient delivery using capillary networks of high density. In these initial studies, mathematical complexity is reduced by providing a plurality of straight capillaries, thereby allowing easier examination and validation of the system. However, the systems and processes disclosed herein are particularly well suited to mimic geometrically complex branching capillary networks found in the body. In the present approximation, the real capillary network is modeled as an assembly of many segments of straight capillaries. Therefore we design and fabricate the micro bioreactor using PμSL as shown in FIG. 64. The capillaries are 800 μm long with 20 μm inner radius and 40 μm outer radius, the maximum distance between the nearest points of two adjacent parallel tubes is 40 μm. Two ring structures as “artery” (“inlet port”) and “vein” (“outlet port”) are connected to the bioreactor chamber, which are filled with parallel capillaries. The external nutrient supply is connected to the upstream ring, which has 400 μm inner diameter and feeds the capillary network. Fluid that has transited the capillary network exits the network at the downstream (e.g., vein) ring structure. FIG. 64D shows the cross-section view of the microbioreactor. Since the volume of the reactor is 0.16 μL, it allows culturing about 2,000 cells at the level of 10⁷ cells/cm³. Larger volume reactors may be made, such as about 1000 mL, thereby providing for culturing larger number of cells and/or providing larger-size implants.

Instead of experimental “trail and error”, numerical methods are used to study the mass transport by a PEG micro tube to assist in further bioreactor design and cell culture experiments. The details of the simulation of the micro bioreactor are reported elsewhere^{[14]}. Briefly, it is a diffusion limited problem with static governing equations:

Dₚᵢ∇²cᵢ=0 In the capillary wall (e.g., no consumption) (31)

Dₜᵢ∇²cᵢ=0 In cell suspension (e.g., cells consuming nutrient i) (32)

Here Dₚᵢ, Dₜᵢ are the diffusion coefficients for metabolite species i in polymer and in tissue respectively, they are assumed to be constant. cᵢ and Rᵢ are the concentration and consumption rate by the cultured cells of species i. In the case of steady state, the process of cells consuming metabolites is often described by Michaelis-Menten kinetics^{[15, 16]}:

$${R}_{i} = \frac{{V}_{max} {c}_{i}}{{K}_{M} + {c}_{i}} \qquad ( 33 )$$

Where Vₘₐₓ is the maximal uptake rate and K_{M} is the metabolite concentration when the uptake rate is half of the maximum. In Michaelis-Menten kinetics, the consumption behavior follows first order kinetics at low concentration. That means the consumption rate is proportional to the concentration. As the concentration of the metabolite increases, the consumption behavior will become zero order kinetics gradually. At certain point, the cell is saturated and the intake of metabolites reaches a plateau.

Yeast cell Saccharomyces cerevisiae is used as a model and the D-glucose transport in the bioreactor is calculated. S. cerevisiae cell growth has two phases^{[17]}: Glucose was first catabolized fermentatively into carbon dioxide and ethanol, and then when the glucose is limited (<830 nmol/cm³), ethanol was respired to carbon dioxide and water in the presence of oxygen. The biomass production rate in the second phase is much slower than in the first phase. Therefore, we simulate the first phase as the “lower bond scenario” to determine the biomass production in the microbioreactor. In this experiment we attempt to inhibit the second phase by removing the ethanol. The effective diffusion coefficient of glucose in crosslinked PEG (MW575) is measured using the method mentioned in^{[18]}, instead of studying permeability, we measured the effective diffusion coefficient. The flow rate in the channels is set at 0.5 mm/s. The Michaelis kinetic constants V_{MAX} and K_{M} are from^{[17]}. The average protein and biomass weight of single Saccharomyces cerevisiae yeast are 6×10⁻¹² g and 15×10⁻¹² g^{[19]}, respectively. From scanning electronic microscopy measurements, the diameter of the cultured S. cerevisiae yeast cells (strain INVSc1) is 3.14±0.61 μm (FIG. 65). When the cells are loosely packed so that a cell only occupies the eight vertices of a cube, the cell density is 3.2×10¹⁰/mL. The other simulation parameters are set as follows: Dₚᵢ=1.1×10⁻⁹ cm²/s, Dₜᵢ=1.1×10⁻⁶ cm²/s^{[20]}, C₀=110 μmol/cm³ (concentration of glucose in the polymer capillaries), V_{MAX}=663 nmol/mg protein/min, K_{M}=76 μmol/cm³.

The simulation indicates that the bottleneck of effective glucose transport is the permeability of the polymer materials. The glucose concentration drops off more than 95% after diffusing through the capillary wall. The simulation showed that if the center to center distance of the capillaries is set to 120 μm and the wall of the capillary is 10 μm, then the inner radius of the capillary has to be larger than 20 μm to ensure that all the yeast cells in the bioreactor has a high enough (>830 nmol/mL) glucose concentration to stay in the mixed repiro-fermentative metabolism and produce ethanol (FIG. 65). This configuration corresponds to 80.2 capillaries/mm² if the capillaries are in hexagonal arrangement. By increasing the inner radius of the capillary, not only the advection of the culture medium increased, but also the gap between capillaries is decreased (for fixed center-to-center separation distance). Increasing the lumen size of the capillary increases the capillary density (and of course correspondingly decreases the volume available to culture cells). When the inner radius is 20 μm, the lowest glucose concentration in the bioreactor is 880 nmol/cm³. When glucose concentration is lower than 830 nmol/cm³, S. cerevisiae yeast cell begin to consume ethanol^{[}17, 21] and the biomass growth becomes much slower than at higher glucose concentration. Two experiments at different points of the simulation curve are also showed in FIG. 65. Experiment A is in the Phase I region that the glucose concentration in the bioreactor is much higher than 830 nmol/cm³. Experiment B is at the cutoff region between Phase I and Phase II. We observe a dramatic difference of the biomass production between the two experiments. In experiment A, the yeast cells filled the bioreactor and even pushed the top cells out of the bioreactor during culture (FIG. 66A). When the bioreactor was removed from the culture chamber, the top layer of yeast actually was washed away. However, experiment B suggests the amount of yeast to fill the bioreactor was achieved (FIG. 66B). According to the simulation, in experiment B, the yeast cells should have buried all the capillaries in the bioreactor in a manner similar to experiment A. One of the possible reasons is that the Michaelis kinetic constants V_{MAX} and K_{M} applied in the simulation underestimate the yeast glucose consumption in our experiment. Another reason is that the simulation is based on the final steady state, assuming that the yeast have already filled the bioreactor and buried the capillaries. But in reality, the number of yeast increases from the few cells that are initially seeded. During yeast population growth increase, there is tension between the yeast number and the glucose concentration in the bioreactor. It is possible that there is a glucose concentration deficit at certain time (e.g., cell population/density) that must be overcome and the bioreactor system in experiment B was unable to overcome this deficit and reach the simulated steady state.

Yeast cell culture: Although the system and methods disclosed herein are compatible with any cell type, yeast cells are used because they are relatively simple and easy to culture compared to mammalian cells. This provides an easier system to test the advection and diffusion mass transport mechanism details. Yeast cell Saccharomyces cerevisiae is well studied, and so is used as a model to test the function of the micro fabricated bioreactor. The yeast is diploid strain INVSc1 (Invitrogen). Before yeast culture, the bioreactors are fabricated using PμSL and kept in 100% ethanol for 24 hours and biological-grade water for 24 hours to remove the residue irritant monomer and initiator, and also to increase the permeability of the capillaries. The yeast suspension in a 1.5 mL microcentrifuge tube is moved from −70° C. freezer and left at 20° C. room temperature for 20 minutes before seeding in the micro bioreactor using 0.1-10 μL micro pipette. The number of seeded yeast is about 80. The micro bioreactor is placed in the reaction chamber (1 inch×0.5 inch×0.5 inch) filled with DPBS (Dulbecco's Phosphate Buffered Saline). Two steel micro tubes with OD 400 μm penetrated the chamber side walls and are connected to the micro bioreactor inside as show in FIG. 64C. The chamber is covered with quarter inch thick transparent PLEX sheet to prevent possible contamination. The yeast culture medium YPD (1 g yeast extract (Difco), 2 g Peptone (Difco), 2 g D-glucose, 100 ml distilled water) is delivered to the capillaries in the micro bioreactor at an inlet flow rate of 0.5 mm/s. The culture chamber is kept in a humidified incubator at 30° C. for 45 hours. The DPBS solution in the chamber is replaced with fresh solution every 6-10 hours to remove the ethanol in the chamber. The glucose concentration in the replaced DPBS is measured using GlucCell™ glucose monitoring system. The incubated micro bioreactor is removed from the chamber and left in air and room temperature for one hour before sputter coating and SEM observation.

FIG. 67 shows the average glucose increasing rate in the DPBS solution in experiment A (FIG. 66). The glucose rate reflects the number of the yeast cell in the bioreactor. The more yeast cells the lower glucose rate. From FIG. 67, in experiment A, the yeast number in the bioreactor kept increasing during the culture. The glucose rate decreased almost 10 times, but the actual number of yeast increased more than 10000 times. We contribute this discrepancy to two reasons: First, in Michaelis-Menten kinetics, the glucose consumption rate of yeast varies with the local glucose concentration. The increase in yeast number in the bioreactor changes the glucose distribution and thus changes the overall relation of the yeast number and glucose consumption in a nonlinear fashion. The other reason is that not all the yeast cells are consuming glucose at the end of experiment. The yeast at the top are pushed too far away from the capillary where the local glucose concentration is too low for the yeast to perform glucose metabolism^{[17].}

Conclusions: Projection Micro-Stereolithography provides rapid design and manufacturing of advanced microbioreactors by offering a unique opportunity to culture tissue flaps in vitro. By integrating high density microcapillary channels within the microbioreactors, mass transport is enhanced by advection to balance increasing demand of oxygen and nutrient during cell population increase in the bioreactor. Simulation based on glucose diffusion models showed that the bottleneck of effective transport is the diffusivity of the polymer material of the capillary. There is a dramatic decrease in glucose concentration between the level in the lumen and the level outside the external face of the vessel wall. The S. cerevisiae yeast cell culture verified the simulation prediction. Not only is this model applicable for glucose, but it is also applicable for the transport of other metabolites for other cells, and may be used to model transport of substances from the cells to the vessel. Mammalian cell cultures generally do not have the second phase metabolism, unlike S. cerevisiae cells. In this case, the simulation modeling provides a good basis for predicting how far the nutrients transport into the cell layer. With the predicted transport distance, the density of the polymer capillary may be precisely controlled to ensure that all the cells in the microbioreactor are in healthy nutrient state.

### REFERENCES FOR EXAMPLE 5

[1] Dunn R, Watson S. Why climb a ladder when you can take the elevator? Plast Reconstr Surg 2001, 107:283
[2] Taylor G I, Palmer J H. The vascular territories (angiosomes) of the body: experimental study and clinical applications. Br J Plast Surg 1987; 40,113-141
[3] Sutherland, R. M. et al, Oxygenation and differentiation in multicellular spherioids of human colon carcinoma. Cancer Res. 46, 5320-5329, 1986.
[4] Martin, I. et al, Method for quantitative analysis of glycosaminoglycan distribution in cultured natural and engineered cartilage. Ann. Biomed. Eng. 27, 656-662, 1999.
[5] Neumann T, Nicholson B S, Sanders J E. Tissue engineering of perfused microvessels. Microvasc Res 2003; 66:59-67
[6] Griffith L G, Wu B, Cima M J, Powers M J, Chaignaud B, Vacanti J P. In virto organogenesis of liver tissue. Ann NY Acad Sci 1997; 831:382-97.
[7] Jeffrey T. Borenstein, et al, Microfabrication Technology for Vascularized Tissue Engineering, Biomedical Microdevices 2002, 4:3, 167-175
[8] Ruben Y. K, Henryk J. S, Kevin Sales, Peter Butler, Alexander M. S, The roles of tissue engineering and vascularisation in the development of micro-vascular networks: a review, Biomaterials 26 (2005), 1857-1875
[9] C. Sun, N. Fang, D. M. Wu, X. Zhang, Projection micro-stereolithography using digital micro-mirror dynamic mask, Sensors and Actuators A, 121 (2005), 113-120.
[10] Buddy D. Ratner, Stephanie J. Bryant, Biomaterials:Where We Have Been and Where We Are Going, Annu. Rev. Biomed. Eng. 2004. 6:41-75
[11] Qingpu Hou, Paul A. De Bank, Kevin M. Shakesheff, Injectable scaffolds for tissue regeneration, J. Mater. Chem., 2004, 14: 1915-1923
[12] E. Manias, J. Chen, N. Fang, X. Zhang, Polymeric Micromechanical Components with tunable Stiffness, Appl Phys Lett, Vol. 79, No 11, 10 Sep. 2001, 1700-1702
[13] N. Fang, C. Sun, X. Zhang, Diffusion-limited photopolymerization in scanning micro-stereolithography, Appl. Phys. A, 2004, 79:1839-1842
[14] C. G. Xia, C. Sun, D. M. Wu, X. Zhang and Nicholas Fang, 3D Microfabricated Bioreactors, NSTI-Nanotech 2006, Vol. 2, 140-143
[15] Leonor Michaelis, Maud Menten. Die Kinetik der Invertinwirkung, Biochem. Z., 1913, 49:333-369
[16] G. E. Briggs, J. B. S. Haldane, A note on the kinetics of enzyme action, Biochem. J., 1925, 19:339
[17] Karin Otterstedt et al, Switching the mode of metabolism in the yeast Saccharomyces cerevisiae, EMBO reports 2004, Vol. 5, No. 5:532-537
[18] C. K. Colton, et al, Permeability Studies with Celluloisc Membranes, J. Biomed. Mater. Res. 1971, vol 5, 459-488
[19] Fred Sherman and James Hicks, Getting started with yeast, Methods in Enzymology 1991, 194:3-21
[20] Antonio A. Vicente, marian Dluhy, Eugenio C. Ferreira, Manuel Mota, Jose A. Teixeira, Mass transfer properties of glucose and O₂in Saccharomyces cerevisiae flocs, Biochemical Engineering Journal 1998, 2:35-43
[21] Verduyn C, Zomerdijk T P L, Van Dijken J P, Scheffers W A, Continuous measurement of ethanol production by aerobic yeast suspension with and enzyme electrode, Appl Microbiol Biotechnol 1984 19:181-185

### Example 6

### Full 3D Micro Fabrication with Sacrificial Structure

This example provides a novel method for fabricating full three dimensional (3D) micro structures and moving parts. The method is based on one of the free form technologies, projection micro stereo lithography (PμSL). It fabricates 3D micro structures and sacrificial structures simultaneously, layer by layer, using the same material. It not only includes all the advantages of PμSL, but also pushes PμSL to full 3D micro fabrication by accessing geometries that cannot be made by conventional PμSL. A key aspect of this method is that the etching rate of a photo-crosslinked polymer (e.g., 1,6-hexanediol diacrylate) in etchant (e.g., sulfuric acid and hydrogen peroxide) varies with the degree of polymerization. The exponential relation of the etching rate and UV exposure dose of crosslinked polymer provides the selectivity of the etchant to the sacrificial structures.

Driven by the great economic potential, tremendous efforts have been continuously pouring into the rapid development of micro electro mechanical system (MEMS). Low cost, highly efficient and reliable micro devices enabled by MEMS technologies are remarkably changing the life of the world, such as sensors, actuators, micro display chips, inkjet nozzle arrays and so on. None of these successes could be possible without the strong support from micro-fabrication technologies. Most of the micro fabrication technologies are derived from the mainstream IC industry. These silicon-based micromachining technologies contribute significantly to the advancement of MEMS technology. However silicon technologies show limitation in the fabrication of micro devices with three dimensional complex geometries. Combining the strong demand and the difficulty to fabricate complex three dimensional micro devices in the field of silicon based MEMS, scientists and engineers are developing new approaches to enable the three dimensional micro fabrication for different materials and certain applications. For example, the LIGA process is designed to build high aspect ratio micro-structure by incorporating thick resist layers under masked X-ray or laser irradiation [1]. Another approach involves high density plasma etching also creates high aspect ratio micro/nano structure by removing masked material [2]. Both technologies provide limited capability for building microstructure on the vertical direction. However, they are still two and a half dimensional fabrication technologies. The three dimensional micro fabrication remained a challenge until the introduction of free forming fabrication technology. Free forming fabrication (FFF) is any fabrication technology that fabricates three dimensional complex structures by assembling small elements together and it usually starts from and is driven by computer aided design. Good examples of it are rapid prototyping, 3D printing, and direct writing for macro scale (>1 mm) fabrication. As for micro scale fabrication, three dimensional laser chemical vapor deposition (3D-LCVD) technology fabricates the microstructures by laser-induced chemical vapor deposition (LCVD) [3]. Electrochemical fabrication (EFAB) technology has been developed as an extension to the LIGA process in order to fabricate complex 3D metal micro structures [4]. The electro-chemically deposited metal layers are defined as electrode masks and a planarizing procedure controls the layer thickness. Nevertheless, both 3D-LCVD and EFAB are limited by the specified material selection and they are unable to build hang-over and moving micro structures.

Another recent free forming micro fabrication technology is microstereolithography. The basic principle is the same as stereolithography. It builds micro structures in a layer by layer manner by confining the illumination to defined areas in a photo sensitive resin bath. Depending on how each layer is built, microstereolithography can be divided into two types, vector by vector microstereolithography and projection (or integrated) microstereolithography. The vector by vector microstereolithography was first introduced by Takagi [5] and Ikuta [6]; it builds a polymer layer by tracing a focused light beam on the polymer resin surface. It is a slow serial process. To overcome the speed limitation and inspired by micro display technologies, scientists incorporated the parallel scheme into microstereolithography, called projection (or integrated) microstereolithography (PuSL). The basis of this technology rests on the use of spatial light modulator (SLM) as a dynamic mask; it can be either a LCD chip or DLP chip. Both were first introduced by Bertsch [7, 8]. PuSL can build most of the 3D micro structures, basically bottom to up connected structures. However, due to the nature of layer by layer fabrication scheme in polymer liquid, the difficulty of building long distance hang-over structures, “ceiling lamp” like structures or moving parts comes from the fact that a thin layer of hang-over structure or unconnected layer can flow away from desired position in PuSL process. In macro scale, additional supporting structures, usually supporting rods, are introduced to solve the problem. After fabrication, those supporting structure are manually removed because they are usually small and easy to remove. However, it is impractical to replicate the same procedure in micro scale. If the supporting structure is large enough for supporting, it will break the micro structure when removed, since it is connected with the micro structure. On the other hand, if the supporting structure is small it will not be strong enough for supporting. Furthermore, not all the supporting structure is accessible from outside, but instead may be blocked by surrounding microstructures making it difficult or impossible to remove the supporting structure without damaging the microstructure.

In order to enable the full 3D micro fabrication in PuSL, sacrificial structures become necessary. Although sacrificial layer in PuSL has been used [9], the use involved sacrificial layers made from a material that is different than the material from which the sacrificial layer was made. In that technique, the sample needs to be switched from one solution to another, the cleaning, alignment and focusing process in each switch will dramatically reduce the fabrication speed. This impairs the advantages of PuSL. Methods disclosed herein may use of same material for the micro structure and sacrificial structure. The micro structure and sacrificial structure are fabricated simultaneously, thus maintaining fabrication speed. The key principle for this technique is that the etching rate of polymer in etchant varies with the degree of polymerization (with support structures corresponding to sacrificial elements having a lower degree of polymerization than the microstructures.

METHOD AND MATERIALS: Our full 3D micro fabrication technology is based on traditional PuSL[10], highlighted in FIG. 61. The process starts from generating a 3D structure in Computer Aid Design (CAD) software, and then slices the structure into a sequence of mask images (digital mask). Each image represents a thin layer of the 3D structure respectively. During a fabrication cycle, one image is displayed on the reflective LCD panel. The image on LCD is then focused onto the photocurable liquid surface. The whole layer (usually 2-20 microns thick) is polymerized simultaneously. After one layer is solidified, the polymerized part is immersed into the resin to allow a new thin liquid layer atop. By repeating the cycles, a 3D microstructure is formed from the stack of layers. One of the advantages of a digital mask over a physical mask is that a digital mask can create gray scale exposure light fields, while the physical mask can only create black and white, binary exposure light fields. The light intensity distribution of the reflective beam from the LCD panel is closely proportional to the grayscale distribution of the digital mask. It is well known that the degree of irradiation polymerization is related to the incident light intensity. The gray scale of digital mask provides the opportunity to control the degree of polymerization locally, thereby providing a means for controlling the etching rate of polymer in etchant. The etching rate decreases as the degree of polymerization increases. The quantitative study on the etching kinetics is presented hereinbelow.

In PuSL process, the 3D microstructure is generated layer by layer. The current layer must be precisely laid on top of the last layer. When the partially finished part is transported in the monomer solution, the last layer should stay where it is designed to be. In most of the cases, this is fulfilled, but for certain kinds of structures, such as long distance horizontally hang-over structure, “ceiling lamp” structure or moving parts, this will not happen. The fluid flow created during the structure transportation will apply hydraulic force on the structure causing the last layer to undesirably bend or float away before laying down the current layer. This will cause the structure collapse. To overcome this problem, we introduce the concept of a sacrificial supporting structure. The sacrificial structure is made from the same material as the micro structure and is fabricated using a digital mask that imparts a lower grayscale (e.g., light intensity) to the region of the photopolymerizable prepolymer corresponding to the desired location of the sacrificial element. Thus, the resultant sacrificial element has lower degree of polymerization and higher etching rate in etchant compared to the microstructures exposed to a higher grayscale. The monomer used in this example is 1,6-hexanediol diacrylate (SR238, Sartomer) and Ciba Irgacure819 as initiator. The wave length is 436 nm and the light intensity for total white digital mask is 4.2 mW/cm². The etchant is composed of one volume of 96% sulfuric acid and one volume of 30% hydrogen peroxide. This etchant is commonly used to clean the residual photoresist on the silicon wafer. FIG. 68 schematically shows one process for making 3D micro structure with sacrificial elements 6820 (FIG. 68A) and the actual finished samples after exposure to etchant (FIG. 68A). The sacrificial structure and micro structure are fabricated simultaneously. Whatever location a sacrificial structure is needed, the corresponding digital mask is set to a grayscale value as desired that is less than the grayscale value to which the final end product pattern is exposed. The grayscale area will cause lower degree of polymerization for the sacrificial structure. After the whole 3D structure is finished by the PuSL process, it is placed in good solvent of the monomer solution, such as acetone, for 24 hours at room temperature to remove the residue monomer in the structure. At the same time, a portion of the sacrificial structure is dissolved. The dissolved part usually is close to the edges of the sacrificial structure, where the degree of polymerization is even lower than the center area. After removing the residue monomer, the sample is placed in the etchant for a length of time, depending on the size of the sacrificial structure. The temperature of the etchant is set to 70° C. and stirred on a magnetic hot plate. In this example, the polymer structure is lighter than the etchant, so the magnetic stir creates a vortex and drags the micro structure into the etchant for isotropic etching (see FIG. 68C for end result microstructures). FIG. 69A shows the microstructure and sacrificial elements after the acetone treatment, but before the etchant step. FIGS. 69B, C shows the micro fabricated full 3D micro structure after the etchant step that removes the sacrificial elements. This is a hair tree with hairs pointing in all directions. It is clearly seen that without sacrificial structure (FIG. 69D) the disconnected elements float away randomly and collapse. But a portion of the “pointing downwards” hairs close to the center remain. This is because the region close to the center has higher light intensity and the sacrificial structure forms. The diameter of the hair varies from 60 μm to 100 μm. It is the first time that this kind of “fully 3D” structure at this scale can be made in a couple hours or less. One side effect of this process is that the surface roughness may increase after etching because for each layer there is also a gradient of degree of polymerization along the thickness direction. This will result in uneven etching on surfaces. In addition, an etchant for crosslinked polymer usually is very strong so that it may be a problem when the sample has to stay on a particular as the strong acid may possibly attack the substrate. Accordingly, in an embodiment the substrate that supports the resultant 3D structure generated by PμSL is selected from a material that is chemically inert or resistant to acid attack by the etchant.

KINETICS OF ETCHING: Polymer etching is a process of breaking down the chemical bonds of the polymer chain. So the linear etching rate will be determined by the density of the chemical bonds, especially in the case of surface etching. And surface etching is necessary for the full 3D microfabrication technology described herein. Surface etching only etches the surfaces so that the overall geometry of the micro structure is maintained. Bulk etching, however, may break larger pieces into smaller pieces and destroy the microstructure. This example indicates that the photocrosslinked 1,6-hexanediol diacrylate in our etchant undergoes surface etching (data not showed). The density of the chemical bonds is proportional to the amount of polymerized monomer in one unit volume of starting monomer, which is the degree of polymerization. Two parameters are readily controllable in this technology, light intensity and irradiation time for each layer. The light intensity is controlled by the grayscale of the digital mask and the irradiation time is controlled by the time that digital mask is displayed on the LCD panel. An individual pixel on the digital mask may also be selected to vary over time, so that regions corresponding to sacrificial elements may be selected to have shorter exposure times, lower intensities, different exposure wavelength, or any of these effects in combination. Provided below is the development of a semi-empirical theory to describe the kinetics of the etching.

The chemical reaction in the fabrication process is a radical chain polymerization. Because the volume of the resin is much larger than the volume of micro structure, to a very close approximation, the starting monomer and initiator concentration are the same for each layer. The entire fabrication process is done at constant room temperature. Accordingly, the reaction temperature is assumed to remaining unchanged for each layer and the temperature dependent parameters are treated as constants in the analysis. The rate of polymerization in radical chain polymerization can be expressed as [11]:

$${R}_{p} = {k}_{p} [ M ] {( \frac{φ {I}_{0} ( 1 - {e}^{- ɛ [ A ] b} )}{{k}_{t}} )}^{1 / 2} \qquad ( 34 )$$

where kₚ and kₜ is the rate constant for polymer chain propagation and termination, respectively, [M] is the monomer concentration, φ is the quantum yield for initiation, I₀ the incident light intensity, [A] is the concentration of species which undergoes photo-excitation, ∈ is the molar absorptivity (extinction coefficient) of A at the particular frequency of radiation absorbed and b is the thickness of reaction system being irradiated. The rate of polymerization is also called the rate of monomer disappearance, and it is given with very good approximation [11] by:

$$- \frac{d [ M ]}{d t} = {R}_{p} \qquad ( 35 )$$

Combining equations (34) and (35 and integrating provides:

$$[ M ] = ( {[ M ]}_{t = 0} - {[ M ]}_{t = ∞} ) exp ( - {{k}_{p} ( \frac{ϕ {I}_{0} ( 1 - {e}^{- ɛ [ A ] b} )}{{k}_{t}} )}^{1 / 2} t ) + {[ M ]}_{t = ∞} \qquad ( 36 )$$

The surface etching rate of the polymer is assumed to vary linearly with the density of chemical bond (or polymerized monomer), so it can be described by:

$$\begin{matrix}{R}_{etching} = α ( {[ M ]}_{t = ∞} - [ M ] ) + β \\ = - α ( {[ M ]}_{t = 0} - {[ M ]}_{t = ∞} ) exp ( - {{k}_{p} ( \frac{φ {I}_{0} ( 1 - {e}^{- ɛ [ A ] b} )}{{k}_{t}} )}^{1 / 2} t ) + α ( {[ M ]}_{t = 0} - {[ M ]}_{t = ∞} ) + β \\ = {C}_{1} exp ( - {{k}_{p} ( \frac{φ {I}_{0} ( 1 - {e}^{- ɛ [ A ] b} )}{{k}_{t}} )}^{1 / 2} t ) + {C}_{2}\end{matrix} \qquad ( 37 )$$

Here, c₁=−α([M]ₜ₌₀−[M]_{t=∞}), C₂=α([M]ₜ₌₀−[M]_{t∞})+β and α,β are constants. C₂ is the etching rate at maximum degree of polymerization. It is measured using full polymerized disk with diameter 5 mm and thickness 1.76 mm. The disk is etched in the same etchant under the same conditions mentioned in the last section for 5 hours. The measured c₂=(16±0.7)um/hr is close to the acid etching rate of photoresist. From Eq. (37), the controllable parameter for the process is light intensity I₀ and radiation time t. Due to the efficiency of the optical system, especially the reflective LCD panel, even with gray scale zero there is still non-zero background light intensity. And from our measured data (not showed), the relation of the reflected light intensity of LCD and the grayscale are very close to a linear one. In FIG. 70, we experimentally verify the etching rate power laws of light intensity I₀ and irradiation time t in Equ. 37. The etching rate exponentially changes with the product of t and I₀^{1/2}.

In summary, a three dimensional sacrificial structure is introduced to fabricate full three dimensional micro structures and micro moving parts for micro assembly. The sacrificial structure shares the same material as the micro structure. This technology extends the capability of current projection micro stereolithorgraphy method without impairing its advantages. The core of this technology relies on the fact that the etching rate of polymerized monomer depends on the degree of polymerization. We have developed the semi empirical theory to explain the etching kinetics and experimentally verified the power laws in the theory. This technology is not limited to the polymer and etchant provided in this example. Any photocurable polymer capable of undergoing surface etching in etchant is a valid candidate.

### REFERENCES FOR EXAMPLE 6

[1] E. W. Becker, W. Ehrfeld, P. Hagmann, A. Maner, D. Munchmeyer, 1986, “Fabrication of microstructures with high aspect ratios and great structural heights by synchrotron radiation lithography, galvanoforming, and plastic moulding (LIGA process)”, Microelectron. Eng. vol. 4, p35.
[2] S A McAuley, H Ashraf, L Atabo, A Chambers, S Hall, J Hopkins and G Nicholls, 2001, “Silicon micromachining using a high-density plasma source”, J. Phys. D: Appl. Phys. vol. 34, p 2769-2774.
[3] K. Williams, J. Maxwell, K. Larsson, M. Bioman, 1999, “Freeform fabrication of functional microsolenoids, electromagnets and helical springs using high-pressure laser chemical vapor deposition” in Proceedings of the IEEE International MEMS 99 Conference, p 232.
[4] A Cohen, G Zhang, F Tseng, U Frodis, F Mansfeld, P Will, 1999, “EFAB: Rapid, low-cost desktop micromachining of high aspect ratio true 3-D MEMS”, Proceedings of the IEEE international MEMS 99 conference, p244
[5] T. Takagi and N. nakajima, 1993, the 4^{th} International Symposium on Micro Machine and Human Science (MHS'93)
[6] Ikuta and K. Hirowatari, 1993, “Real three dimensional micro fabrication using stereo lithography and metal molding”, the 6^{th} IEEE Workshop on Micro Electro Mechanical Systems (MESMS'93), p42
[7] A. Bertsch, S. Zissi, J. Y. jezequel, S. Corbel and J. C. Andre, 1995, the 4emes assieses europeenes du prototypage rapide, Paris, France,
[8] L. Beluze, A. Bertsch, and P. Renaud, 1999, “Microstereolithography: a new process to build complex 3D objects”, the SPIE symposium on Design, Test and Microfabrication of MEMS/MOEMS, v 3680, pt. 1-2, p 808.
[9] Cabrera M, Bertsch A, Chassaing J, von Jezequel J y and Andre J C 1998, “Microphotofabrication of very small objects: pushing the limits of stereolithography”, Mol. Cryst. Liq. Cryst., vol. 315, 223-34
[10] C. Sun, N. Fang, D. M. Wu, X. Zhang, 2005, “Projection micro-stereolithography using digital micro-mirror dynamic mask”, Sensors and Actuators A, vol. 121, 113-120.
[11] George G. Odian, principles of polymerization, 2^{nd} edition, John Wiley & Sons, Inc. NY, 1981,179-222

The ability to generate fully 3D microstructures with sacrificial structures provides access to numerous applications. For example, artificial polymeric microvascular networks can be generated. Such a network has numerous applications such as for generating vascularized tissues, implantable tissues and for use in calibrating various medical techniques to better obtain in vivo information related to tissue activity, status, cancer diagnosis and other medically-related information about potential disease states. In addition, the capability to generate any 3D microshape in a polymer permits development of various MEMS devices such as microfluidic flow sensors, actuators, optical conduits, or embedded logic circuits for information processing.

Three dimensional microcapillary systems provided herein provide functional capabilities including mass transport from one location to a large volume of space and also mass collection from a large volume to a single location. On a large length scale, convection drives mass transport; on a small length scale diffusion drives the mass transport. In addition, various forces can be used to drive mass including a pressure difference (convection-driving) or chemical gradient (diffusion-driving). The systems are useful in mimicking various biological tissues ranging from blood vessels to plant roots and leaves, for example. Other applications of microcapillary or microvascular systems include affinity oxygenators, heat exchangers, self-healing (e.g., wound healing, tissue defect healing) and tissue engineering.

Provided herein are various fabrication modes, including single, step and multiple fabrication modes. Representative examples of some different microstructures generated by processes and methods described herein are shown in FIG. 71. The capability of generating three-dimensional networks of microstructures of any geometry facilitates application of the process to a number of disciplines ranging from artificial microvasculature and tissue engineering (FIG. 71A), bioreactors for generating a biological material (FIG. 71B), microfluidic devices and MEMS (FIG. 71D) and other microstructures having an arbitrary shape or configuration with respect to surrounding structures such as overhang structures and movable parts that may be physically inaccessible after the desired shape is desired.

In general, most current microfabrication technologies provide two-dimensional structure generation, or at most limited three-dimensional structure generation (for PμSL), as summarized in FIG. 72. It is particularly difficult to generate by PμSL structures having a geometry illustrated in FIG. 72B, where the angle with an underlying connecting element is less than 90°. In contrast, structures where the angle is greater than 90° (FIG. 72A) are generally accurately and reliably generated. The functional result of such geometric limitation is shown in FIG. 72C, where structures connected to the central element with angles less than 90° are bent and sagging. Using sacrificial support elements permit those structures to be accurately and reliably generated, as illustrated in FIG. 72D. Such full three-dimensional structure generate permits manufacture of cantilever and “ceiling lamp” type of structures not possible in conventional PμSL processes.

One example of a ceiling lamp or overhang element 6830 is provided in FIG. 68 (top left panels), where an element is suspended from sides and physically separated from the underlying floor (such that there is no underlying support). Similarly, an element capable of movement without breaking, or movable element 6840, is shown in FIG. 68 (top right panels, showing a movable ring element). As illustrated, the microstructure 6810 and the sacrificial structure 6820 are made of the same polymer material and may be fabricated simultaneously in a single step of illumination of a photopolymerizable polymer. This process is significantly faster compared to conventional methods for making MEMS and does not require lengthy and technical intermediate processing steps. Based on different degree of polymerization between the sacrificial element 6820 and the microstructure element 6810, the difference in etching rate during exposure to a polymer etchant provides selective removal of sacrificial element 6820, leaving behind microstructural elements 6810 that form a polymerized pattern layer, with multiple layers making up the end three-dimensional network geometry.

FIG. 73 summarizes the advantages of a digital mask compared to a physical mask for generating a controlled illumination distribution over an underlying surface (e.g., corresponding to the surface of a photocurable prepolymer). A digital mask using, for example, an LCD, is capable of spatially and/or temporally controlling light intensity provided to a to-be-polymerized prepolymer. As used herein, “gray scale” is exemplified in FIG. 73A (left panel), where there is a gradation of light intensity values. In contrast, a conventional physical mask provides only a binary light intensity field (e.g., on/off), as summarized in FIG. 73B. The etching rate of etchant on a polymer is related to the degree of polymerization, which is in turn affected by light intensity. Accordingly, the digital mask provides a means for selectively controlling etch rate distribution over a polymer region layer. For example, three or more polymerization states may be generated: a first region that is unpolymerized (where no or only background light is imparted to the photocurable prepolymer and is insufficient exposure to generate a polymer); a second region corresponding to a structural element that is polymerized with a polymerization density that provides a structural etch rate; a third region corresponding to a sacrificial element that is polymerized with a polymerization density that provides a sacrificial etch rate, where the sacrificial etch rate is greater than the structural etch rate (see FIG. 73C). The difference in etch rates is produced, for example, by illuminating the second and third regions at different intensities, durations, or both.

A schematic illustration of polymerization and etching kinetics is provided in FIG. 74. A photocurable prepolymer 7410 (e.g., free monomers) is illuminated, such as by illumination with UV light 7420. In this example, one region is exposed to higher light intensity (Iᵢ), a longer duration (Δt₁), or both compared to a second region having a lower light intensity (I₁), a shorter duration (Δt₁), or both. Accordingly, the one region corresponds to a region that will contain a microstructure after final processing and the second region to a sacrificial element that may be later removed by a removal process, such as an etchant. For both illumination regimes, polymerization occurs, but at a different polymerization density. Free monomer can be removed with solvent treatment leaving behind a polymerized structure having two different regions with different cross-linking density. The lower cross-linked region, corresponding to a sacrificial element, is removed by introducing an etchant to the system.

As described hereinabove, etching kinetics can be analyzed mathematically by integration of the differential equation that describes the rate of photopolymerization and assuming the etching rate of the polymer varies linearly with the density of polymerized monomer. Such an analysis indicates that etching rate is exponentially related to the product of time and the square root of light intensity (e.g., see Eq. 37 and FIG. 70). Accordingly, in an aspect provided herein are methods to select etching rates in two regions that are different by selecting illumination intensity, duration of illumination or both. FIG. 69 summarizes two such regions (see FIG. 69A), with subsequent removal of the sacrificial region having a faster etch rate than the microstructure region (see FIGS. 69B-C) and the benefit of using such a sacrificial region that is a support structure (see FIG. 69D).

The processes provided herein permit manufacture of three-dimensional microcapillary systems for tissue engineering that are capable of sustaining a substantial biomass at physiologically-relevant density (e.g., about 10⁸ cells/cm³ or greater) by providing high density of vascularization (e.g., about 100 vessels/mm²). Micronetworks made by conventional techniques suffer from various drawbacks including the techniques used to make the networks are unable to fabricate arbitrary capillary systems of any geometry or the resolution is too low (e.g., greater than 200 μm). In addition, proper formation of capillaries is not controlled with the interconnection between vessels being quite poor. Finally, small size capillaries generally do not survive, and the larger capillaries are easily blocked. Provided herein are various vascularized bioreactors that overcome these problems (see, e.g., FIGS. 4 and 75).

FIG. 75 illustrates a vascularized bioreactor having an inlet port 7510 for introducing culture media (corresponding to arterial inflow), an outlet port 7520 for removing culture media (corresponding to venule outflow), and a three-dimensional network of microvessels 7530 having a wall 7540. The bottom panel of FIG. 75 schematically illustrates a single microvessel of the network of microvessels shown in the top panel. In particular, wall 7540 is permeable so that culture media containing various material required by cells 7550 fed by the vessel (e.g., O₂, C₆H₁₂O₆, or any other desired material) can pass from the vessel lumen 7570 to the surrounding volume 7560 in which the cells are suspended. Similarly, wall 7540 can also be made permeable to materials generated by cells 7550, including CO₂ or materials of interest for collection downstream from the outlet port (e.g., ethanol, a drug, a prodrug, a material a cell has been genetically manipulated to overproduce). The permeability of wall 7540 to materials in both directions is illustrated by the two directions of arrows across the vessel wall. Such vascularized bioreactors can have easily controlled and monitored micro environments, such as bathing the cells in a biologically-compatible media (e.g., PBS), temperature, CO₂, pH, etc.

Various yeast cell culture devices are provided that are useful for generating various materials, including but not limited to ethanol (see, e.g., FIGS. 64-67). Similarly, the networks can support other cell types, including but not limited to CHO cells (FIG. 76), fibroblasts (FIG. 78) and cancer cell models. The capability of supporting cancer cells in a realistic microvascular network facilitates calibration of medical imaging instruments used to detect cancerous cells or tumors in vivo such as by ultrasound or MRI, for example.

Also provided are methods of controlling the porosity of the vessel wall, including the size and density of the pores. For example, pore size can be varied from a diameter ranging from 20 nm to 900 nm, along with pore density (see FIG. 77). In an embodiment, pore size is controlled by mixing PEG with HDDA with different volume fractions. When the mixed solution is patterned, such as by PμSL, HDDA is fully cross-linked while PEG remains dissolvable. In this manner, porous microbioreactors are made which can be designed to be more permeable to proteins and other biologic materials having larger molecular weight. Pore size is optionally varied along the network to allow so-called functionally graded density and permeability structures. In an embodiment, this is achieved by tuning light intensity and exposure time at each layer, while maintaining PEG/HDDA composition.

FIG. 79 illustrates use of a network disclosed herein for tissue engineering. A plurality of cell populations are introduced to the microcapillary system, such as endothelial cells, smooth muscle cells, and cells corresponding to the tissue of interest, as desired (FIG. 79A). Appropriate culture media is introduced to the lumen of the microvessel having a permeable wall. The porous nature of the vessel wall (FIG. 79B) scaffold increases mass transport. Selecting a polymer that is biodegradable facilitates break-down of the polymer so that a “natural” blood vessel replaces the artificial microvessel.

One advantage of the present system and process is that any microvascular geometry can be created efficiently, accurately and reliably, such as the microvascular network depicted in FIG. 79C. Various tissue engineering aspects may be addressed, such as developing cancer models (e.g., blood vessel networks and cancerous tumors) including a fibroblase and epithelial cell culture that modes prostate and prostate cancer and for tissue regeneration including implanting a device into a bone tissue defect to promote bone regeneration. Finally, the present processes and devices make tissue models on demand possible. Such tissue models have numerous applications beyond tissue engineering and growth. For example, in the medical imaging application area the cultured tissues and microvessels provided herein can be used as “phantoms” to calibrate the resolution of functional imaging instruments such as MRI or ultrasound. One advantage of the models provided herein is that they provide a contrast close to real in vivo tissues found in clinical settings, but are more readily controlled, so that the actual disease (e.g., trauma, infection, cancer, genetic-based disorders) can be better modeled and mimicked. For example, the defect in microvessels, tumor size, or tumor type can be precisely modeled, thereby improving diagnosis, detection and resolution, for example.

Every formulation or combination of components described or exemplified herein can be used to practice the invention, unless otherwise stated.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

Whenever a range is given in the specification, for example, a temperature range, a size range, a percent range, a time range, or a composition or concentration range, all intermediate ranges and subranges, as well as all individual values included in the ranges given (e.g., within a range and at the ends of a range) are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, materials, reagents, synthetic methods, purification methods, analytical methods, assay methods, and methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

TABLE 1
Comparison of Popular Micromachining Technologies

| | | Res | | |
|---|---|---|---|---|
| Tech | Materials | (μm) | Throughput | Features |
| Micro- | Polymers | 1-5 | High with | 3D |
| stereolithography | | | projection | |
| 3D Printing | Powdered Metals, | 50 | low | 3D |
| | Polymers, & | | | |
| | Ceramics | | | |
| Selective Laser | Powdered Metals, | 400 | low | 3D |
| Sintering | Polymers, & | | | |
| | Ceramics | | | |
| Fused Deposition | Polymers, Wax | 250 | low | 3D |
| Modeling | | | | |
| Soft Lithography | Most | 0.5 | high | 2D |
| Laser Machining | Metals, Plastics | 5-10 | med | Limited |
| | | | | 3D |
| Micromachining | Most | 1 | med | 2D |

TABLE 2
Electrode Layer Summary

| | | | | Dielectric | |
|---|---|---|---|---|---|
| Layer | Material | Thickness | | Const. [−] | Process |
| 4 & 5 | Teflon AF 1600 - | 2 layers, each | | 1.93 | Dipcoat & |
| | DuPont | ~1.75 | um | | Cure |
| 3 | Polyimide 5878G | ~7 | um | 3.3 | Spincoat & |
| | | | | | Cure |
| 2 | Nickel Electrode | ~200 | nm | NA | E-beam |
| | Layer | | | | Evaporation |
| 1 | Chromium Adhesion | ~20 | nm | NA | E-beam |
| | Layer | | | | Evaporation |

| | | | | |
|---|---|---|---|---|
| 0 | Silicon Substrate | NA | NA | NA |

TABLE 3
Resistivity of Salted and Unsalted Monomer Solutions

| | Resistance |
|---|---|
| Monomer Solution | [kΩ] |
| PEGDA-258 | 70,000 |
| PEGDA-258 + NaCl | 94,000 |
| PEGDA-258 + ITFMS 0.3% | 600 |
| PEGDA-258 + ITFMS 1.0% | 130 |
| PEGDA-258 + ITFMS 5.0% | 22 |
| Tap Water (Champaign-Urbana, IL) | 16 |

TABLE 4
System Resin Contact Angles on Spuncoat Teflon

| Fluid | Ave. CA | StDev | StDev % of Ave |
|---|---|---|---|
| DI H2O | 122.0 | 1.14 | 0.9% |
| Tap H2O | 121.8 | 0.88 | 0.7% |
| SPEGDA-258 | 87.2 | 1.59 | 1.8% |
| SPEGDA-258A | 85.2 | 2.07 | 2.4% |
| SPEGDA-575 | 92.7 | 2.92 | 3.1% |
| SPEGDA-6000 | 105.1 | 2.80 | 2.7% |
| Octane | 47.5 | 1.39 | 2.9% |

TABLE 5
System Resin Contact Angles on Dipcoated Teflon

| Fluid | Ave. CA | StDev | StDev % of Ave |
|---|---|---|---|
| DI H2O | 119.6 | 1.4 | 1.2% |
| Tap H2O | 117.6 | 1.1 | 1.0% |
| SPEGDA-258 | 89.1 | 1.9 | 2.2% |
| SPEGDA-258A | 86.4 | 1.5 | 1.7% |
| SPEGDA-575 | 99.3 | 1.8 | 1.8% |
| SPEGDA-6000 | 102.4 | 1.0 | 1.0% |
| Octane | 48.3 | 0.7 | 1.4% |

TABLE 6
Measured Surface Energies of System Resins

| Fluid | γ [mJ/m²] | ρ [g/cm³] |
|---|---|---|
| DI H2O | 69.7 | 1.0 |
| Tap H2O | 71.0 | 1.0 |
| SPEGDA-258 | 37.0 | 1.1 |
| SPEGDA-258A | 37.2 | 1.1 |
| SPEGDA-575 | 38.0 | 1.1 |
| SPEGDA-6000 | 55.5 | 1.2 |
| Octane | 21.4 | 0.7 |

TABLE 7
Published Surface Energies of Sartomer Resins [39]

| Code | Resin | γL [mJ/m²] |
|---|---|---|
| SR210 | PEGDA-200 | 37.2 |
| SR603 | PEGDA-400 | 40.0 |
| SR610 | PEGDA-600 | 41.6 |

TABLE 8
Interfacial Energies for System Fluids on Teflon 1600 AF Surface

| | γ_{T-Fluid} |
|---|---|
| System Resin | [mJ/m²] |
| Di Water | 50.1 |
| Tap Water | 48.6 |
| Octane | 1.4 |
| SPEGDA-258 | 15.1 |
| SPEGDA-258A | 13.3 |
| SPEGDA-575 | 21.9 |
| SPEGDA-6000 | 27.6 |

TABLE 9
“Flat” Voltages for Monomers on Teflon 1600 AF Surface

| Fluid System - Flat Cond | Voltage [V] |
|---|---|
| SPEGDA-258/Octane | 97 |
| SPEGDA-258A/Octane | 90 |
| SPEGDA-575/Octane | 118 |
| SPEGDA-6000/Octane | 134 |

TABLE 10
Dₚ for PEG Solutions at 435 nm

| Fluid | Absorber % | Trans Ratio | Dp |
|---|---|---|---|
| PEG 258 | 0.50 | 0.68 | 39 |
| PEG 258 | 0.25 | 0.78 | 62 |
| PEG 258 | 0.10 | 0.87 | 110 |
| PEG 258 | 0.05 | 0.93 | 213 |
| PEG 575 | 0.50 | 0.65 | 36 |

TABLE 11
First Five Roots of Equation 3.14 for Various Values of k [44]

| K | α₁ | α₂ | α₃ | α₄ | α₅ |
|---|---|---|---|---|---|
| 1.5 | 6.2702 | 12.5598 | 18.8451 | 25.1294 | 31.4133 |
| 2 | 3.1230 | 6.2734 | 9.4182 | 12.5614 | 15.7040 |
| 2.5 | 2.0732 | 4.1773 | 6.2754 | 8.3717 | 10.4672 |
| 3 | 1.5485 | 3.1291 | 4.7038 | 6.2767 | 7.8487 |
| 4 | 1.0244 | 2.0809 | 3.1322 | 4.1816 | 5.2301 |

TABLE 12
Diffusion Coefficients along the Eight Matrix Axes at t = 0 s

| Axis | D [μm²/s] |
|---|---|
| 1 | 73 |
| 2 | 79 |
| 3 | 51 |
| 4 | 45 |
| 5 | 45 |
| 6 | 45 |
| 7 | 51 |
| 8 | 56 |

TABLE 13
Diffusion Coefficients for the Eight Matrix Axes
of the 2D Diffusion Pinwheel at Two Time Steps

| | t = 5 s | t = 10 s | STDev |
|---|---|---|---|
| Axis | D [μm²/s] | D [μm²/s] | [%] |
| 1 | 73 | 68 | 6% |
| 2 | 79 | 73 | 5% |
| 3 | 51 | 56 | 7% |
| 4 | 45 | 45 | 0% |
| 5 | 45 | 45 | 0% |
| 6 | 45 | 56 | 16% |
| 7 | 51 | 51 | 0% |
| 8 | 56 | 73 | 18% |

TABLE 14
Basic data of Projection Micro Stereolithography
system Of Example 5

| Resolution | Speed | Sample Size |
|---|---|---|
| 2 μm (in plane) | 4 mm³/hour | 1.5 mm × 1.1 mm × |
| 1 μm (off plane) | (viscosity 10 cP) | 3.5 mm |

We claim:

1. A method of making a microvascularized bioreactor, said method comprising:
a. providing a photocurable liquid composition having a top surface;
b. providing a source of light capable of curing at least a portion of said composition;
c. illuminating said composition top surface with said light, wherein said illumination is in a pattern thereby simultaneously generating a polymerized pattern layer having a layer thickness;
d. immersing said polymerized pattern layer into said composition depth by a vertical displacement corresponding to said layer thickness;
e. waiting a surface dwell time for said surface to become substantially level;
f. repeating said illuminating step to generate an adjacent polymerized pattern layer, wherein said repeating step is repeated for a number of steps to generate a microvascular network having an interior surface and an exterior surface; and
g. contacting said exterior surface with a cell population, thereby obtaining said microvascularized bioreactor;
wherein said microvascular network has a permeability to a biological material that varies with location within said network.

2. The method of claim 1 wherein said permeability is selected to optimize diffusion of said biological material through a network wall between said interior and exterior surfaces.

3. (canceled)

4. The method of claim 2, wherein said permeability varies along a longitudinal direction or a radial direction.

5-7. (canceled)

8. The method of claim 1 further comprising:
providing gray scale illumination in at least one illumination step,
wherein said gray scale illumination is provided by a gray scale mask having a plurality of pixels, each pixel capable of providing a plurality of grayscale shades each having a unique intensity.

9. (canceled)

10. The method of claim 8, wherein said gray scale illumination has a minimum illumination intensity sufficient to cause polymerization, so that said gray scale illumination generates a polymerized layer with variable cross-linking, thereby providing variable permeability in said polymerized layer.

11. The method of claim 8, wherein said gray scale illumination has a minimum intensity insufficient to generate polymerization, thereby providing a polymerized layer having features with different heights.

12-16. (canceled)

17. The method of claim 1, further comprising an electrowetting step to reduce surface dwell time, said electrowetting step comprising:
a. providing a two-fluid interface operably connected to said top surface, wherein one fluid is conductive and the other fluid is non-conductive; and
b. applying a voltage to said conductive fluid to flatten said interface, thereby decreasing said surface dwell time.

18. The method of claim 17 wherein said two-fluid interface comprise said one fluid that is polyethylene glycol diacrylate (SPEGDA) conductive fluid on the bottom said other fluid is octane nonconductive fluid on top.

19. (canceled)

20. The method of claim 1, further comprising:
a. obtaining an in silico image of an in vivo microvascular network, wherein said image comprises a plurality of layers; and
b. using each of said layers to generate each of said polymerized layers thereby making a microvascular network having a geometry corresponding to said in vivo microvascular network.

21-24. (canceled)

25. The method of claim 1, wherein said microvascular network has an upstream inlet port and a downstream outlet port, said method further comprising:
a. introducing a culture media capable of sustaining said cell population to said inlet port at an inlet flow-rate; and
b. removing said culture media that has transited said microvascular network at said outlet port
wherein said cell population ation produces a compound that diffusues from said exterior surface to said interior surface, and is subsequently collected at said outlet port.

26-35. (canceled)

36. The method of claim 1, wherein said illuminating step comprises
a. illuminating a first region with a first light exposure; and
b. illuminating a second region with a second light exposure, wherein said second light exposure has an intensity that is less than said first light exposure intensity, or said first second light exposure has a duration that is less than said first light exposure duration, or both;
thereby generating a polymer in said first region that has a cross-linking density that is greater than said second region cross-linking density.

37. The method of claim 36 further comprising selecting said first and second light exposure intensity, duration, or both to generate an etch rate for said first region polymer and said second region polymer when exposed to an etchant that is at least 10 times different from each other.

38. (canceled)

39. The method of claim 36, wherein said first region polymer is a microstructure that is a part of said microvascular network and said second region polymer is a sacrificial element, said method further comprising the step of contacting said sacrificial element with an etchant to at least partially remove said sacrificial element.

40-42. (canceled)

43. A method of making a three-dimensional device, said method comprising:
a. providing photocurable liquid composition having a top surface;
b. providing a light source capable of curing at least a portion of said composition;
c. illuminating said composition top surface with gray scale illumination, wherein said gray scale illumination is a pattern of light intensity or duration that generates a pattern of polymer having a spatially varying cross-linking density; and
d. contacting said polymer with an etchant that selectively removes polymer having a lower cross-linking density;
thereby making a three-dimensional device.

44. The method of claim 43 further comprising making a plurality of polymer layers by:
a. illuminating said composition top surface with said light source, wherein said illumination is in a pattern thereby simultaneously generating a polymerized pattern layer having a layer thickness;
b. immersing said polymerized pattern layer into said composition depth by a vertical displacement corresponding to said layer thickness;
c. waiting a surface dwell time for said surface to become substantially level; and
d. repeating said illuminating step to generate an adjacent polymerized pattern layer, wherein said repeating step is repeated for a number of steps to generate a three-dimensional structure having at least one element that is a sacrificial element having said lower cross-linking density that supports at least a portion of said three-dimensional structure during processing.

45. The method of claim 43, further comprising selecting said gray scale illumination to generate a first region of polymer that is a sacrificial element and a second region of polymer that is a microstructure, wherein said sacrificial element provides physical support to said microstructure, wherein said sacrificial element has an etch rate that is selected from a range that is at least 5 to 10 times greater than said microstructure etch rate.

46. (canceled)

47. The method of claim 45 wherein said microstructure comprises an overhang structure or a movable element.

48. (canceled)

49. A method of producing a biological material, said method comprising:
a. providing a vascularized bioreactor having a three-dimensional network of microvessels capable of fostering a cell population, wherein said network has
i. a polymeric wall with a lumen-facing side and a cell-facing side, wherein at least a portion of said wall is permeable to said biological material and said wall has a permeability that varies with microvessel position;
ii. an inlet port upstream of said network for introducing culture media to said network;
iii. an outlet port downstream of said network for removing said culture media;
b. obtaining an isolated cell population capable of producing said biological material or a precursor thereof;
c. contacting said cell population to at least a portion of said outward-facing side of said network wall;
d. culturing said cell population in said bioreactor by introducing a culture media to said inlet port to expose said network wall inward-facing side to said culture media; and
e. collecting said culture media that has transited said microvessel network at said outlet port;
wherein said cell population produces a biological material capable of diffusing from said cell population to said culture media via said network wall so that said collecting step collects at least a portion of said produced biological material, said biological material is a biofuel, pharmaceutical, drug, a prodrug, or any precursors thereof.

50-56. (canceled)

57. A vascularized bioreactor comprising:
a. a three-dimensional network of microvessels having a wall made of a biocompatible polymer, wherein said wall has at least one parameter that varies with a longitudinal or a radial position within the network;
b. an inlet port for introducing culture media to said network; and
c. an exit port where culture media is removed from said network.

58-67. (canceled)

68. A method of calibrating a medical device comprising:
a. providing a microvascular network made by the method of claim 1 that supports a cell population;
b. introducing a challenge to said network;
c. imaging said microvascular network with a medical instrument to generate output data; and
d. calibrating said medical instrument with said output data.

69-71. (canceled)
